SEQUENCE LISTING (245 sequences)

SEQ ID NO: 1 - RNA, 21 nt, Mus musculus
ugagguaguagguuguauagu

SEQ ID NO: 2 - RNA, 22 nt, Mus musculus
ugagguaguagguugugugguu

SEQ ID NO: 3 - RNA, 22 nt, Mus musculus
ugagguaguagguuguaugguu

SEQ ID NO: 4 - RNA, 21 nt, Mus musculus
agagguaguagguugcauagu

SEQ ID NO: 5 - RNA, 22 nt, Mus musculus
cuauacgaccugcugccuuucu

SEQ ID NO: 6 - RNA, 21 nt, Mus musculus
ugagguaggagguuguauagu

SEQ ID NO: 7 - RNA, 21 nt, Mus musculus
ugagguaguagauuguauagu

SEQ ID NO: 8 - RNA, 21 nt, Mus musculus
ugagguaguaguuuguacagu

SEQ ID NO: 9 - RNA, 21 nt, Mus musculus
ugagguaguaguuugugcugu

SEQ ID NO: 10 - RNA, 21 nt, Mus musculus
uggaauguaaagaaguaugua

SEQ ID NO: 11 - RNA, 22 nt, Mus musculus
aacccguagauccgaacuugug

SEQ ID NO: 12 - RNA, 22 nt, Mus musculus
uacaguacugugauaacugaag

SEQ ID NO: 13 - RNA, 22 nt, Mus musculus
uacaguacugugauagcugaag

SEQ ID NO: 14 - RNA, 23 nt, Mus musculus
agcagcauuguacagggcuauga

SEQ ID NO: 15 - RNA, 22 nt, Mus musculus
caaagugcuaacagugcaggua

SEQ ID NO: 16 - RNA, 21 nt, Mus musculus
uaaagugcugacagugcagau

SEQ ID NO: 17 - RNA, 23 nt, Mus musculus
agcagcauuguacagggcuauca

SEQ ID NO: 18 - RNA, 23 nt, Mus musculus
uacccuguagauccgaauuugug

SEQ ID NO: 19 - RNA, 22 nt, Mus musculus
cccuguagaaccgaauuugugu

SEQ ID NO: 20 - RNA, 23 nt, Mus musculus
ucccugagacccuuuaaccugug

SEQ ID NO: 21 - RNA, 22 nt, Mus musculus
ucccugagacccuaacuuguga

SEQ ID NO: 22 - RNA, 21 nt, Mus musculus
ucguaccgugaguaauaaugc

SEQ ID NO: 23 - RNA, 21 nt, Mus musculus
cauuauuacuuuugguacgcg

SEQ ID NO: 24 - RNA, 21 nt, Mus musculus
ucggauccgucugagcuuggc

SEQ ID NO: 25 - RNA, 22 nt, Mus musculus
ucacagugaaccggucucuuuu

SEQ ID NO: 26 - RNA, 22 nt, Mus musculus
ucacagugaaccggucucuuuc

SEQ ID NO: 27 - RNA, 22 nt, Mus musculus
cagugcaauguuaaaagggcau

SEQ ID NO: 28 - RNA, 22 nt, Mus musculus
uaacagucuacagccauggucg

SEQ ID NO: 29 - RNA, 22 nt, Mus musculus
uugguccccuucaaccagcugu

SEQ ID NO: 30 - RNA, 21 nt, Mus musculus
uugguccccuucaaccagcua

SEQ ID NO: 31 - RNA, 21 nt, Mus musculus
uaccacaggguagaaccacgg

SEQ ID NO: 32 - RNA, 22 nt, Mus musculus
uaacacugucugguaaagaugg

SEQ ID NO: 33 - RNA, 20 nt, Mus musculus
cauaaaguagaaagcacuac

SEQ ID NO: 34 - RNA, 22 nt, Mus musculus
ugagaugaagcacuguagcuca

SEQ ID NO: 35 - RNA, 24 nt, Mus musculus
guccaguuuucccaggaaucccuu

SEQ ID NO: 36 - RNA, 22 nt, Mus musculus
ugagaacugaauuccauggguu

SEQ ID NO: 37 - RNA, 22 nt, Mus musculus
ugagaacugaauuccauaggcu

SEQ ID NO: 38 - RNA, 22 nt, Mus musculus
ucagugcacuacagaacuuugu

SEQ ID NO: 39 - RNA, 22 nt, Mus musculus
ucagugcaucacagaacuuugu

SEQ ID NO: 40 - RNA, 22 nt, Mus musculus
ucuggcuccgugucuucacucc

SEQ ID NO: 41 - RNA, 22 nt, Mus musculus
ucucccaacccuuguaccagug

SEQ ID NO: 42 - RNA, 21 nt, Mus musculus
cuagacugaggcuccuugagg

SEQ ID NO: 43 - RNA, 22 nt, Mus musculus
ucagugcaugacagaacuuggg

SEQ ID NO: 44 - RNA, 22 nt, Mus musculus
uuaaugcuaauugugauagggg

SEQ ID NO: 45 - RNA, 22 nt, Mus musculus
uagcagcacauaaugguuugug

SEQ ID NO: 46 - RNA, 22 nt, Mus musculus
uagcagcacaucaugguuuaca

SEQ ID NO: 47 - RNA, 22 nt, Mus musculus
uagcagcacguaaauauuggcg

SEQ ID NO: 48 - RNA, 24 nt, Mus musculus
caaagugcuuacagugcagguagu

SEQ ID NO: 49 - RNA, 23 nt, Mus musculus
aacauucaacgcugucggugagu

SEQ ID NO: 50 - RNA, 22 nt, Mus musculus
aacauucauugcugucgguggg

SEQ ID NO: 51 - RNA, 22 nt, Mus musculus
aacauucaaccugucggugagu

SEQ ID NO: 52 - RNA, 22 nt, Mus musculus
uuuggcaaugguagaacucaca

SEQ ID NO: 53 - RNA, 23 nt, Mus musculus
uauggcacugguagaauucacug

SEQ ID NO: 54 - RNA, 18 nt, Mus musculus
uggagagaaaggcaguuc

SEQ ID NO: 55 - RNA, 22 nt, Mus musculus
ucgugucuuguguugcagccgg

SEQ ID NO: 56 - RNA, 22 nt, Mus musculus
caacggaaucccaaaagcagcu

SEQ ID NO: 57 - RNA, 22 nt, Mus musculus
uguaacagcaacuccaugugga

SEQ ID NO: 58 - RNA, 21 nt, Mus musculus
uagcagcacagaaauauuggc

SEQ ID NO: 59 - RNA, 22 nt, Mus musculus
acaguagucugcacauugguua

SEQ ID NO: 60 - RNA, 23 nt, Mus musculus
cccaguguucagacuaccuguuc

SEQ ID NO: 61 - RNA, 23 nt, Mus musculus
cccaguguuuagacuaccuguuc

SEQ ID NO: 62 - RNA, 23 nt, Mus musculus
ugugcaaauccaugcaaaacuga

SEQ ID NO: 63 - RNA, 22 nt, Mus musculus
uaacacugucugguaacgaugu

SEQ ID NO: 64 - RNA, 23 nt, Mus musculus
uaauacugccugguaaugaugac

SEQ ID NO: 65 - RNA, 22 nt, Mus musculus
uaauacugccggguaaugaugg

SEQ ID NO: 66 - RNA, 21 nt, Mus musculus
ugaaauguuuaggaccacuag

SEQ ID NO: 67 - RNA, 22 nt, Mus musculus
uccuucauuccaccggagucug

SEQ ID NO: 68 - RNA, 22 nt, Mus musculus
uggaauguaaggaagugugugg

SEQ ID NO: 69 - RNA, 23 nt, Mus musculus
uaaagugcuuauagugcagguag

SEQ ID NO: 70 - RNA, 22 nt, Mus musculus
caaagugcucauagugcaggua

SEQ ID NO: 71 - RNA, 22 nt, Mus musculus
uagcuuaucagacugauguuga

SEQ ID NO: 72 - RNA, 21 nt, Mus musculus
acagcaggcacagacaggcag

SEQ ID NO: 73 - RNA, 21 nt, Mus musculus
uugugcuugaucuaaccaugu

SEQ ID NO: 74 - RNA, 22 nt, Mus musculus
aagcugccaguugaagaacugu

SEQ ID NO: 75 - RNA, 22 nt, Mus musculus
agcuacauugucugcuggguuu

SEQ ID NO: 76 - RNA, 24 nt, Mus musculus
agcuacaucuggcuacugggucuc

SEQ ID NO: 77 - RNA, 21 nt, Mus musculus
ugucaguuugucaaauacccc

SEQ ID NO: 78 - RNA, 23 nt, Mus musculus
uaagucacuagugguuccguuua

SEQ ID NO: 79 - RNA, 21 nt, Mus musculus
aucacauugccagggauuucc

SEQ ID NO: 80 - RNA, 21 nt, Mus musculus
aucacauugccagggauuacc

SEQ ID NO: 81 - RNA, 22 nt, Mus musculus
uggcucaguucagcaggaacag

SEQ ID NO: 82 - RNA, 22 nt, Mus musculus
cauugcacuugucucggucuga

SEQ ID NO: 83 - RNA, 21 nt, Mus musculus
uucaaguaauccaggauaggc

SEQ ID NO: 84 - RNA, 22 nt, Mus musculus
uucaaguaauucaggauagguu

SEQ ID NO: 85 - RNA, 21 nt, Mus musculus
uucacaguggcuaaguuccgc

SEQ ID NO: 86 - RNA, 21 nt, Mus musculus
uucacaguggcuaaguucugc

SEQ ID NO: 87 - RNA, 22 nt, Mus musculus
aaggagcucacagucuauugag

SEQ ID NO: 88 - RNA, 21 nt, Mus musculus
uagcaccaucugaaaucgguu

SEQ ID NO: 89 - RNA, 23 nt, Mus musculus
uagcaccauuugaaaucaguguu

SEQ ID NO: 90 - RNA, 20 nt, Mus musculus
uagcaccauuugaaaucggu

SEQ ID NO: 91 - RNA, 22 nt, Mus musculus
cuuucagucggauguuugcagc

SEQ ID NO: 92 - RNA, 22 nt, Mus musculus
uguaaacauccucgacuggaag

SEQ ID NO: 93 - RNA, 22 nt, Mus musculus
uguaaacauccuacacucagcu

SEQ ID NO: 94 - RNA, 23 nt, Mus musculus
uguaaacauccuacacucucagc

SEQ ID NO: 95 - RNA, 22 nt, Mus musculus
uguaaacauccccgacuggaag

SEQ ID NO: 96 - RNA, 20 nt, Mus musculus
uguaaacauccuugacugga

SEQ ID NO: 97 - RNA, 22 nt, Mus musculus
cuuucagucggauguuuacagc

SEQ ID NO: 98 - RNA, 22 nt, Mus musculus
aggcaagaugcuggcauagcug

SEQ ID NO: 99 - RNA, 23 nt, Mus musculus
aaaagcuggguugagagggcgaa

SEQ ID NO: 100 - RNA, 20 nt, Mus musculus
aaacaugaagcgcugcaaca

SEQ ID NO: 101 - RNA, 22 nt, Mus musculus
ccacugccccaggugcugcugg

SEQ ID NO: 102 - RNA, 22 nt, Mus musculus
cgcauccccuagggcauuggug

SEQ ID NO: 103 - RNA, 22 nt, Mus musculus
cuggcccucucugcccuuccgu

SEQ ID NO: 104 - RNA, 21 nt, Mus musculus
gccccugggccuauccuagaa

SEQ ID NO: 105 - RNA, 23 nt, Mus musculus
ucaagagcaauaacgaaaaaugu

SEQ ID NO: 106 - RNA, 21 nt, Mus musculus
ucgaucggucggucggucagu

SEQ ID NO: 107 - RNA, 24 nt, Mus musculus
ucucacacagaaaucgcacccguc

SEQ ID NO: 108 - RNA, 21 nt, Mus musculus
ugcugaccccuaguccagugc

SEQ ID NO: 109 - RNA, 23 nt, Mus musculus
uggcagugucuuagcugguuguu

SEQ ID NO: 110 - RNA, 23 nt, Mus musculus
aggcaguguaguuagcugauugc

SEQ ID NO: 111 - RNA, 23 nt, Mus musculus
uucacaaagcccauacacuuuca

SEQ ID NO: 112 - RNA, 24 nt, Mus musculus
ucccugaggagcccuuugagccug

SEQ ID NO: 113 - RNA, 22 nt, Mus musculus
uuaucagaaucuccagggguac

SEQ ID NO: 114 - RNA, 22 nt, Mus musculus
uaaugccccuaaaaauccuuau

SEQ ID NO: 115 - RNA, 22 nt, Mus musculus
auauaauacaaccugcuaagug

SEQ ID NO: 116 - RNA, 22 nt, Mus musculus
uuuguucguucggcucgcguga

SEQ ID NO: 117 - RNA, 21 nt, Mus musculus
ugguagacuauggaacguagg

SEQ ID NO: 118 - RNA, 22 nt, Mus musculus
cuggacuuggagucagaaggcc

SEQ ID NO: 119 - RNA, 22 nt, Mus musculus
cagcagcaauucauguuuugga

SEQ ID NO: 120 - RNA, 22 nt, Mus musculus
uaauacugucugguaaugccgu

SEQ ID NO: 121 - RNA, 21 nt, Mus musculus
uuugaaccaucacucgacucc

SEQ ID NO: 122 - RNA, 22 nt, Mus musculus
uggcaguguauuguuagcuggu

SEQ ID NO: 123 - RNA, 22 nt, Mus musculus
uuuuugcgauguguuccuaaua

SEQ ID NO: 124 - RNA, 22 nt, Mus musculus
aaaccguuaccauuacugaguu

SEQ ID NO: 125 - RNA, 24 nt, Mus musculus
augcaguccacgggcauauacacu

SEQ ID NO: 126 - RNA, 22 nt, Mus musculus
auauacauacacacaccuacac

SEQ ID NO: 127 - RNA, 22 nt, Mus musculus
auauacauacacacaccaacac

SEQ ID NO: 128 - RNA, 22 nt, Mus musculus
ucaggcucaguccccucccgau

SEQ ID NO: 129 - RNA, 22 nt, Mus musculus
uccuguacugagcugccccgag

SEQ ID NO: 130 - RNA, 22 nt, Mus musculus
cagcagcacacugugguuugua

SEQ ID NO: 131 - RNA, 22 nt, Mus musculus
aaugcacccgggcaaggauuug

SEQ ID NO: 132 - RNA, 22 nt, Mus musculus
caugccuugaguguaggaccgu

SEQ ID NO: 133 - RNA, 23 nt, Mus musculus
aagggauucugauguuggucaca

SEQ ID NO: 134 - RNA, 23 nt, Mus musculus
aauggcgccacuaggguugugca

SEQ ID NO: 135 - RNA, 22 nt, Mus musculus
auaguuguguguggaugugugu

SEQ ID NO: 136 - RNA, 24 nt, Mus musculus
aggaagcccuggaggggcuggagg

SEQ ID NO: 137 - RNA, 22 nt, Mus musculus
ugagguugguguacugugugug

SEQ ID NO: 138 - RNA, 22 nt, Mus musculus
gcacugagaugggaguggugua

SEQ ID NO: 139 - RNA, 22 nt, Mus musculus
cacagcucccaucucagaacaa

SEQ ID NO: 140 - RNA, 21 nt, Mus musculus
ccguccugagguuguugagcu

SEQ ID NO: 141 - RNA, 21 nt, Mus musculus
cguccccgcucggcggggucc

SEQ ID NO: 142 - RNA, 22 nt, Mus musculus
aaaggcuaggcucacaaccaaa

SEQ ID NO: 143 - RNA, 20 nt, Mus musculus
ggugggagguggggugggca

SEQ ID NO: 144 - RNA, 19 nt, Mus musculus
ggaggcagaggcaggagga

SEQ ID NO: 145 - RNA, 18 nt, Mus musculus
aucucgcuggggccucca

SEQ ID NO: 146 - RNA, 21 nt, Mus musculus
ugcggggcuagggcuaacagc

SEQ ID NO: 147 - RNA, 22 nt, Mus musculus
ggggcuggggccgggacagagc

SEQ ID NO: 148 - RNA, 20 nt, Mus musculus
gaauugaucaggacauaggg

SEQ ID NO: 149 - RNA, 21 nt, Mus musculus
uauugcacuugucccggccug

SEQ ID NO: 150 - RNA, 23 nt, Mus musculus
caaagugcuguucgugcagguag

SEQ ID NO: 151 - RNA, 22 nt, Mus musculus
ugagguaguaaguuguauuguu

SEQ ID NO: 152 - RNA, 20 nt, Mus musculus
acccguagauccgaucuugu

SEQ ID NO: 153 - RNA, 22 nt, Mus musculus
cacccguagaaccgaccuugcg

SEQ ID NO: 154 - RNA, 22 nt, Mus musculus
uacaguacugugauaacugaag

SEQ ID NO: 155 - RNA, 22 nt, Mus musculus
uacaguacugugauagcugaag

SEQ ID NO: 156 - RNA, 23 nt, Mus musculus
agcagcauuguacagggcuauga

SEQ ID NO: 157 - RNA, 23 nt, Mus musculus
agcagcauuguacagggcuauca

SEQ ID NO: 158 - RNA, 22 nt, Mus musculus
ugagaacugaauuccauggguu

SEQ ID NO: 159 - RNA, 22 nt, Mus musculus
ugagaacugaauuccauaggcu

SEQ ID NO: 160 - RNA, 22 nt, Mus musculus
ucagugcacuacagaacuuugu

SEQ ID NO: 161 - RNA, 22 nt, Mus musculus
ucagugcaucacagaacuuugu

SEQ ID NO: 162 - RNA, 22 nt, Mus musculus
ucagugcaugacagaacuuggg

SEQ ID NO: 163 - RNA, 22 nt, Mus musculus
uuaaugcuaauugugauagggg

SEQ ID NO: 164 - RNA, 23 nt, Mus musculus
aacauucaacgcugucggugagu

SEQ ID NO: 165 - RNA, 22 nt, Mus musculus
aacauucauugcugucgguggg

SEQ ID NO: 166 - RNA, 22 nt, Mus musculus
aacauucaaccugucggugagu

SEQ ID NO: 167 - RNA, 22 nt, Mus musculus
acaguagucugcacauugguua

SEQ ID NO: 168 - RNA, 23 nt, Mus musculus
cccaguguucagacuaccuguuc

SEQ ID NO: 169 - RNA, 23 nt, Mus musculus
cccaguguuuagacuaccuguuc

SEQ ID NO: 170 - RNA, 21 nt, Mus musculus
uucacaguggcuaaguuccgc

SEQ ID NO: 171 - RNA, 21 nt, Mus musculus
uucacaguggcuaaguucugc

SEQ ID NO: 172 - RNA, 20 nt, Mus musculus
ggugggagguggggugggca

SEQ ID NO: 173 - RNA, 19 nt, Mus musculus
ggaggcagaggcaggagga

SEQ ID NO: 174 - RNA, 22 nt, Mus musculus
ggggcuggggccgggacagagc

SEQ ID NO: 175 - RNA, 26 nt, Artificial Sequence
  Synthetic primer
guucagaguucuacaguccgacgauc

SEQ ID NO: 176 - DNA, 19 bp, Artificial Sequence
  Synthetic primer
ucguaugccgucuucugct

SEQ ID NO: 177 - DNA, 44 bp, Artificial Sequence
  Synthetic primer
aatgatacggcgaccaccgacaggttcagagttctacagtccga

SEQ ID NO: 178 - DNA, 21 bp, Artificial Sequence
  Synthetic primer
caagcagaagacggcatacga

SEQ ID NO: 179 - DNA, 31 bp, Mus musculus
gccgaggatccgtcgccatcaccttcttgat

SEQ ID NO: 180 - DNA, 30 bp, Mus musculus
cttagcggccgcctgagatcgccggaccac

SEQ ID NO: 181 - DNA, 34 bp, Mus musculus
gattagcggccgcccctcagtgtcccacagagta

SEQ ID NO: 182 - DNA, 32 bp, Mus musculus
caggctccatggtgcactctccccacacataa

SEQ ID NO: 183 - DNA, 31 bp, Mus musculus
gactaccatggcaacctggagggtttcatgt

SEQ ID NO: 184 - DNA, 32 bp, Mus musculus
caggctgaattcctcttcagaccctcccactc

SEQ ID NO: 185 - DNA, 20 bp, Artificial Sequence
  Synthetic primer
gcgacttcttcatccagagc

SEQ ID NO: 186 - DNA, 21 bp, Artificial Sequence
  Synthetic primer
ccggtgatggtcttcttccat

SEQ ID NO: 187 - DNA, 26 bp, Homo sapiens
gcattggtggttcagtggtagaattc

SEQ ID NO: 188 - DNA, 7 bp, Homo sapiens
cattggt

SEQ ID NO: 189 - DNA, 25 bp, Homo sapiens
ggatatgatgactgattacctgaga

SEQ ID NO: 190 - DNA, 7 bp, Homo sapiens
gatatga

SEQ ID NO: 191 - DNA, 20 bp, Homo sapiens
ctgaactaacaagtaacctt

SEQ ID NO: 192 - DNA, 7 bp, Homo sapiens
tgaacta

SEQ ID NO: 193 - DNA, 20 bp, Homo sapiens
agctgtcttgtagttccttc

SEQ ID NO: 194 - DNA, 7 bp, Homo sapiens
gctgtct

SEQ ID NO: 195 - DNA, 20 bp, Homo sapiens
taccactgagctacatcccc

SEQ ID NO: 196 - DNA, 7 bp, Homo sapiens
accactg

SEQ ID NO: 197 - DNA, 20 bp, Homo sapiens
taccactgagctacaccccc

SEQ ID NO: 198 - DNA, 7 bp, Homo sapiens
accactg

SEQ ID NO: 199 - DNA, 21 bp, Homo sapiens
tttcagcttaacaatcaatct

SEQ ID NO: 200 - DNA, 7 bp, Homo sapiens
ttcagct

SEQ ID NO: 201 - DNA, 21 bp, Homo sapiens
tcaccactataccaccaacgc

SEQ ID NO: 202 - DNA, 7 bp, Homo sapiens
caccact

SEQ ID NO: 203 - DNA, 20 bp, Homo sapiens
taccactgagctacatcccc

SEQ ID NO: 204 - DNA, 7 bp, Homo sapiens
accactg

SEQ ID NO: 205 - DNA, 22 bp, Homo sapiens
aggcttagagctataagcctct

SEQ ID NO: 206 - DNA, 7 bp, Homo sapiens
ggcttag

SEQ ID NO: 207 - DNA, 22 bp, Homo sapiens
acatatacatgctcacacttat

SEQ ID NO: 208 - DNA, 7 bp, Homo sapiens
catatac

SEQ ID NO: 209 - DNA, 22 bp, Homo sapiens
gtgggtagccaatggggtgctc

SEQ ID NO: 210 - DNA, 7 bp, Homo sapiens
tgggtag

SEQ ID NO: 211 - DNA, 20 bp, Homo sapiens
caggggccatgctaatcttc

SEQ ID NO: 212 - DNA, 7 bp, Homo sapiens
aggggcc

SEQ ID NO: 213 - DNA, 27 bp, Homo sapiens
tcaagatcaagcgagcttttgcccttc

SEQ ID NO: 214 - DNA, 7 bp, Homo sapiens
caagatc

SEQ ID NO: 215 - DNA, 22 bp, Homo sapiens
cgtcaaaaggttcatcatttgt

SEQ ID NO: 216 - DNA, 7 bp, Homo sapiens
gtcaaaa

SEQ ID NO: 217 - DNA, 21 bp, Homo sapiens
ctcgccgcccggccctcccgt

SEQ ID NO: 218 - DNA, 7 bp, Homo sapiens
tcgccgc

SEQ ID NO: 219 - DNA, 22 bp, Homo sapiens
atcctgtcttttgctacacatt

SEQ ID NO: 220 - DNA, 7 bp, Homo sapiens
tcctgtc

SEQ ID NO: 221 - DNA, 21 bp, Homo sapiens
gttcagttgttcaacaggtta

SEQ ID NO: 222 - DNA, 7 bp, Homo sapiens
ttcagtt

SEQ ID NO: 223 - DNA, 26 bp, Homo sapiens
gaattctaccactgaaccaccaatgc

SEQ ID NO: 224 - DNA, 7 bp, Homo sapiens
aattcta

SEQ ID NO: 225 - DNA, 23 bp, Homo sapiens
ataacgatgacgacgatgacgta

SEQ ID NO: 226 - DNA, 7 bp, Homo sapiens
taacgat

SEQ ID NO: 227 - DNA, 22 bp, Homo sapiens
ccaagccagccaggaacacata

SEQ ID NO: 228 - DNA, 7 bp, Homo sapiens
caagcca

SEQ ID NO: 229 - DNA, 22 bp, Homo sapiens
cgcccaattaatgtctgttgat

SEQ ID NO: 230 - DNA, 7 bp, Homo sapiens
gcccaat

SEQ ID NO: 231 - DNA, 22 bp, Homo sapiens
aaagccggagcccagaagtcgg

SEQ ID NO: 232 - DNA, 7 bp, Homo sapiens
aagccgg

SEQ ID NO: 233 - DNA, 22 bp, Homo sapiens
tggctctcctgcctgcaccacc

SEQ ID NO: 234 - DNA, 7 bp, Homo sapiens
ggctctc

SEQ ID NO: 235 - DNA, 22 bp, Homo sapiens
aaagctctctctccagtgtcca

SEQ ID NO: 236 - DNA, 7 bp, Homo sapiens
aagctct

SEQ ID NO: 237 - DNA, 22 bp, Homo sapiens
tgtccctgcctcctctgcccaa

SEQ ID NO: 238 - DNA, 7 bp, Homo sapiens
gtccctg

SEQ ID NO: 239 - DNA, 24 bp, Homo sapiens
ttcgcatcacccggatcagcagtc

SEQ ID NO: 240 - DNA, 7 bp, Homo sapiens
tcgcatc

SEQ ID NO: 241 - DNA, 20 bp, Artificial Sequence
  Synthetic primer
gggatgtagctcagtggtag

SEQ ID NO: 242 - DNA, 22 bp, Artificial Sequence
  Synthetic primer
gttggtggagcgatttgtctgg

SEQ ID NO: 243 - DNA, 21 bp, Artificial Sequence
  Synthetic primer
aagcagggtcgggcctggtta

SEQ ID NO: 244 - DNA, 23 bp, Artificial Sequence
  Synthetic primer
cttctgatcgaggcccagcccgt

SEQ ID NO: 245 - DNA, 20 bp, Artificial Sequence
  Synthetic primer
gggggtgtagctcagtggta

(19) United States
(12) Patent Application Publication
(10) Pub. No.: US 2013/0177624 A1
(43) Pub. Date: Jul. 11, 2013

(54) MIRNA EXPRESSION IN ALLERGIC DISEASE

(71) Applicants: David Brian Corry, Houston, TX (US); Farrah Kheradmand, Houston, TX (US); Preethi Gunaratne, Houston, TX (US); Sumanth Polikepahad, Houston, TX (US); Arash Olyie Naghavi, Katy, TX (US)

(72) Inventors: David Brian Corry, Houston, TX (US); Farrah Kheradmand, Houston, TX (US); Preethi Gunaratne, Houston, TX (US); Sumanth Polikepahad, Houston, TX (US); Arash Olyie Naghavi, Katy, TX (US)

(21) Appl. No.: 13/319,466

(22) PCT Filed: May 7, 2010
(86) PCT No.: PCT/US2010/034119

§ 371 Date: Oct. 1, 2012

Related U.S. Application Data
(60) Provisional application No. 61/176,824, filed on May 8, 2009.

(51) Int. Cl. C12N 15/113; A61K 31/713; A61K 45/06; C12Q 1/68

(52) U.S. Cl. CPC C12N 15/113; C12Q 1/6883; A61K 31/713; A61K 45/06
USPC 424/450; 506/16; 435/375; 514/44 A; 424/131.1; 435/6.12; 536/24.5; 435/6.13; 506/9

(57) ABSTRACT

Disclosed are methods for detecting an allergic lung disease that involve assessing the level of one or more microRNAs (miRNAs) in a biological sample, wherein the level of the one or more miRNAs in the biological sample compared to a reference level of the one or more miRNAs is indicative of allergic lung disease. Also disclosed are methods for the treatment or prevention of inflammatory or allergic lung disease that involve administration of a let-7 miRNA inhibitor as set forth herein, as well as biochips and kits that can be applied in the methods of the present invention.

This application claims priority to U.S. Application No. 61/176,824 filed on May 8, 2009, the entire disclosure of which is specifically incorporated herein by reference in its entirety without disclaimer.

This invention was made with government support under HL095382 and AI070973 awarded by the National Institutes of Health. The government has certain rights in the invention.

### BACKGROUND OF THE INVENTION

1. Field of the Invention

The present invention relates generally to the fields of molecular biology, immunology, and the diagnosis and treatment of allergic lung disease. More particularly, it concerns particular miRNA and their application in the diagnosis and treatment of allergic lung disease.

2. Description of Related Art

The allergic lung diseases comprise a clinically heterogeneous group of disorders that relative to other chronic diseases afflict a disproportionately large number of persons in highly industrialized societies. By far the most prevalent allergic lung disease is allergic asthma. Asthma affects approximately 1 out of 8 Americans including children, making it one of the most common of chronic ailments (Wills-Karp, 1999). Despite major advances in the understanding of asthma pathophysiology, prognosis and treatments have changed little over the past decade. Clinical signs and symptoms of asthma, including episodic dyspnea, cough, and shortness of breath, are due to airway obstruction, which in turn is related to airway hyperresponsiveness (AHR), a physiological alteration in which the airway transiently constricts in response to a wide variety of provocative stimuli (Wills-Karp, 1999).

Airway obstruction in asthma is frequently observed in the context of local and systemic allergic inflammation that may include elevated total allergen-specific immunoglobulin E (IgE) levels and increased numbers of eosinophils and T helper type 2 (Th₂) cells, a terminally differentiated CD4+ T cell that secretes interleukins 4 (IL-4), IL-5, IL-6, IL-9 and IL-13 (Fahy et al., 2000). Based on experimental studies of rodents, airway obstruction in the setting of allergic lung disease is largely mediated by the Th₂ cytokines IL-4 and IL-13 (Corry et al., 1996; Corry et al., 1998; Grunig et al., 1998). However, whereas IL-4 acts as a growth factor for Th₂ cells and immunoglobulin (Ig) E-secreting B cells, IL-13 acts on target lung tissues to induce AHR, goblet cell metaplasia and mucus hypersecretion (Corry, 1999). Despite the compartmentalized functional roles of IL-4 and IL-13, these cytokines are strongly related and signal through a common pathway that includes the alpha chain of the IL-4 receptor (IL-4Rα) (Zurawski et al., 1993), the alpha 1 chain of the IL-13 receptor (IL-13Rα1) (Hilton et al., 1996), the gamma chain of the IL-2 receptor (IL-2Rγ) (Russell et al., 1993; Matthews et al., 1995) and the transcription factor signal transducer and activator of transcription 6 (STAT6) (Hou et al., 1994).

Other signaling pathways are equally important to the coordinated generation of Th₂ cells and other allergic effector cells. The transcription factors GATA3 and STAT6 are essential for stable Th₂ cell commitment and Th₂ cytokine secretion (Zheng and Flavell, 1997; Kishikawa et al., 2001). In addition, co-stimulatory molecules are required for Th₂ cell development and experimental asthma, including tumor necrosis factor receptor super family-4 (Tnfrsf4; OX40) (Ohshima et al., 1998; Salek-Ardakani et al., 2003) and CD28 (Keane-Myers et al., 1997; McArthur and Raulet, 1993). Thus, asthma-like disease in mice is mediated through a final common Th2 cell-dependent IL-13 signaling pathway in which STAT6 is activated through IL-4Rα and IL-13Rα1. Moreover, Th₂ cells arise through the same cytokine signaling pathway activated by IL-4 and additionally through a variety of co-stimulatory signaling pathways. Numerous additional signaling circuits, including epidermal growth factor (Takeyama et al., 1999), thymic stromal lymphopoietin (Al-Shami et al., 2005; Zhou et al., 2005), IL-25 (Angkasekwinai et al., 2007), histaminergic (Dunford et al., 2006) and gamma amino butyric acid-(GABA)-ergic (Xiang et al., 2007) pathways, complement protein 3a (C3a) (Drouin et al., 2002), adenosine (Chunn et al., 2001) and many others complement these core signaling mechanisms and significantly modify expression of allergic inflammation and the allergic lung disease phenotype.

MicroRNAs (miRNAs) are short, non-coding RNAs that target and silence protein coding genes through 3′-UTR elements. Relatively few miRNAs have been studied and an overall understanding of the importance of these regulatory transcripts in complex in vivo systems is lacking. Further, the precise role of miRNA in a variety of biological and developmental functions has not been fully elucidated. Because a single miRNA can typically affect the expression of several hundred different transcripts, predicting the function or in vivo effect of even a single miRNA can be particularly challenging. Clearly, there is a need for new and/or improved methods for diagnosing and treating allergic lung disease.

### SUMMARY OF THE INVENTION

The present invention is based in part on the finding that substantial miRNA changes occur in the lung as a result of allergen challenge, and, further modulating or inhibiting the function of miRNA can result in the substantial inhibition of allergic and inflammatory responses in vivo. Specific miRNAs of potential disease relevance were observed to be down-regulated upon initial allergen challenge in a mouse model of allergic disease in humans. Certain aspects of the present invention are based in part on the finding of an additional layer of regulation involving post-transcriptional editing of multiple miRNAs that altered the target repertoire. As shown in the below examples, specific changes in miRNA expression were observed in the lung after allergen challenge in vivo. The inventors further discovered that inhibition of single or multiple let-7 miRNA, including, e.g., mmu-mir-155, markedly inhibited inflammatory and allergic lung disease indices in vivo. Various aspects of the present invention relate to therapeutically treating an inflammatory or allergic lung disease via the inhibition of one or more let-7 miRNA.

Certain aspects of the present invention are based, in part, on the discovery that let-7 miRNA affect allergic and inflammatory responses in the lung. Multiple technologies were applied to globally analyze miRNA expression and function in allergic lung disease, an experimental model of asthma. Deep sequencing and microarray analyses of the mouse lung short RNAome revealed numerous extant and novel miRNAs and other transcript classes. Similar to mRNAs, lung miRNA expression changed dynamically during the transition from the naïve to the allergic state, suggesting numerous functional relationships. A possible role for miRNA editing in altering the lung mRNA target repertoire was also identified. Multiple members of the highly conserved let-7 miRNA family were the most abundant lung miRNAs, and it was confirmed in vitro that interleukin 13 (IL-13), a cytokine essential for expression for allergic lung disease, is regulated by mmu-let-7a. However, inhibition of let-7 miRNAs in vivo using a locked nucleic acid (LNA) profoundly inhibited production of allergic cytokines and the disease phenotype. These findings thus reveal unexpected complexity in the miRNAome underlying allergic lung disease and demonstrate a pro-inflammatory role for let-7 miRNAs. While certain aspects of the present invention relate to miRNA changes in the lung, it is nonetheless anticipated that similar changes in miRNA expression may result from exposure to an allergen or inflammatory stimuli in other tissues, including, for example, the skin, gastrointestinal tract (including esophagus, stomach, intestine, and colon), upper respiratory tract (e.g., particularly the nasal sinuses), the eyes (e.g., particularly the corneas, sclerae and conjunctivae), liver and central nervous system (e.g., particularly the brain and spinal cord).

Some aspects of the present invention involve methods for detecting an allergic or inflammatory lung disease, comprising assessing the level of one or more microRNAs (miRNAs) in a biological sample, wherein the level of the one or more miRNAs in the biological sample compared to a reference level of the one or more miRNAs is indicative of allergic or inflammatory lung disease. In certain embodiments, at least one of the one or more miRNAs comprises: (i) mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, mir-712, Asth-miR-1, or Asth-miR-2; (ii) mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, or mir-467c; (iii) a sequence that has at least 80%, 81%, 82%, 83%, 84%, 85%, 86%, 87%, 88%, 89%, 90%, 91%, 92%, 93%, 94%, 95%, 96%, 97%, 98, or 99% sequence identity to a sequence as set forth in (i); (iv) a sequence that has at least 80%, 81%, 82%, 83%, 84%, 85%, 86%, 87%, 88%, 89%, 90%, 91%, 92%, 93%, 94%, 95%, 96%, 97%, 98, or 99% sequence identity to a sequence as set forth in (ii); (v) the complement of a sequence as set forth in (i) or (iii); or (vi) the complement of a sequence as set forth in (ii) or (iv); wherein a decrease in the expression level of one or more miRNAs from group (i), (iii) or (v), or an increase in the expression level of one or more miRNAs from group (ii), (iv) or (vi) in the biological sample compared to a reference level of the one or more miRNAs is indicative of allergic or inflammatory lung disease. The lung disease may be an allergic lung disease selected from the group consisting of asthma, hay fever, hypersensitivity pneumonitis, eosinophilic pneumonia (acute or chronic), Churg-Strauss Syndrome, allergic bronchopulmonary mycosis, and tropical eosinophilic pneumonia. In certain embodiments the allergic lung disease is asthma. The biological sample may comprise white blood cells or lung tissue. The method may further comprise obtaining a biological sample from a subject. In certain embodiments, more than one miRNAs is detected. In certain embodiments, the sequence of at least one miRNA is the complement of a sequence as set forth in (i) or (ii). At least one miRNA that is detected may or may not have a stem-loop structure. The method may further comprising detecting the presence or absence of one or more Piwi protein interacting RNAs (piRNAs).

Another aspect of the present invention relates to a biochip comprising an isolated nucleic acid comprising: (i) mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712; (ii) a sequence that has at least 80%, 81%, 82%, 83%, 84%, 85%, 86%, 87%, 88%, 89%, 90%, 91%, 92%, 93%, 94%, 95%, 96%, 97%, 98, or 99% sequence identity to a sequence as set forth in (i); (iii) the complement of a sequence as set forth in (i) or (ii); or (iv) a nucleic acid sequence comprising at least 10, 11, 12, 13, 14, 15, 16, 17, 18, 19, 20 or more contiguous nucleic acids of Asth-miR-1 (SEQ ID NO:187), Asth-miR 2 (SEQ ID NO:189), or Asth-miR-5 (SEQ ID NO:195); attached to said biochip. The biochip may comprise a plurality of nucleic acids as set forth in one or more of (i), (ii), (iii), and (iv).

Yet another aspect of the present invention involves methods of inhibiting a target gene in a cell, comprising contacting the cell with a nucleic acid in an amount sufficient to inhibit expression of the target gene, wherein the nucleic acid comprises: (i) mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712; (ii) a sequence that has at least 80%, 81%, 82%, 83%, 84%, 85%, 86%, 87%, 88%, 89%, 90%, 91%, 92%, 93%, 94%, 95%, 96%, 97%, 98, or 99% sequence identity to a sequence as set forth in (i); (iii) the complement of a sequence as set forth in (i) or (ii); or (iv) a nucleic acid sequence comprising at least 10, 11, 12, 13, 14, 15, 16, 17, 18, 19, 20 or more contiguous nucleic acids of Asth-miR-1 (SEQ ID NO:187), Asth-miR 2 (SEQ ID NO:189), or Asth-miR-5 (SEQ ID NO:195). The target gene may be an interleukin or a cytokine including, e.g., GATA3, STAT6, IL13RA1, GATA3, CD4, ADRB2, JAK1, IL4, JAK1, IRAK1, STAT6, or IL13. The cell may be in a subject, such as a mammal. In certain embodiments, the subject is a human. The human may or may not have an allergic lung disease or may or may not be suspected of having an allergic lung disease. The allergic lung disease may be asthma. The cell may be a lung cell.

Another aspect of the present invention relates to methods of treating or preventing exacerbation of an allergic lung disease in a subject, comprising administering to said subject a pharmaceutically effective amount of a composition comprising a nucleic acid comprising: (i) mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, mir-712, Asth-miR-1, or Asth-miR-2; or (ii) a nucleic acid which selectively binds or inhibits one or more of: mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, or mir-467c. The nucleic acid may be a group (ii) nucleic acid, and nucleic acid may be chemically modified or comprise a nucleotide analog. In certain embodiments, the nucleic acid is selected from the group consisting of (5′-AACTATACAACCTACTACCTCA-3′ (SEQ ID NO:246)), (5′-AACTATACAACCTCCTACCTCA-3′ (SEQ ID NO:247)), and (5′-CAACCTACTACCTC-3′ (SEQ ID NO:248)). The nucleic acid may be an LNA. The subject may be a mammal, such as a human. The allergic lung disease may be asthma, hay fever, or hypersensitivity pneumonitis. Said nucleic acid may comprise a phosphoramidate linkage, a phosphorothioate linkage, a phosphorodithioate linkage, or an O-methylphosphoroamidite linkage. Said nucleic acid may comprise one or more nucleotide analogs. In certain embodiments, the method further comprises administering to the subject one or more secondary forms of therapy for the treatment or prevention of allergic lung disease.

The therapeutic or preventive methods set forth herein may further involve administering to the subject one or more secondary forms of therapy for the treatment or prevention of allergic lung disease. Examples of secondary forms of therapy include a corticosteroid, a beta-2 adrenergic receptor agonist, a leukotrine modifier, an anti-immunoglobulin E (IgE) antibody, and a mast cell stabilizing agent.

The nucleic acid may optionally be included in a vector. For example, the vector may be a viral vector. Non-limiting examples of viral vectors include an adenovirus, an adeno-associated virus, a lentivirus, or a herpes virus. The vector may be a particular, such as a lipid-containing particle (e.g., liposome).

Administration of the pharmaceutical compositions of the present invention may be by any method known to those of ordinary skill in the art. Non-limiting examples include via an aerosol, topically, locally, intravenously, intraarterially, intramuscularly, by lavage, or by injection into the thoracic cavity.

Yet another aspect of the present invention relates to kits comprising a biochip as set forth herein and one or more sealed containers. The kit may further comprise instructions for use of said biochip.

Some aspects of the present invention relate to kits comprising a sealed container comprising a nucleic acid, wherein said nucleic acid comprises: (i) mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712; (ii) a sequence that has at least 80%, 81%, 82%, 83%, 84%, 85%, 86%, 87%, 88%, 89%, 90%, 91%, 92%, 93%, 94%, 95%, 96%, 97%, 98, or 99% sequence identity to a sequence as set forth in (i); (iii) the complement of a sequence as set forth in (i) or (ii); or (iv) a nucleic acid sequence comprising at least 10, 11, 12, 13, 14, 15, 16, 17, 18, 19, 20 or more contiguous nucleic acids of Asth-miR-1 (SEQ ID NO:187), Asth-miR 2 (SEQ ID NO:189), or Asth-miR-5 (SEQ ID NO:195). The kit may further comprise a set of primers specific for transcription or reverse transcription of one or more nucleic acid sequences as set forth in (i), (ii), (iii), or (iv). The kit may further comprise a biochip. The kti may further comprise instructions for use.

Yet another aspect of the present invention relates to kits comprising a sealed container comprising a set of primers specific for transcription or reverse transcription of a nucleic acid sequence, wherein said nucleic acid sequence comprises: (i) mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712; (ii) a sequence that has at least 80%, 81%, 82%, 83%, 84%, 85%, 86%, 87%, 88%, 89%, 90%, 91%, 92%, 93%, 94%, 95%, 96%, 97%, 98, or 99% sequence identity to a sequence as set forth in (i); (iii) the complement of a sequence as set forth in (i) or (ii); or (iv) a nucleic acid sequence comprising at least 10, 11, 12, 13, 14, 15, 16, 17, 18, 19, 20 or more contiguous nucleic acids of Asth-miR-1 (SEQ ID NO:187), Asth-miR 2 (SEQ ID NO:189), or Asth-miR-5 (SEQ ID NO:195).

Some aspects of the present invention relate to methods of treating an allergic or inflammatory lung disease in a subject comprising administering to the subject a let-7 miRNA inhibitor. In certain embodiments, the let-7 miRNA inhibitor is selected from the group consisting of siRNA, an antisense oligonucleotide, a locked nucleic acid (LNA), an antisense RNA, and a plasmid expressing an antisense RNA. The let-7 miRNA inhibitor may bind said miRNA under high stringency condiditons. In certain embodiments, the let-7 miRNA inhibitor is an LNA. In certain embodiments, the LNA comprises: (i) (5′-AACTATACAACCTACTACCTCA-3′ (SEQ ID NO:246)), (5′-AACTATACAACCTCCTACCTCA-3′ (SEQ ID NO:247)), or (5′-CAACCTACTACCTC-3′ (SEQ ID NO:248)); (ii) a sequence having at least 80% sequence identity to a sequence as set forth in (i); or (iii) the complement of a sequence as set forth in (i) or (ii). The let-7 miRNA inhibitor may be administered in a pharmaceutically acceptable composition. In certain embodiments, the let-7 miRNA inhibitor is administered orally, intravenously, via an aerosol, topically, locally, intravenously, intraarterially, intramuscularly, by lavage, or by injection into the thoracic cavity. The subject may be a mouse, a rat, a rodent, a cat, a horse, a goat, a sheep, a cow, a rabbit, a primate, or a human.

Yet another aspect of the present invention relates to an isolated nucleic acid comprising: (i) (5′-AACTATACAACCTACTACCTCA-3′, SEQ ID NO:246), (5′-AACTATACAACCTCCTACCTCA-3′ SEQ ID NO:247), or (5′-CAACCTACTACCTC-3′ SEQ ID NO:248); (ii) a sequence having at least 80% sequence identity to (5′-AACTATACAACCTACTACCTCA-3′ SEQ ID NO:246), (5′-AACTATACAACCTCCTACCTCA-3′ SEQ ID NO:247), or (5′-CAACCTACTACCTC-3′ SEQ ID NO:248); or (iii) the complement of a sequence as set forth in (i) or (ii); wherein the isolated nucleic acid can selectively bind a let-7 miRNA. In certain embodiments, the isolated nucleic acid selectively binds the let-7 miRNA under high stringency conditions. The nucleic acid may comprise a phosphoramidate linkage, a phosphorothioate linkage, a phosphorodithioate linkage, or an O-methylphosphoroamidite linkage, or other chemical modification. The nucleic acid may comprise one or more nucleotide analogs. In certain embodiments, the nucleic acid is a locked nucleic acid (LNA). The nucleic acid may be comprised in a pharmaceutically acceptable composition.

Another aspect of the present invention relates to an isolated nucleic acid selected from the group consisting of SEQ ID NO:285-322, or a complement thereof. The nucleic acid may be present on a biochip or a microarray.

Some aspects of the present invention relate to methods of screening for a modulator of an allergic or inflammatory lung response comprising: (a) contacting a lung cell with a candidate substance; and (b) measuring the expression level of one or more microRNAs (miRNAs) in the lung cell; wherein at least one of the one or more miRNAs comprises: mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712, Asth-miR-1 (SEQ ID NO:187), Asth-miR 2 (SEQ ID NO:189), or Asth-miR-5 (SEQ ID NO:195); wherein an increase in the expression level of one or more of: mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, mir-712, Asth-miR-1, or Asth-miR-2 in the lung cell indicates that the modulator can inhibit an allergic or inflammatory lung response; and wherein a decrease in the expression level of one or more of: mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c in the lung cell indicates that the modulator can inhibit an allergic or inflammatory lung response.

Yet another aspect of the present invention relates to methods of identifying a subject to receive an inhibitor of an allergic or inflammatory lung response comprising: measuring the expression level of one or more microRNAs (miRNAs) in a lung cell from the subject; wherein at least one of the one or more miRNAs comprises: mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712, Asth-miR-1 (SEQ ID NO:187), Asth-miR 2 (SEQ ID NO:189), or Asth-miR-5 (SEQ ID NO:195); wherein an increase in the expression level of one or more of: mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, mir-712, Asth-miR-1, or Asth-miR-2 in the lung cell indicates that the subject may therapeutically benefit from said inhibitor; and wherein a decrease in the expression level of one or more of: mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c in the lung cell indicates that the subject may therapeutically benefit from said inhibitor. The subject may be a human. The method may further comprises a method of personalizing a therapy for an allergic or inflammatory lung disease. Said measuring may be performed in a plurality of subjects. The method further comprises a method of identifying a sub-population of patients to receive said inhibitor; for example, these embodiments may be useful for identifying a sub-population which may particularly benefit from a therapy to treat an allergic or inflammatory lung disease.

Another aspect of the present invention relates to a transgenic mouse comprising a mutation in a let-7 miRNA, wherein the mutation prevents the expression of the let-7 miRNA, and wherein the mouse exhibits a reduced susceptibility to an allergic lung response. In certain embodiments, the let-7 miRNA is mir-155 (mouse miRNA-155).

Yet another aspect of the present invention relates to a progeny mouse of the mouse of claim 72, wherein the progeny mouse comprises a mutation in a let-7 miRNA, wherein the mutation prevents the expression of the let-7 miRNA, and wherein the progeny mouse exhibits a reduced susceptibility to an allergic lung response.

The reference level is a reference level of miRNA expression from a different subject or group of subjects. The reference level may be a reference level of expression of any of the aforementioned miRNAs from a subject known to be affected by an allergic lung disease or from a subject known to not be affected with an allergic lung disease. For example, the reference level may be the level of expression of one or more of the aforementioned miRNA species in one or more subjects with severe asthma. In other embodiments, the reference level is the level of expression of one or more of the aforementioned miRNA species in one or more subjects without asthma.

The reference level can be obtained from a single subject or from a group of subjects. The reference level of miRNA expression can be determined using any method known to those of ordinary skill in the art, such as any of the methods discussed above and elsewhere in this description. In some embodiments, the reference level is an average level of expression of any of the aforementioned miRNA obtained from a cohort of subjects with an allergic lung disease. The reference level may be a single value of miRNA expression, or it may be a range of values of miRNA expression. The reference level may also be depicted graphically as an area on a graph.

The subject may be any subject, such as an avian, an amphibian, or a mammal. Non-limiting examples of mammals include mice, rats, dogs, cats, horses, goats, sheep, cows, rabbits, primates, and humans. In particular embodiments, the subject is a patient that is suspected of having an allergic lung disease.

In particular embodiments, the level of more than one miRNA is assessed. The level of miRNA can be assessed by any method known to those of ordinary skill in the art. Non-limiting examples for assessing expression of miRNA are discussed in greater detail below.

“Allergic lung disease” as used herein refers to any disease of the lung that is associated with presence of eosinophils in the lung. Non-limiting examples of allergic lung disease include asthma, hay fever, hypersensitivity pneumonitis, eosinophilic pneumonia (acute or chronic), Churg-Strauss Syndrome, allergic bronchopulmonary mycosis, and tropical eosinophilic pneumonia. In specific embodiments, the allergic disease is asthma. “Asthma” is a common disorder in which chronic inflammation of the bronchial tubes (bronchi) makes them swell or construct, narrowing the airways. Asthma involves only the bronchial tubes and does not affect the air sacs (alveoli) or the parenchyma of the lung. Airway constriction in asthma is due to three major processes acting on the bronchi: inflammation, bronchospasm, and mucus over-production. Various factors may precipitate an asthma attack in a subject, including allergies, infections, strong odors, fumes, and so forth.

“Biological sample” as used herein may mean a sample of biological tissue or fluid that comprises nucleic acids. Such samples include, but are not limited to, tissue or fluid isolated from subjects. Biological samples may also include sections of tissues such as biopsy and autopsy samples, frozen sections taken for histologic purposes, blood (such as white blood cells), plasma, serum, sputum, stool, tears, mucus, hair, and skin. Biological samples also include explants and primary and/or transformed cell cultures derived from animal or patient tissues. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used. Tissue, such as lung tissue is specifically contemplated as a biological sample. Lung tissue may be obtained by any method known to those of ordinary skill in the art, such as via bronchoscopy or obtained at the time of thoracotomy.

The nucleic acids and miRNAs set forth herein may optionally include one or more phosphoramidate linkages, phosphorothioate linkages, phosphorodithioate linkages, or O-methylphosphoroamidite linkages. The nucleic acid may optionally include one or more nucleotide analogs. Non-limiting examples are discussed in greater detail in the specification below.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIGS. 1A-D: Characterization and distribution of small RNAs in mouse lung. (FIG. 1A), Frequency of NGS-derived sequences as a function of nucleotide (nt) length. The 21-23 nt peak is typical for miRNAs. (FIG. 1B), Pie charts shows absolute numbers of sequenced transcripts from distinct lung RNA classes comparing allergen challenged to naïve mice. (FIG. 1C), Distribution of nucleotide modifications along the length of mature lung miRNAs comparing allergen challenged to naïve mice. (FIG. 1D) Editing of mmu-let-7a-1 as detected by NGS comparing allergen challenged to naïve lungs in which the ninth nucleotide of the seed sequence ‘U’ has been modified to ‘G’. *: indicates canonical mature sequence (SEQ ID NOS:328-348).

FIGS. 2A-D: Gene and miRNA expression profiling of allergen challenged and naïve mouse lungs. (FIG. 2A) Heat map of genes (mRNAs) induced or repressed (P<0.01, fold>1.5) in allergen challenged versus naïve lung. (FIG. 2B) Validation of gene microarray findings by quantitative RT-PCR for selected genes. (FIG. 2C) Heat map of miRNAs induced or repressed (P<0.01, fold>1.5) in allergen challenged versus naïve lung. (FIG. 2D) Validation of miRNA microarray findings by quantitative RT-PCR for selected miRNAs. Bar graph data are presented as means±SEM, N=3; *: P<0.05.

FIGS. 3A-D: Inverse expression of IL13 and let-7a suggests a functional association. (FIG. 3A) The let-7a target sequence in the IL13 3′UTR is conserved across mammalia (Targetscan 5.1 (SEQ ID NOS:349-355)). (FIG. 3B) Mature let-7a sequence folded onto the mouse IL-13 3′UTR target site and predicted minimum free energy (mfe) value. (FIG. 3C, FIG. 3D) Quantitative RT-PCR analysis of IL13 and IFN-γ (FIG. 3C) and mmu-let-7a (FIG. 3D) transcripts from in vitro cultured Th1 and Th2 cells. Data are presented as means±SEM, N=3; *: P<0.05.

FIGS. 4A-I: IL13 expression is suppressed by let-7a. (FIG. 4A, FIG. 4B) let-7a suppresses mouse and human IL-13 in HEK293T cells. HEK293T cells were transfected with plasmids containing firefly luciferase under the control of the mouse (FIG. 4A) or human (FIG. 4B) IL-13 3′UTR or control 3′UTR and simultaneously with plasmids expressing pre-mmu-miR-705, scrambled pre-miR, or pre-let-7a (39, 117 or 350 ng) as indicated. After 2 days, gene expression was quantitated as firefly relative light units after normalizing for transfection efficiency based on Renilla luciferase activity (firefly/Renilla). (FIG. 4C, FIG. 4D) Anti-let-7a rescues mouse IL13 expression. HEK293T cells were transfected simultaneously with mouse (FIG. 4C) or human (FIG. 4D) IL13 3′UTR and pre-mmu-let-7a plasmids as in (FIG. 4A) and additionally scrambled, irrelevant (anti-mir-705) or anti-let-7a locked nucleic acids (LNA; 5.8, 17.5 and 52.5 pmol). After 2 days, IL-13 expression was assessed as firefly/renilla relative light units. (FIGS. 4E-G) let-7a suppresses IL-13 gene expression in primary T cells. (FIG. 4E) Mouse splenic CD4+ T cells were electroporated with FITC-labeled anti-mmu-let-7a LNAs (black curve) or sham (red curve) and the efficiency of transfection was assessed by flow cytometry. Additional T cells were transfected with control or anti-let-7a LNA (80 and 240 pmol) and relative expression of let-7a (FIG. 4F) and IL13 (FIG. 4G) transcripts were determined by RT-qPCR 24 hours later. (FIG. 4H) Editing of let-7a to let-7e reduces efficiency of targeting of IL13. HEK293T cells were transfected with mouse IL13 3′UTR-containing luciferase plasmid as in (FIG. 4A) and either plasmids for expression of let-7a or edited let-7a (U→G) and either scrambled or anti-let7a (U→G) LNA as indicated and the effect on IL13 gene expression was assessed as relative light units. (FIG. 41) Pre-let7a (U→G) is fully processed to let-7e. RT-qPCR quantitation of let-7e or let-7a in HEK293T cells transfected with either pre-let-7a or pre-let-7a (U→G) expression plasmids. Data are presented as means±SEM, N=3 or 4 replicates/condition; *: P<0.05 for the indicated comparisons.

FIGS. 5A-E. Let-7 miRNAs are required for expression of allergic lung disease. (FIG. 5A) Protocol timeline for ovalbumin (OVA) immunization intraperitoneally (IP) and challenge intranasally (IN) and LNA administration intravenously (IV). (FIG. 5B) Anti-let-7 LNA suppresses T cell let-7 and IL-13 in vivo. RT-qPCR analysis of let-7a, IL-13 and IFN-γ transcripts in splenic CD4 T cells from mice treated under the indicated conditions. (FIG. 5C), Airway responsiveness as assessed by the change in respiratory system resistance (R_{RS}) in response to graded intravenous acetylcholine (Ach) challenge. *: P<0.05 relative to naïve or OVA or OVA+Control LNA groups. (FIG. 5D), Total bronchoalveolar lavage fluid (BALF) inflammatory cells (eosinophils, macrophages, neutrophils, lymphocytes, total cells). (FIG. 5E), Bronchoalveolar lavage fluid levels of the indicated cytokines *: P<0.05 for the indicated comparisons. Data are presented as means±SEM, N=5 mice per group.

FIGS. 6A-F: Novel miRNAs Asth-miR-1 and 2. Putative novel miRNAs discovered from illumina sequence data using the algorithm described in Methods. (FIG. 6A (SEQ ID NOS:356-372), FIG. 6B (SEQ ID NOS:373-401)), Sequences aligning with Asth-miR-1 from naïve and allergen challenged lung, respectively. The copy number of each sequence variant is shown at the end of the sequence. (FIG. 6C (SEQ ID NOS:402-417)) Predicted folded hairpin with mature Asth-miR-1 sequence marked in red. (FIG. 6D (SEQ ID NOS:418-422), FIG. 6E), Sequences aligning with Asth-miR-2 from naïve and allergen challenged lung (AC) with copy number of each sequence shown. (FIG. 6F) Folded hairpin and mature miRNA sequence marked in red. Pri-miRNA Asth-miR-2 is a single exon gene located in the intronic region of the mouse nucleolin gene.

FIG. 7: Novel miRNAs discovered from mouse T cells. Mature miRNA sequences are outlined in yellow and are depicted in the context of the putative pre-miRNA sequences. Criteria for determining new miRNAs are based on sequence, folding characteristics within the putative pre-miRNA and the minimum free energy (mfe) of the association (see Methods). Red and Blue arrows indicate putative Drosha/Pasha and Dicer cleavage sites, respectively (SEQ ID NOS:423-428).

FIGS. 8A-D: mmu-mir-155 is required for expression of allergic lung disease. Wild type and mir-155−/− mice were challenged intranasally over two weeks with an allergenic fungal proteinase (FP) or PBS and the effect on the asthma phenotype was determined. (FIG. 8A) airway responsivenessas assessed by the change in respiratory system resistance (RRS) in response to intravenous acetylcholine (Ach) challenge. (FIG. 8B) Total and differential cell counts in bronchoalveolar lavage (BAL) fluid for macrophages (mac), eosinophils (Eos), neutrophils (Neu) and lymphocytes (Lymph). (FIG. 8C) Total IL-4- and interferon gamma (IFN-)-secreting cells detected from whole lung. (FIG. 8D) Concentration of selected cytokines in BAL fluid.

FIG. 9. Distribution of Small RNAs in Helper T Cells. The number of reads that exclusively mapped to one or a combination of three databases, miRNAs, piRNAs from T cell subsets.

FIGS. 10A-B: FIG. 10A, Highly expressed miRNAs including let-7 series miRNAT_{H}1 cells show increased expression of mmu-let-7c which is involved in CD4+ cell activation (Cobb et al. 2007; Li et al., 2007). T_{H}2 cells show decreased expression of mmu-mir-181a which is involved in CD4+ T cell development (Cobb et al., 2007; Li et al.,). FIG. 10B, Highly expressed miRNAs excluding let-7 series miRNA. T_{H}1 cells show increased mmu-mir-101a. The putative gene targets are: STAT6, GATA3, CD38, IL-4Rα. T_{H}2 cells show increased expression of mmu-mir-199a. The putative gene targets are: STAT6, GATA3, IL-4Rα, ICOS. miRNA expression confirmed by qRT-PCR.

FIG. 11. Novel putative targets. Target Scan4.1 was used to determine the putative targets of the seed sequences the novel miRNAs. Novel miRNAs that target GATA3 in naïve and T_{H}1 cells were found. In T_{H}2 cells, BCL6 was a putative target. In addition many cell survival and apoptotic gene targets were identified in all of the subsets (SEQ ID NOS:429-434).

FIG. 12. Putative miRNA:mRNA Associations. Bioinformatics analysis of Illumina sequencing data and mRNA microarray chip (Illumina) of effector T cell subsets identify putative gene targets of sequenced mRNAs. Putative miRNA Regulation of Gilz in Helper T Cell Differentiation.

FIG. 13. miRNA Mechanism of Action. During normal T_{H}1 polarization T-bet, NFAT, NF-κIFN-γ are up-regulated, Gilz overexpression inhibits TCR/CD3-induced NF-KB activation and nuclear translocation (Ayroldi et al., 2001) and contributes to CD4+ commitment toward T_{H}2 phenotype (Camarlle et al., 2005). The T_{H}1-specific functional association data suggests the miRNA targeting Gilz suppress its function thus allowing T_{H}1 lineage commitment.

### DESCRIPTION OF ILLUSTRATIVE EMBODIMENTS

### A. Definitions

“Subject” as used herein may mean fish, amphibians, reptiles, birds, and mammals, such as mice, rats, rabbits, goats, cats, dogs, cows, apes and humans.

“Attached” or “immobilized” as used herein to refer to a nucleic acid proble and a solid support may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules.

“Differential expression” may mean qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

“Gene” used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Label” as used herein may mean a composition detectable by spectroscopic, photochemical, biochemical, immunochemical, chemical, or other physical means. For example, useful labels include .sup.32P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

“Nucleic acid” or “oligonucleotide” or “polynucleotide” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, NR.sub.2 or CN, wherein R is C.sub.1-C.sub.6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al. (2005); Soutschek et al. (2004); and U.S. Patent Publication No. 20050107325, which are incorporated herein by reference. Modified nucleotides and nucleic acids may also include locked nucleic acids (LNA), as described in U.S. Patent Publication No. 2002/0115080, U.S. Pat. No. 6,268,490, and U.S. Pat. No. 6,770,748, which are incorporated herein by reference. LNA nucleotides include a modified extra methylene “bridge” connecting the 2′ oxygen and 4′ carbon of the ribose ring. The bridge “locks” the ribose in the 3′-endo (North) conformation, which is often found in the A-form of DNA or RNA. LNA nucleotides can be mixed with DNA or RNA bases in the oligonucleotide whenever desired. Such oligomers are commercially available from companies including Exiqon (Vedbaek, Denmark). Additional modified nucleotides and nucleic acids are described in U.S. Patent Publication Nos. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

A nucleic acid may be used to therapeutically inhibit a let-7 miRNA. For example a nucleic acid comprising a sequence having at least 80%, 85%, 90%, 95%, or all of SEQ ID NO:246-248 may be used to inhibit the function of a let-7 miRNA in vitro or in vivo. As shown in the below examples, the inhibition of one or more let-7 miRNA (e.g., mmu-mir-155) is sufficient to substantially inhibit allergic or inflammatory lung responses in vivo.

For example, in certain embodiments a complementary nucleic acid, such as a modified nucleic acid or an LNA, may be used to bind or suppress the function of one or more let-7 miRNA. As shown in the below examples, full-length LNAs anti-complementary to let-7a (5′-AACTATACAACCTACTACCTCA-3′ (SEQ ID NO:246)) or let-7e (5′-AACTATACAACCTCCTACCTCA-3′ (SEQ ID NO:247)) may be used to inhibit the function of these let-7a or let-7e, respectively. A truncated anti-let-7a,b,c.d LNA (e.g., 5′-CAACCTACTACCTC-3′ (SEQ ID NO:248)) may be used in vitro or in vivo to bind or inhibit the function of multiple miRNA, such as multiple let-7 miRNA. In certain embodiments, a LNA may be administered to a subject, such as a mouse, rat, primate, or human subject, to inhibit the function of one or more miRNA. As shown in the below examples, inhibition of the function of one or more let-7 miRNA (e.g., mmu-mir-155, etc.) can result in a decrease in an inflammatory and/or allergic lung response. It is anticipated that the foregoing sequences do not need to be LNA; similar effect may be achieved using one or more of the foregoing sequences either alone or comprising one or more modification (e.g., to reduce in vivo degradation, improve pharmacokinetics, etc.).

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Stringent hybridization conditions” used herein may mean conditions under which a first nucleic acid sequence will hybridize to a second nucleic acid sequence, such as in a complex mixture of nucleic acids. Stringent conditions are sequence-dependent and will be different in different circumstances. Stringent conditions may be selected to be about 5-10.degree. C. lower than the thermal melting point (T.sub.m) for the specific sequence at a defined ionic strength pH. The T.sub.m may be the temperature (under defined ionic strength, pH, and nucleic concentration) at which 50% of the probes complementary to the target hybridize to the target sequence at equilibrium (as the target sequences are present in excess, at T.sub.m, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30.degree. C. for short probes (e.g., about 10-50 nucleotides) and at least about 60.degree. C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5.times.SSC, and 1% SDS, incubating at 42.degree. C., or, 5.times.SSC, 1% SDS, incubating at 65.degree. C., with wash in 0.2.times.SSC, and 0.1% SDS at 65.degree. C.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

### B. MicroRNAs (miRNAs)

MicroRNAs (miRNAs) are short, non-coding RNAs that target and silence protein coding genes through 3′-UTR elements. Important roles for miRNAs in numerous biological processes have been established, but comprehensive analyses of miRNA function in complex diseases are lacking mRNAs are initially transcribed as primary miRNAs (pri-miRNAs) that are then cleaved by the nuclear RNAses Drosha and Pasha to yield precursor-miRNAs (pre-miRNAs). These precursors are further processed by the cytoplasmic RNAse III dicer to form short double stranded miR-miR* duplexes, one strand of which (miR) is then integrated into the RNA Induced Silencing Complex (RISC) that includes the enzymes dicer and Argonaute (Ago). The mature miRNAs (˜17-24 nt) direct RISC to specific target sites located within the 3′UTR of target genes. Once bound to target sites, miRNAs represses translation through mRNA decay, translational inhibition and/or sequestration into processing bodies (P-bodies) (Eulalio et al., 2008; Behm-Ansmant et al., 2006; Chu and Rana, 2006). Recent estimates find that over 60% of protein coding genes carry 3′-UTR miRNA target sites (Friedman et al., 2009). In this regard, miRNAs act as key regulators of processes as diverse as early development (Reinhart et al., 2000), cell proliferation and cell death (Brennecke et al., 2003), apoptosis and fat metabolism (Xu et al., 2003), and cell differentiation (Chen, 2004; Dostie et al., 2003). In addition, studies of miRNA expression in chronic lymphocytic leukemia (Calin et al., 2008), colonic adenocarcinoma (Michael et al., 2003), Burkitt's lymphoma (Metzler et al., 2004), cardiac disease (Zhao et al., 2007) and viral infection (Pfeffer et al., 2004) suggest vital links between miRNA and numerous diseases.

MicroRNAs are highly conserved during evolution and yet subjected to post-transcriptional modification through RNA editing. RNA-dependent adenosine deaminase (ADAR)-mediated A-to-I editing has been shown to mediate nucleotide changes in some pre-miRNAs (Habig et al., 2007; Bass, 2006). It has been previously shown that non-random nucleotide changes occurring in mouse ovary and pancreas miRNAs are enriched in nucleotides at the extreme 5′ end and at nucleotide 9 (Reid et al., 2008). U-to-G modifications at position 9 in mmu-let-7a can potentially modulate duplex stability and therefore regulate mRNA cleavage and decay (Reid et al., 2008). Thus, transcriptional and post-transcriptional regulatory processes potentially powerfully influence the regulatory potential of miRNAs.

miRNAs thus far observed have been approximately 21-22 nucleotides in length and they arise from longer precursors, which are transcribed from non-protein-encoding genes. See review of Carrington et al. (2003). The precursors form structures that fold back on each other in self-complementary regions; they are then processed by the nuclease Dicer in animals or DCL1 in plants. miRNA molecules interrupt translation through precise or imprecise base-pairing with their targets.

miRNAs are involved in gene regulation. Some miRNAs, including lin-4 and let-7, inhibit protein synthesis by binding to partially complementary 3′ untranslated regions (3′UTRs) of target mRNAs. Others function like siRNA and bind to perfectly complementary mRNA sequences to destroy the target transcript.

Research on microRNAs is increasing as scientists are beginning to appreciate the broad role that these molecules play in the regulation of eukaryotic gene expression. The two best understood miRNAs, lin-4 and let-7, regulate developmental timing in C. elegans by regulating the translation of a family of key mRNAs (reviewed in Pasquinelli, 2002). Several hundred miRNAs have been identified in C. elegans, Drosophila, mouse, and humans. As would be expected for molecules that regulate gene expression, miRNA levels have been shown to vary between tissues and developmental states. In addition, one study shows a strong correlation between reduced expression of two miRNAs and chronic lymphocytic leukemia, providing a possible link between miRNAs and cancer (Calin, 2002). Although the field is still young, there is speculation that miRNAs could be as important as transcription factors in regulating gene expression in higher eukaryotes.

There are a few examples of miRNAs that play critical roles in cell differentiation, early development, and cellular processes like apoptosis. lin-4 and let-7 both regulate passage from one larval state to another during C. elegans development (Ambros, 2003). mir-14 and bantam are drosophila miRNAs that regulate cell death, apparently by regulating the expression of genes involved in apoptosis (Brennecke et al., 2003, Xu et al., 2003). miR-181 guides hematopoietic cell differentiation (Chen et al., 2004). Enhanced understanding of the functions of miRNAs will undoubtedly reveal regulatory networks that contribute to normal development, differentiation, inter- and intra-cellular communication, cell cycle, angiogenesis, apoptosis, and many other cellular processes.

Certain embodiments of the present invention involve methods for diagnosing or treating an allergic lung disease in a subject that involves inhibiting the function or measuring expression, respectively, of one or more miRNA species in a sample from the subject. miRNA function can be inhibited, for example, by the administration of a complementary or substantially complementary nucleic acid (e.g., a modified nucleic acid such as LNA, etc.). The miRNA species that may be used to diagnose or treat an allergic or inflammatory lung disease include species selected from the group shown in Table 1 below.

TABLE 1
Selected miRNA

| miRNA | Sequence | SEQ.ID NO. |
|---|---|---|
| let-7a | UGAGGUAGUAGGUUGUAUAGU | SEQ.ID NO. 1 |
| let-7b | UGAGGUAGUAGGUUGUGUGGUU | SEQ.ID NO. 2 |
| let-7c | UGAGGUAGUAGGUUGUAUGGUU | SEQ.ID NO. 3 |
| let-7d | AGAGGUAGUAGGUUGCAUAGU | SEQ.ID NO. 4 |
| let-7d-3p | CUAUACGACCUGCUGCCUUUCU | SEQ.ID NO. 5 |
| let-7e | UGAGGUAGGAGGUUGUAUAGU | SEQ.ID NO. 6 |
| let-7f | UGAGGUAGUAGGUUGUAUAGU | SEQ.ID NO. 7 |
| let-7g | UGAGGUAGUAGUUUGUACAGU | SEQ.ID NO. 8 |
| let-7i | UGAGGUAGUAGUUUGUGCUGU | SEQ.ID NO. 9 |
| miR-1 | UGGAAUGUAAAGAAGUAUGUA | SEQ.ID NO. 10 |
| miR-100 | AACCCGUAGAUCCGAACUUGUG | SEQ.ID NO. 11 |
| mir-101a | UACAGUACUGUGAUAACUGAAG | SEQ.ID NO. 12 |
| mir-101b | UACAGUACUGUGAUAGCUGAAG | SEQ.ID NO. 13 |
| mir-103 | AGCAGCAUUGUACAGGGCUAUGA | SEQ.ID NO. 14 |
| miR-106a | CAAAGUGCUAACAGUGCAGGUA | SEQ.ID NO. 15 |
| miR-106b | UAAAGUGCUGACAGUGCAGAU | SEQ.ID NO. 16 |
| mir-107 | AGCAGCAUUGUACAGGGCUAUCA | SEQ.ID NO. 17 |
| miR-10a | UACCCUGUAGAUCCGAAUUUGUG | SEQ.ID NO. 18 |
| miR-10b | CCCUGUAGAACCGAAUUUGUGU | SEQ.ID NO. 19 |
| miR-125a | UCCCUGAGACCCUUUAACCUGUG | SEQ.ID NO. 20 |
| miR-125b | UCCCUGAGACCCUAACUUGUGA | SEQ.ID NO. 21 |
| miR-126-3p | UCGUACCGUGAGUAAUAAUGC | SEQ.ID NO. 22 |
| mir-126-5p | CAUUAUUACUUUUGGUACGCG | SEQ.ID NO. 23 |
| miR-127 | UCGGAUCCGUCUGAGCUUGGC | SEQ.ID NO. 24 |
| miR-128a | UCACAGUGAACCGGUCUCUUUU | SEQ.ID NO. 25 |
| miR-128b | UCACAGUGAACCGGUCUCUUUC | SEQ.ID NO. 26 |
| miR-130a | CAGUGCAAUGUUAAAAGGGCAU | SEQ.ID NO. 27 |
| mir-132 | UAACAGUCUACAGCCAUGGUCG | SEQ.ID NO. 28 |
| miR-133a | UUGGUCCCCUUCAACCAGCUGU | SEQ.ID NO. 29 |
| miR-133b | UUGGUCCCCUUCAACCAGCUA | SEQ.ID NO. 30 |
| miR-140-3p | UACCACAGGGUAGAACCACGG | SEQ.ID NO. 31 |
| miR-141 | UAACACUGUCUGGUAAAGAUGG | SEQ.ID NO. 32 |
| miR-142-5p | CAUAAAGUAGAAAGCACUAC | SEQ.ID NO. 33 |
| mir-143 | UGAGAUGAAGCACUGUAGCUCA | SEQ.ID NO. 34 |
| miR-145 | GUCCAGUUUUCCCAGGAAUCCCUU | SEQ.ID NO. 35 |
| mir-146 | UGAGAACUGAAUUCCAUGGGUU | SEQ.ID NO. 36 |
| mir-146b | UGAGAACUGAAUUCCAUAGGCU | SEQ.ID NO. 37 |
| mir-148a | UCAGUGCACUACAGAACUUUGU | SEQ.ID NO. 38 |
| mir-148b | UCAGUGCAUCACAGAACUUUGU | SEQ.ID NO. 39 |
| miR-149 | UCUGGCUCCGUGUCUUCACUCC | SEQ.ID NO. 40 |
| mir-150 | UCUCCCAACCCUUGUACCAGUG | SEQ.ID NO. 41 |
| mir-151 | CUAGACUGAGGCUCCUUGAGG | SEQ.ID NO. 42 |
| mir-152 | UCAGUGCAUGACAGAACUUGGG | SEQ.ID NO. 43 |
| mir-155 | UUAAUGCUAAUUGUGAUAGGGG | SEQ.ID NO. 44 |
| miR-15a | UAGCAGCACAUAAUGGUUUGUG | SEQ.ID NO. 45 |
| miR-15b | UAGCAGCACAUCAUGGUUUACA | SEQ.ID NO. 46 |
| miR-16 | UAGCAGCACGUAAAUAUUGGCG | SEQ.ID NO. 47 |
| miR-17-5p | CAAAGUGCUUACAGUGCAGGUAGU | SEQ.ID NO. 48 |
| miR-181a | AACAUUCAACGCUGUCGGUGAGU | SEQ.ID NO. 49 |
| miR-181b | AACAUUCAUUGCUGUCGGUGGG | SEQ.ID NO. 50 |
| miR-181c | AACAUUCAACCUGUCGGUGAGU | SEQ.ID NO. 51 |
| miR-182 | UUUGGCAAUGGUAGAACUCACA | SEQ.ID NO. 52 |
| miR-183 | UAUGGCACUGGUAGAAUUCACUG | SEQ.ID NO. 53 |
| miR-185 | UGGAGAGAAAGGCAGUUC | SEQ.ID NO. 54 |
| miR-187 | UCGUGUCUUGUGUUGCAGCCGG | SEQ.ID NO. 55 |
| miR-191 | CAACGGAAUCCCAAAAGCAGCU | SEQ.ID NO. 56 |
| miR-194 | UGUAACAGCAACUCCAUGUGGA | SEQ.ID NO. 57 |
| miR-195 | UAGCAGCACAGAAAUAUUGGC | SEQ.ID NO. 58 |
| mir-199a-3p | ACAGUAGUCUGCACAUUGGUUA | SEQ.ID NO. 59 |
| mir-199a | CCCAGUGUUCAGACUACCUGUUC | SEQ.ID NO. 60 |
| mir-199b | CCCAGUGUUUAGACUACCUGUUC | SEQ.ID NO. 61 |
| miR-19b | UGUGCAAAUCCAUGCAAAACUGA | SEQ.ID NO. 62 |
| miR-200a | UAACACUGUCUGGUAACGAUGU | SEQ.ID NO. 63 |
| miR-200b | UAAUACUGCCUGGUAAUGAUGAC | SEQ.ID NO. 64 |
| miR-200c | UAAUACUGCCGGGUAAUGAUGG | SEQ.ID NO. 65 |
| miR-203 | UGAAAUGUUUAGGACCACUAG | SEQ.ID NO. 66 |
| miR-205 | UCCUUCAUUCCACCGGAGUCUG | SEQ.ID NO. 67 |
| miR-206 | UGGAAUGUAAGGAAGUGUGUGG | SEQ.ID NO. 68 |
| miR-20a | UAAAGUGCUUAUAGUGCAGGUAG | SEQ.ID NO. 69 |
| miR-20b | CAAAGUGCUCAUAGUGCAGGUA | SEQ.ID NO. 70 |
| miR-21 | UAGCUUAUCAGACUGAUGUUGA | SEQ.ID NO. 71 |
| miR-214 | ACAGCAGGCACAGACAGGCAG | SEQ.ID NO. 72 |
| miR-218 | UUGUGCUUGAUCUAACCAUGU | SEQ.ID NO. 73 |
| miR-22 | AAGCUGCCAGUUGAAGAACUGU | SEQ.ID NO. 74 |
| miR-221 | AGCUACAUUGUCUGCUGGGUUU | SEQ.ID NO. 75 |
| miR-222 | AGCUACAUCUGGCUACUGGGUCUC | SEQ.ID NO. 76 |
| miR-223 | UGUCAGUUUGUCAAAUACCCC | SEQ.ID NO. 77 |
| miR-224 | UAAGUCACUAGUGGUUCCGUUUA | SEQ.ID NO. 78 |
| miR-23a | AUCACAUUGCCAGGGAUUUCC | SEQ.ID NO. 79 |
| miR-23b | AUCACAUUGCCAGGGAUUACC | SEQ.ID NO. 80 |
| miR-24 | UGGCUCAGUUCAGCAGGAACAG | SEQ.ID NO. 81 |
| miR-25 | CAUUGCACUUGUCUCGGUCUGA | SEQ.ID NO. 82 |
| miR-26a | UUCAAGUAAUCCAGGAUAGGC | SEQ.ID NO. 83 |
| miR-26b | UUCAAGUAAUUCAGGAUAGGUU | SEQ.ID NO. 84 |
| mir-27a | UUCACAGUGGCUAAGUUCCGC | SEQ.ID NO. 85 |
| mir-27b | UUCACAGUGGCUAAGUUCUGC | SEQ.ID NO. 86 |
| miR-28 | AAGGAGCUCACAGUCUAUUGAG | SEQ.ID NO. 87 |
| miR-29a | UAGCACCAUCUGAAAUCGGUU | SEQ.ID NO. 88 |
| miR-29b | UAGCACCAUUUGAAAUCAGUGUU | SEQ.ID NO. 89 |
| miR-29c | UAGCACCAUUUGAAAUCGGU | SEQ.ID NO. 90 |
| miR-30a-3p | CUUUCAGUCGGAUGUUUGCAGC | SEQ.ID NO. 91 |
| miR-30a-5p | UGUAAACAUCCUCGACUGGAAG | SEQ.ID NO. 92 |
| miR-30b | UGUAAACAUCCUACACUCAGCU | SEQ.ID NO. 93 |
| miR-30c | UGUAAACAUCCUACACUCUCAGC | SEQ.ID NO. 94 |
| miR-30d | UGUAAACAUCCCCGACUGGAAG | SEQ.ID NO. 95 |
| miR-30e | UGUAAACAUCCUUGACUGGA | SEQ.ID NO. 96 |
| miR-30e-3p | CUUUCAGUCGGAUGUUUACAGC | SEQ.ID NO. 97 |
| miR-31 | AGGCAAGAUGCUGGCAUAGCUG | SEQ.ID NO. 98 |
| miR-320 | AAAAGCUGGGUUGAGAGGGCGAA | SEQ.ID NO. 99 |
| miR-322 | AAACAUGAAGCGCUGCAACA | SEQ.ID NO. 100 |
| miR-324-3p | CCACUGCCCCAGGUGCUGCUGG | SEQ.ID NO. 101 |
| miR-324-5p | CGCAUCCCCUAGGGCAUUGGUG | SEQ.ID NO. 102 |
| miR-328 | CUGGCCCUCUCUGCCCUUCCGU | SEQ.ID NO. 103 |
| miR-331 | GCCCCUGGGCCUAUCCUAGAA | SEQ.ID NO. 104 |
| miR-335 | UCAAGAGCAAUAACGAAAAAUGU | SEQ.ID NO. 105 |
| miR-341 | UCGAUCGGUCGGUCGGUCAGU | SEQ.ID NO. 106 |
| miR-342 | UCUCACACAGAAAUCGCACCCGUC | SEQ.ID NO. 107 |
| miR-345 | UGCUGACCCCUAGUCCAGUGC | SEQ.ID NO. 108 |
| miR-34a | UGGCAGUGUCUUAGCUGGUUGUU | SEQ.ID NO. 109 |
| miR-34c | AGGCAGUGUAGUUAGCUGAUUGC | SEQ.ID NO. 110 |
| miR-350 | UUCACAAAGCCCAUACACUUUCA | SEQ.ID NO. 111 |
| miR-351 | UCCCUGAGGAGCCCUUUGAGCCUG | SEQ.ID NO. 112 |
| miR-361 | UUAUCAGAAUCUCCAGGGGUAC | SEQ.ID NO. 113 |
| miR-365 | UAAUGCCCCUAAAAAUCCUUAU | SEQ.ID NO. 114 |
| miR-374-5p | AUAUAAUACAACCUGCUAAGUG | SEQ.ID NO. 115 |
| miR-375 | UUUGUUCGUUCGGCUCGCGUGA | SEQ.ID NO. 116 |
| miR-379 | UGGUAGACUAUGGAACGUAGG | SEQ.ID NO. 117 |
| miR-422b | CUGGACUUGGAGUCAGAAGGCC | SEQ.ID NO. 118 |
| miR-424 | CAGCAGCAAUUCAUGUUUUGGA | SEQ.ID NO. 119 |
| miR-429 | UAAUACUGUCUGGUAAUGCCGU | SEQ.ID NO. 120 |
| miR-434-3p | UUUGAACCAUCACUCGACUCC | SEQ.ID NO. 121 |
| miR-449 | UGGCAGUGUAUUGUUAGCUGGU | SEQ.ID NO. 122 |
| miR-450 | UUUUUGCGAUGUGUUCCUAAUA | SEQ.ID NO. 123 |
| miR-451 | AAACCGUUACCAUUACUGAGUU | SEQ.ID NO. 124 |
| miR-455-3p | AUGCAGUCCACGGGCAUAUACACU | SEQ.ID NO. 125 |
| miR-467a | AUAUACAUACACACACCUACAC | SEQ.ID NO. 126 |
| miR-467b | AUAUACAUACACACACCAACAC | SEQ.ID NO. 127 |
| miR-484 | UCAGGCUCAGUCCCCUCCCGAU | SEQ.ID NO. 128 |
| miR-486 | UCCUGUACUGAGCUGCCCCGAG | SEQ.ID NO. 129 |
| miR-497 | CAGCAGCACACUGUGGUUUGUA | SEQ.ID NO. 130 |
| miR-501-3p | AAUGCACCCGGGCAAGGAUUUG | SEQ.ID NO. 131 |
| miR-532 | CAUGCCUUGAGUGUAGGACCGU | SEQ.ID NO. 132 |
| miR-541 | AAGGGAUUCUGAUGUUGGUCACA | SEQ.ID NO. 133 |
| miR-652 | AAUGGCGCCACUAGGGUUGUGCA | SEQ.ID NO. 134 |
| miR-669c | AUAGUUGUGUGUGGAUGUGUGU | SEQ.ID NO. 135 |
| miR-671 | AGGAAGCCCUGGAGGGGCUGGAGG | SEQ.ID NO. 136 |
| miR-672 | UGAGGUUGGUGUACUGUGUGUG | SEQ.ID NO. 137 |
| miR-674 | GCACUGAGAUGGGAGUGGUGUA | SEQ.ID NO. 138 |
| miR-674-3p | CACAGCUCCCAUCUCAGAACAA | SEQ.ID NO. 139 |
| miR-676 | CCGUCCUGAGGUUGUUGAGCU | SEQ.ID NO. 140 |
| miR-689 | CGUCCCCGCUCGGCGGGGUCC | SEQ.ID NO. 141 |
| miR-690 | AAAGGCUAGGCUCACAACCAAA | SEQ.ID NO. 142 |
| mir-705 | GGUGGGAGGUGGGGUGGGCA | SEQ.ID NO. 143 |
| miR-709 | GGAGGCAGAGGCAGGAGGA | SEQ.ID NO. 144 |
| miR-720 | AUCUCGCUGGGGCCUCCA | SEQ.ID NO. 145 |
| miR-744 | UGCGGGGCUAGGGCUAACAGC | SEQ.ID NO. 146 |
| mir-762 | GGGGCUGGGGCCGGGACAGAGC | SEQ.ID NO. 147 |
| miR-805 | GAAUUGAUCAGGACAUAGGG | SEQ.ID NO. 148 |
| miR-92 | UAUUGCACUUGUCCCGGCCUG | SEQ.ID NO. 149 |
| miR-93 | CAAAGUGCUGUUCGUGCAGGUAG | SEQ.ID NO. 150 |
| miR-98 | UGAGGUAGUAAGUUGUAUUGUU | SEQ.ID NO. 151 |
| miR-99a | ACCCGUAGAUCCGAUCUUGU | SEQ.ID NO. 152 |
| miR-99b | CACCCGUAGAACCGACCUUGCG | SEQ.ID NO. 153 |
| mir-101a | UACAGUACUGUGAUAACUGAAG | SEQ ID. NO. 154 |
| mir-101b | UACAGUACUGUGAUAGCUGAAG | SEQ ID NO: 155 |
| mir-103 | AGCAGCAUUGUACAGGGCUAUGA | SEQ ID NO: 156 |
| mir-107 | AGCAGCAUUGUACAGGGCUAUCA | SEQ ID NO: 157 |
| mir-146 | UGAGAACUGAAUUCCAUGGGUU | SEQ ID NO: 158 |
| mir-146b | UGAGAACUGAAUUCCAUAGGCU | SEQ ID NO: 159 |
| mir-148a | UCAGUGCACUACAGAACUUUGU | SEQ ID NO: 160 |
| mir-148b | UCAGUGCAUCACAGAACUUUGU | SEQ ID NO: 161 |
| mir-152 | UCAGUGCAUGACAGAACUUGGG | SEQ ID NO: 162 |
| mir-155 | UUAAUGCUAAUUGUGAUAGGGG | SEQ ID NO: 163 |
| miR-181a | AACAUUCAACGCUGUCGGUGAGU | SEQ ID NO: 164 |
| miR-181b | AACAUUCAUUGCUGUCGGUGGG | SEQ ID NO: 165 |
| miR-181c | AACAUUCAACCUGUCGGUGAGU | SEQ ID NO: 166 |
| mir-199a-3p | ACAGUAGUCUGCACAUUGGUUA | SEQ ID NO: 167 |
| mir-199a | CCCAGUGUUCAGACUACCUGUUC | SEQ ID NO: 168 |
| mir-199b | CCCAGUGUUUAGACUACCUGUUC | SEQ ID NO: 169 |
| mir-27a | UUCACAGUGGCUAAGUUCCGC | SEQ ID NO: 170 |
| mir-27b | UUCACAGUGGCUAAGUUCUGC | SEQ ID NO: 171 |
| mir-705 | GGUGGGAGGUGGGGUGGGCA | SEQ ID NO: 172 |
| miR-709 | GGAGGCAGAGGCAGGAGGA | SEQ ID NO: 173 |
| mir-762 | GGGGCUGGGGCCGGGACAGAGC | SEQ ID NO: 174 |
| mir-147 | GUGUGCGGAAAUGCUUCUGCUA | SEQ ID NO: 249 |
| mir-135a | UAUGGCUUUUUAUUCCUAUGUGA | SEQ ID NO: 250 |
| mir-135b | UAUGGCUUUUCAUUCCUAUGUGA | SEQ ID NO: 251 |
| mir-683 | CCUGCUGUAAGCUGUGUCCUC | SEQ ID NO: 252 |
| mir-130b | CAGUGCAAUGAUGAAAGGGCAU | SEQ ID NO: 253 |
| mir-615-5p | GGGGGUCCCCGGUGCUCGGAUC | SEQ ID NO: 254 |
| mir-142-3p | UGUAGUGUUUCCUACUUUAUGGA | SEQ ID NO: 255 |
| mir-130b | CAGUGCAAUGAUGAAAGGGCAU | SEQ ID NO:256 |
| mir-18b | UAAGGUGCAUCUAGUGCUGUUAG | SEQ ID NO:257 |
| mir-340-5p | UUAUAAAGCAAUGAGACUGAUU | SEQ ID NO:258 |
| mir-501-5p | AAUCCUUUGUCCCUGGGUGAAA | SEQ ID NO:259 |
| mir-1191 | CAGUCUUACUAUGUAGCCCUA | SEQ ID NO:260 |
| mir-421 | AUCAACAGACAUUAAUUGGGCGC | SEQ ID NO:261 |
| mir-717 | CUCAGACAGAGAUACCUUCUCU | SEQ ID NO:262 |
| mir-467c | UAAGUGCGUGCAUGUAUAUGUG | SEQ ID NO:263 |
| mir-681 | CAGCCUCGCUGGCAGGCAGCU | SEQ ID NO:264 |
| mir-880 | UACUCCAUCCUCUCUGAGUAGA | SEQ ID NO:265 |
| mir-1190 | UCAGCUGAGGUUCCCCUCUGUC | SEQ ID NO:266 |
| mir-671-3p | UCCGGUUCUCAGGGCUCCACC | SEQ ID NO:267 |
| mir-1196 | AAAUCUACCUGCCUCUGCCU | SEQ ID NO:268 |
| mir-667 | UGACACCUGCCACCCAGCCCAAG | SEQ ID NO:269 |
| mir-452 | UGUUUGCAGAGGAAACUGAGAC | SEQ ID NO:270 |
| mir-483 | AAGACGGGAGAAGAGAAGGGAG | SEQ ID NO:271 |
| mir-743a | GAAAGACACCAAGCUGAGUAGA | SEQ ID NO:272 |
| mir-485 | AGAGGCUGGCCGUGAUGAAUUC | SEQ ID NO:273 |
| mir-770-5p | AGCACCACGUGUCUGGGCCACG | SEQ ID NO:274 |
| mir-483* | UCACUCCUCCCCUCCCGUCUU | SEQ ID NO:275 |
| mir-193 | AACUGGCCUACAAAGUCCCAGU | SEQ ID NO:276 |
| mir-296-5p | AGGGCCCCCCCUCAAUCCUGU | SEQ ID NO:277 |
| mir-715 | CUCCGUGCACACCCCCGCGUG | SEQ ID NO:278 |
| mir-712 | CUCCUUCACCCGGGCGGUACC | SEQ ID NO:279 |

As shown in the below examples, certain miRNA were observed to be upregulated in the lung in response to an inflammatory or allergic challenge (e.g., mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c), while other miRNA were observed to be downregulated in response to an inflammatory or allergic challenge (e.g., mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, mir-712).

### C. Methods for Analyzing Expression of miRNA and Gene Expression

Some embodiments of the methods of the present invention involve analysis of miRNA expression or gene expression. Methods for analyzing gene expression or expression of miRNA include, but are not limited to, methods based on hybridization analysis of polynucleotides, sequencing of polynucleotides, and analysis of protein expression such as proteomics-based methods. Commonly used methods for the quantification of mRNA expression in a sample include northern blotting and in situ hybridization (Parker and Barnes, 1999), RNAse protection assays (Hod, 1992), and PCR-based methods, such as reverse transcription polymerase chain reaction (RT-PCR) (Weis et al., 1992). In some embodiments, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

1. PCR-Based Methods

Gene expression or miRNA expression can be analyzed using techniques that employ PCR. PCR is useful to amplify and detect transcripts from a sample. RT-PCR is a sensitive quantitative method that can be used to compare mRNA levels in different samples (e.g., endomyocardial biopsy samples) to examine gene expression signatures.

To perform RT-PCR, mRNA is isolated from a sample. For example, total RNA may be isolated from a sample of lung tissue. mRNA may also be extracted, for example, from frozen or archived paraffin-embedded and fixed tissue samples. Methods for mRNA extraction are known in the art. See, e.g., Ausubel et al. (1997). Methods for RNA extraction from paraffin embedded tissues are disclosed, for example, in Rupp and Locker, 1987, and De Andres et al., 1995. Purification kits for RNA isolation from commercial manufacturers, such as Qiagen, can be used. Other commercially available RNA isolation kits include MasterPure™ Complete DNA and RNA Purification Kit (EPICENTRE™, Madison, Wis.), and, Paraffin Block RNA Isolation Kit (Ambion, Inc.). Total RNA from tissue samples can be also isolated using RNA Stat-60 (Tel-Test) or by cesium chloride density gradient centrifugation.

RNA is then reverse transcribed into cDNA. The cDNA is amplified in a PCR reaction. A variety of reverse transcriptases are known in the art. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

For quantitative PCR, a third oligonucleotide, or probe, is used to detect nucleotide sequence located between the two PCR primers. The probe is non-extendible by Taq DNA polymerase enzyme, and typically is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative analysis.

RT-PCR can be performed using commercially available equipment, such as an ABI PRISM 7700™ Sequence Detection System (Perkin-Elmer-Applied Biosystems, Foster City, Calif., USA), or Lightcycler® (Roche Molecular Biochemicals, Mannheim, Germany). Samples can be analyzed using a real-time quantitative PCR device such as the ABI PRISM 7700™ Sequence Detection System™

A variation of the RT-PCR technique is real time quantitative PCR, which measures PCR product accumulation through a dual-labeled fluorigenic probe, such as a TaqMan™ probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Gene expression may be examined using fixed, paraffin-embedded tissues as the RNA source or fresh tissue such as tissue obtained from a biopsy of pulmonary tissue. Examples of methods of examining expression in fixed, paraffin-embedded tissues, are described, for example, in Godfrey et al., 2000; and Specht et. al., 2001.

Another approach for gene expression analysis employs competitive PCR design and automated, high-throughput matrix-assisted laser desorption ionization time-of-flight (MALDI-TOF) MS detection and quantification of oligonucleotides. This method is described by Ding and Cantor, 2003. See also the MassARRAY-based gene expression profiling method, developed by Sequenom, Inc. (San Diego, Calif.).

Additional PCR-based techniques for gene expression analysis include, e.g., differential display (Liang and Pardee, 1992); amplified fragment length polymorphism (iAFLP) (Kawamoto et al., 1999); BeadArray™ technology (Illumina, San Diego, Calif.; Oliphant et al., 2002; Ferguson et al., 2000); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., 2001); and high coverage expression profiling (HiCEP) analysis (Fukumura et al., 2003).

2. Microarrays

Other techniques for examining gene expression in a sample involve use of microarrays. Microarrays permit simultaneous analysis of a large number of gene expression products. Typically, polynucleotides of interest are plated, or arrayed, on a microchip substrate. The arrayed sequences are then hybridized with nucleic acids (e.g., DNA or RNA) from cells or tissues of interest. The source of mRNA typically is total RNA. If the source of mRNA is lung tissue, mRNA can be extracted.

In various embodiments of the microarray technique, probes to at least 10, 25, 50, 100, 200, 500, 1000, 1250, 1500, or 1600 polynucleotides are immobilized on an array substrate. The probes can include DNA, RNA, copolymer sequences of DNA and RNA, DNA and/or RNA analogues, or combinations thereof.

In some embodiments, a microarray includes a support with an ordered array of binding (e.g., hybridization) sites for each individual polynucleotide of interest. The microarrays can be addressable arrays, such as positionally addressable arrays where each probe of the array is located at a known, predetermined position on the solid support such that the identity of each probe can be determined from its position in the array.

Each probe on the microarray can be between about 10-50,000 nucleotides in length. The probes of the microarray can consist of nucleotide sequences of any length. An array can include positive control probes, such as probes known to be complementary and hybridizable to sequences in the test sample, and negative control probes such as probes known to not be complementary and hybridizable to sequences in the test sample.

Methods for attaching nucleic acids to a surface are well-known in the art. Methods for immobilizing nucleic acids on glass are described (Schena et al, 1995; DeRisi Shalon et al., 1996). Techniques are known for producing arrays with thousands of oligonucleotides at defined locations using photolithographic techniques are described by Fodor et al., 1991; Pease et al., 1994; Lockhart et al., 1996; U.S. Pat. Nos. 5,578,832; 5,556,752; and 5,510,270). Other methods for making microarrays have been described. See, e.g., Maskos and Southern, 1992. Any type of array may be used in the context of the present invention.

3. Serial Analysis of Gene Expression (SAGE)

Gene expression or miRNA expression in samples may also be determined by serial analysis of gene expression (SAGE), which is a method that allows the simultaneous and quantitative analysis of a large number of gene transcripts, without the need of providing an individual hybridization probe for each transcript (see Velculescu et al., 1995; and Velculescu et al., 1997). Briefly, a short sequence tag (about 10-14 nucleotides) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of a population of transcripts can be quantitatively evaluated by determining the abundance of individual tags, and identifying the gene corresponding to each tag.

4. Protein Detection Methodologies

Immunohistochemical methods are also suitable for detecting the expression of the genes. Antibodies, most preferably monoclonal antibodies, specific for a gene product are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Proteomic methods can allow examination of global changes in protein expression in a sample. Proteomic analysis may involve separation of individual proteins in a sample by 2-D gel electrophoresis (2-D PAGE), and identification of individual proteins recovered from the gel, such as by mass spectrometry or N-terminal sequencing, and analysis of the data using bioinformatics. Proteomics methods can be used alone or in combination with other methods for evaluating gene expression.

In various aspects, the expression of certain genes in a sample is detected to provide clinical information, such as information regarding prognosis. Thus, gene expression assays include measures to correct for differences in RNA variability and quality. For example, an assay typically measures and incorporates the expression of certain normalizing genes, such known housekeeping genes. Alternatively, normalization can be based on the mean or median signal (Ct) of all of the assayed genes or a large subset thereof (global normalization approach). In some embodiments, a normalized test RNA (e.g., from a patient sample) is compared to the amount found in a sample from a patient with left ventricular dysfunction. The level of expression measured in a particular test sample can be determined to fall at some percentile within a range observed in reference sets.

### D. Kits

The technology herein includes kits for evaluating miRNA or gene expression in samples. A “kit” refers to a combination of physical elements. For example, a kit may include, for example, one or more components such as probes, including without limitation specific primers, antibodies, a protein-capture agent, a reagent, an instruction sheet, and other elements useful to practice the technology described herein. These physical elements can be arranged in any way suitable for carrying out the invention.

Kits for analyzing RNA expression may include, for example, a set of oligonucleotide probes for detecting expression of a gene or a miRNA (e.g., from Table 1). The probes can be provided on a solid support, as in an array (e.g., a microarray), or in separate containers. The kits can include a set of oligonucleotide primers useful for amplifying a set of genes described herein, such as to perform PCR analysis. Kits can include further buffers, enzymes, labeling compounds, and the like. Any of the compositions described herein may be comprised in a kit. In a non-limiting example, an individual miRNA is included in a kit. The kit may further include water and hybridization buffer to facilitate hybridization of the two strands of the miRNAs. The kit may also include one or more transfection reagents to facilitate delivery of the miRNA to cells.

A kit for analyzing protein expression can include specific binding agents, such as immunological reagents (e.g., an antibody) for detecting protein expression of a gene of interest. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, such as a sterile aqueous solution.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale.

Such kits may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used for the manipulation or characterization of miRNA.

### E. Vectors for Cloning, Gene Transfer and Expression

Within certain embodiments expression vectors are employed to express a nucleic acid of interest, such as a miRNA that inhibits the expression of a particular gene. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

1. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 by of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 by apart before activity begins to decline. Depending on the promoter, it appears that individual elements can function either co-operatively or independently to activate transcription.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler |
| | et al., 1987; Weinberger et al., 1984; Kiledjian |
| | et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson |
| | et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester | Palmiter et al., 1982; |
| | (TFA) | Haslinger et al., 1985; |
| | Heavy metals | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse mammary | Glucocor- | Huang et al., 1981; Lee |
| tumor virus) | ticoids | et al., 1981; Majors et al., |
| | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 | Taylor et al., 1989, 1990a, |
| | Large T | 1990b |
| | Antigen | |
| Proliferin | Phorbol Ester- | Mordacq et al., 1989 |
| | TPA | |
| Tumor Necrosis Factor | PMA | Hensel et al., 1989 |
| Thyroid Stimulating | Thyroid | Chatterjee et al., 1989 |
| Hormone α Gene | Hormone | |

Of particular interest are muscle specific promoters, and more particularly, cardiac specific promoters. These include the myosin light chain-2 promoter (Franz et al., 1994; Kelly et al., 1995), the alpha actin promoter (Moss et al., 1996), the troponin 1 promoter (Bhaysar et al., 1996); the Na⁺/Ca²⁺ exchanger promoter (Barnes et al., 1997), the dystrophin promoter (Kimura et al., 1997), the alpha7 integrin promoter (Ziober and Kramer, 1996), the brain natriuretic peptide promoter (LaPointe et al., 1996) and the alpha B-crystallin/small heat shock protein promoter (Gopal-Srivastava, 1995), alpha myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

2. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

3. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

4. Delivery of Expression Vectors

There are a number of ways in which expression vectors may introduced into cells. In certain embodiments of the invention, the expression construct comprises a virus or engineered construct derived from a viral genome. One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991).

The adenovirus may be replication-defective or replication-competent. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984), lentivirus, and herpesviruses may be employed.

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kasp a et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, the oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### F. Clinical Information

### 1. Definitions

“Treatment” and “treating” as used herein refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of asthma.

A “sample” is any biological material obtained from an individual. For example, a “sample” may be a blood sample or a lung tissue sample.

### 2. Dosage

A pharmaceutically effective amount of a therapeutic agent as set forth herein is determined based on the intended goal, for example inhibition of cell death. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

For example, a dose of the therapeutic agent may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. Dosages of nucleic acid or LNA which may be used include, for example, about from 10-100 mg (LNA or nucleic acid)/g body weight, about 25-75 mg (LNA or nucleic acid)/g body weight, about mg (LNA or nucleic acid)/g body weight, or any range derivable therein. A dosage of about 50 mg (LNA or nucleic acid)/g mouse body weight was observed to be effective to substantially inhibit allergic or inflammatory lung responses in mice in vivo.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, it may be desirable to provide a continuous supply of a pharmaceutical composition to the patient. This could be accomplished by catheterization, followed by continuous administration of the therapeutic agent. The administration could be intra-operative or post-operative.

### G. Pharmaceutical Compositions and Routes for Administration to Patients

Some embodiments of the present invention involve administration of pharmaceutical compositions. Where clinical applications are contemplated, pharmaceutical compositions will be prepared in a form appropriate for the intended application. Generally, this will involve preparing compositions that are essentially free of pyrogens, as well as other impurities that could be harmful to humans or animals.

One will generally desire to employ appropriate salts and buffers in preparing compositions of therapeutic agents. Buffers also will be employed when recombinant cells are introduced into a patient. Aqueous compositions of the present invention comprise an effective amount of the therapeutic agent, dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the therapeutic agents of the compositions.

The active compositions of the present invention may include classic pharmaceutical preparations. Administration of these compositions according to the present invention may be via any common route so long as the target tissue is available via that route. Administration may be by any method known to those of ordinary skill in the art, such as intravenous, intradermal, subcutaneous, intramuscular, intraperitoneal or intrathecal injection, or by direct injection into cardiac tissue. Other modes of administration include oral, buccal, and nasogastric administration. The active compounds may also be administered parenterally or intraperitoneally. Such compositions would normally be administered as pharmaceutically acceptable compositions, as described supra. In particular embodiments, the composition is administered to a subject using a drug delivery device. For example, the drug delivery device may be a catheter or syringe. In some embodiments, the composition is applied as a coating to a medical device, such as a stent.

By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

For oral administration the therapeutic agents of the present invention generally may be incorporated with excipients. Any excipient known to those of ordinary skill in the art is contemplated.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### H. Combined Therapy

In another embodiment, it is envisioned to use an miRNA or an miRNA inhibitor as set forth herein in combination with other therapeutic modalities. Thus, in addition to the therapies described above, one may also provide to the patient more “standard” pharmaceutical cardiac therapies. Examples of other therapies include, without limitation, other pharmaceutical therapies of asthma or other allergic lung disease.

The other therapeutic modality may be administered before, concurrently with, or following administration of the miRNA The therapy using miRNA may precede or follow administration of the other agent(s) by intervals ranging from minutes to weeks. In embodiments where the other agent and the miRNA are administered separately, one would generally ensure that a significant period of time did not expire between the time of each delivery, such that each agent would still be able to exert an advantageously combined effect. In such instances, it is contemplated that one would typically administer the miRNA and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of an miRNA, or the other agent will be desired. In this regard, various combinations may be employed. By way of illustration, where the miRNA is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B
B/B/B/A A/A/A/B B/A/A/A A/B/A/A A/A/B/A A/B/B/B
B/A/B/B B/B/A/B

Other combinations are likewise contemplated. Non-limiting examples of pharmacological agents that may be used in the present invention include any pharmacological agent known to be of benefit in the treatment of asthma. Examples include inhaled corticosteroids, long-activing beta-2 agonists (such as salmetrol and formoterol), leukotriene modifiers such as montelukast, zafirlukast, and zileuton, cromolyn and nedocromil, theophylline, short-acting beta-2 agonists such as albuterol, ipratropium, and oral and intravenous corticosteroids. Further examples include immunotherapy and anti-IgE monoclonal antibodies, such as omalizumab.

### I. Biochips

A biochip is also provided. The biochip may comprise a solid substrate comprising an attached nucleic acid sequence that is capable of hybridizing to an miRNA sequence described herein. “Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder.

The probes may be attached to the biochip in a wide variety of ways, as will be appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography

### J. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Mice and Allergen Challenge.

All experiments were performed in accordance with institutional and United States National Institutes of Health guidelines. Allergen challenge of C57BL/6 mice was performed with an allergenic fungal proteinase and ovalbumin as previously described (Kheradmand et al., 2002).

Preparation of Short-RNA Transcripts for Illumina Sequencing.

Short RNA transcripts of <60 nucleotide length were gel purified after running 10 mg of total RNA on 15% TBE-Urea polyacrylamide gel. A synthetic 26-residue adapter RNA oligonucleotide (5′ GUU CAG AGU UCU ACA GUC CGA CGA UC 3′ (SEQ ID NO:280)) was ligated to the 5′ and of the small-RNAs. The ligated small-RNA was gel purified to remove un-ligated free adapter. A synthetic 22-residue 3′ adapter with inverted dideoxythymidine added at the 3′ end (5′ p UCG UAU GCC GUC UUC UGC UUG idT 3′ (SEQ ID NO:281)) was ligated to the 5′ ligated small-RNA and gel purified. The resultant RNA library was reverse transcribed and amplified by PCR for 15 cycles using adapter-specific primers. The PCR products were sequenced using Illumina (Solexa)-based Next Generation Sequencing.

Small RNA Mapping and Classification.

After filtering for the Illumina small RNA adapter sequences, the reads were mapped to the reference mouse genome (NCBI Build 37, UCSC mm9) using the Pash software package as previously described (Coarfa & Milosavljevic. 2008, Kalafus et al. 2004).

Novel miRNA Discovery.

All small RNA sequences that failed to align with a known miRNA, piRNA or snoRNA were passed through a novel miRNA discovery platform as described in Supplementary Experimental Procedures.

Microarray Analyses:

Illumina Sentrix Universal-12 Mouse v2 Gene Expression BeadChip Array (45281 transcripts) was used for gene profiling, and Illumina Mouse v2 MicroRNA Expression BeadChip Array (611 miRNAs) was used for miRNA profiling. The gene array data generated were quantile normalized (using software kindly provided by Dr. Kerby Shedden). Significantly regulated genes and miRNAs were identified by comparing allergen challenged with naïve using t-test (log-transformed data) and fold change (ratio of averages of the two groups). Java TreeView (Saldanha, 2004) represented expression patterns as color maps, where gene and miRNA values were centered on the median expression of the naïve group.

Isolation, Culture, and Transfection of CD4+ T Cells from Spleen.

Mouse spleens were collected and CD4 T cells isolated by immunomagnetic selection. Th1 and Th2 cells were differentiated as previously described (Grunig et al., 1998).

Nucleofection of in vitro anti-let-7a LNAs in to CD4 T cells was performed by using mouse T cell nucleofector kit (Lonza, Walkersville, Md.) according to the manufacturer's protocol and the cells were cultured for 48 hours. 80 and 240 pmol of anti-let-7a LNAs and 240 pmol of scrambled LNA were used for transfection. For determining the efficiency of transfection, cells that were transfected with flourescein labeled LNAs were nucleofected in to CD4 T cells and subjected to flow cytometry after 48 hours.

For RNA extraction, cells were homogenized in Trizol and total RNA was isolated using miRNeasy kit (Qiagen, Valencia, Calif.) according to the manufacturer's protocol.

In Vitro Validation.

HEK293T cells were used for co-transfection of plasmids expressing miRNAs, 3′UTR of target genes and anti-miRNA or control LNAs. Briefly, HEK293T cells that were cultured in 24-well plates were co-transfected with plasmids expressing IL-13 3′UTR (350-ng) or control 3′UTR (350-ng) and/or, mouse/human let-7a (350 or 117 or 39-ng) or mouse let-7a(U→G) (350-ng), or mouse miR-705 (350-ng) or scrambled miRNA (350-ng) and/or mouse/human anti-let-7a LNA (52.5, 17.5 and 5.8 pmol) or scrambled LNA (52.5 pmol) or anti-miR-705 LNA (52.5 pmol) or mouse anti-let-7e (let-7a (U→G)) LNA (52.5 pmol). Lipofectamine 2000 (Invitrogen, Carlsbad, Calif.) was used as transfection reagent according to the manufacturer's protocol. Firefly and Renilla Luciferase light units were measured after 2 days of co-transfection by using Dual-Luciferase Reporter Assay System (Promega, Madison, Wis.) with the help of FLOU star OPTIMA microplate reader (Bmg Labtech, Cary, N.C.).

In Vivo Transfection and Allergy Induction.

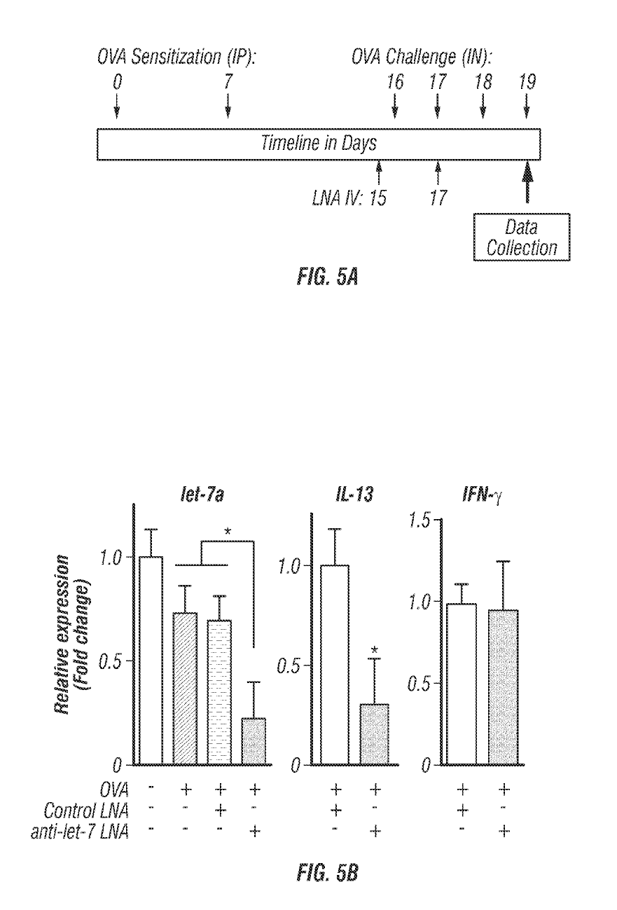

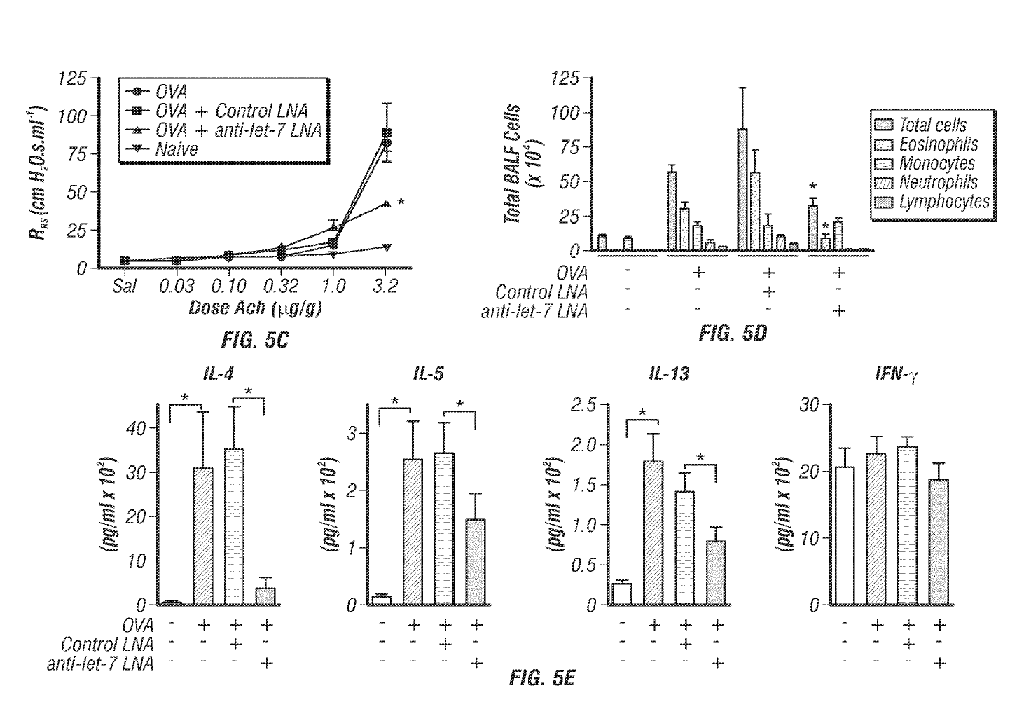

For in vivo LNA experiments, female Balb/c mice between 5-8 weeks were used. Mice were sensitized with 50 μL of chicken ovalbumin and alum by intraperitonial injection twice (day zero and 7) at one-week intervals. On days 15 and 17, LNAs prepared in 0.9% saline were injected intravenously into mice through the tail vein. On days 16, 17 and 18 mice were intranasally challenged with chicken ovalbumin (25 μg in 50 μL PBS) before analysis on day 19 (FIG. 5A).

Quantitation of Allergic Lung Disease.

24 hours after the final allergen challenge, the allergic lung disease phenotype was analyzed as previously described (Kheradmand et al., 2002).

Quantitative PCR.

Quantitative PCR of miRNAs and mRNAs were performed by using Taqman miRNA expression and gene expression assays, respectively (Applied Biosystems, Foster City, Calif.). PCR data was analyzed by using delta delta Ct method of relative quantification. For microRNA expression, either snoRNA202 and RNU48 were used as endogenous controls and for gene expression, GAPDH was used as the endogenous control.

Statistical Analysis.

For all statistical analyses, ANOVA with post hoc Tukey tests or t-tests were used. Statistical significance were calculated with P-value <0.05.

Mice and Allergen Challenge.

Prior to the intranasal administration of allergens, female C57BL/6 mice between 4 and 7 weeks of age, were anesthetized in an airtight chamber purged with a 3.2% isoflurane in oxygen vapor mixture for 10 minutes to achieve deep general anesthesia. Anesthetized mice received intranasally 45 mL chicken ovalbumin (22.5 mg) and 9 mL of protease derived from Aspergillus melleus (formerly A. oryzae; Sigma chemical company, St. Louis, Mo.; 7 mg) in PBS by applying droplets to the nares with a pipette. Allergen challenged mice received intranasal allergen on a schedule of every alternate day for eight total challenges and lungs were removed 24 hours after the final challenge. Lungs were perfused with ice cold, sterile normal saline to remove blood and collected in Trizol (Invitrogen, Carlsbad, Calif.). Total RNA was extracted by chloroform-ethanol method.

Small RNA Mapping and Classification.

After filtering for the Illumina small RNA adapter sequences, the reads were mapped to the reference mouse genome (NCBI Build 37, UCSC mm9) using the Pash software package (Kalafus et al., 2004). Pash anchoring used contiguous seeds of size 11 and masked out 5% of the genome containing highly repetitive sequences. The mapping results were uploaded to Genboree (www.genboree.com) for visualization. Reads with mappings that overlap miRNAs (miRBase version 14.0), piRNAs (piRNABank—pirnabank.ibab.ac.in), snoRNAs (RNAdb—jsm-research.imb.uq.edu.au/rnadb), genes (UCSC Genes track genome.ucsc.edu), or repeats (UCSC Repeat Masker track—genome.ucsc.edu) were identified. In the case of repeats each mapping is associated with specific types of repeat such as LINES, SINES, DNA or RNA.

Novel miRNA Discovery.

All small RNA sequences that failed to align with a known miRNA, piRNA or snoRNA were passed through a novel miRNA discovery platform. Each sequence was first mapped on the reference genome sequence (mm9) and 100 bases of flanking the sequence on either side were extracted to find the putative hairpin. The extracted sequence was then folded using the Vienna RNA folding package (Zuker and Jacobson, 1998). This provides the secondary RNA structure and associated minimum free energy (mfe) structure of each occurrence of the original small RNA sequence on the reference genome. To determine if a structure forms a plausible miRNA hairpin, a multi-stage “folding filter” was applied. This folding filter enforces three minimally restrictive miRNA hairpin rules: 1) The putative miRNA sequence must rest on one side of a single hairpin. Any more complicated structure involving the miRNA sequence is rejected since the definition of a miRNA requires that it form a simple, single-hairpin precursor. 2) The putative miRNA sequence must bind relatively tightly within the hairpin. Since miRNA biogenesis dictates that the precursor will be edited down to a short double stranded RNA involving the miRNA, it is understood that the miRNA sequence must bind relatively tightly within the hairpin. Following this, the inventors edited the folded MCE-MIR plus flanking sequence down to just the subsequence involved in the hairpin structure itself. It is important to edit and refold to ensure that the hairpin predicted is stable on its own, and not artificially stabilized by nearby structural elements. After editing and refolding the inventors checked to see if the refolded sequence met the final rule, 3) the putative hairpin must have a miRNA-appropriate energy (free energy below −20 kcal/mol). A small RNA sequence was identified as a putative novel miRNA if all these criteria were met.

Plasmids and Locked Nucleic Acids (LNA).

LNAs were purchased from Exiqon (Woburn, Mass.). For in vitro transfection, full-length LNAs anti-complementary to let-7a (5′-AACTATACAACCTACTACCTCA-3′ (SEQ ID NO:246)) and let-7e (5′-AACTATACAACCTCCTACCTCA-3′ (SEQ ID NO:247)) were used together with control LNAs anti-mmu-mir-705 (5′-TGCCCACCCCACCTCCCAC-3′ (SEQ ID NO:282)) and scrambled LNA (5′-AGAGCTCCCTTCAATCCAAA-3′ (SEQ ID NO:283)). For in vivo transfections, a truncated anti-let-7a,b,c.d LNA (5′-CAACCTACTACCTC-3′ (SEQ ID NO:248)) was used together with scrambled LNA (5′-AGAGCTCCCTTCAAT-3′ (SEQ ID NO:284)). MicroRNA and 3′UTR expression clones (let-7a: MmiR3368-MR01; mmu-mir-705: MmIR3181-MR01; scrambled miRNA: CmIR001-MR01; IL13: MmiT027416-MT01; scrambled 3′UTR: CmiT000001-MT01 were purchased from Genecopoeia (Rockville, Md.).

Isolation, Culture, and Transfection of CD4+ T Cells from Spleen.

Mouse spleens were collected and CD4 T cells isolated by immunomagnetic selection (Miltenyi). Briefly, suspensions of splenocytes were prepared by lightly pushing spleens through 40-um nylon strainers (BD Bioscinces, Durham, N.C.). CD4+ T lymphocytes were purified by using CD4 (L3T4) MicroBeads (Milteny Biotech, Auburn, Calif.) according to the manufacturer's protocol. For Th1 and Th2 differentiation, CD4+ T cells were added to 96-well plates that were coated with anti-CD3 (1 ng/mL) antibodies. While all the cells received anti-CD28 (1-μg/mL) and IL-2 (50 U/mL) antibodies, for Th1 differentiation, IL-12 (40-ng/mL) and anti-IL-4 (10-μg/mL) were added, whereas, for Th2 differentiation, IL-4 (20-ng/mL), anti-IFN-γ (10-μg/mL) and anti-IL-12 (10-μg/mL) were added and the cells were cultured for two weeks.

Nucleofection of in vitro anti-let-7a LNAs in to CD4 T cells was performed by using mouse T cell nucleofector kit (Lonza, Walkersville, Md.) according to the manufacturer's protocol and the cells were cultured for 48 hours. 80 and 240 pmol of anti-let-7a LNAs and 240 pmol of scrambled LNA were used for transfection. For determining the efficiency of transfection, cells that were transfected with flourescein labeled LNAs were nucleofected in to CD4 T cells and subjected to flow cytometry after 48 hours.

For RNA extraction, cells were homogenized in Trizol and total RNA was isolated using miRNeasy kit (Qiagen, Valencia, Calif.) according to the manufacturer's protocol.

Quantitation of Allergic Lung Disease.

24 hours after the final allergen challenge, the allergic lung disease phenotype was analyzed as previously described (Kheradmand et al., 2002). Briefly, mice were anesthetized with etomidate and placed on a mechanical ventilator inside a custom-designed rodent plethysmograph. Airway hyperresponsiveness (AHR) was assessed by determining the change in respiratory system resistance (R_{RS}) induced by provocative challenge with graded intravenous acetylcholine (Ach; dose expressed as mg/g body weight) as described (Kheradmand et al., 2002). Bronchoalveolar lavage fluid (BALF) was collected by instilling and withdrawing 1.6 ml of sterile phosphate buffered saline (PBS) through the tracheal cannula in two aliquots of 0.8 ml. BALF total and differential cell counts were performed using a standard hemocytometer and H&E staining of cytospin slides as described (Kheradmand et al., 2002). Quantitation of cytokines from BAL fluid was performed by bead-assisted analysis (MILLIPLEX MAP Kit Mouse Cytokine/Chemokine Immunoassay; Millipore, Billerica, Mass., USA) using a Bioplex analyzer (BioRad, Hercules, Calif.) according to the manufacturers' protocols. CD4 T cells from spleen were isolated and total RNA was extracted as described for lung.

### Example 2

### Pro-Inflammatory Role for Let-7 MicroRNAs in Experimental Asthma

miRNAs Dominate the Lung Short RNAome and Several are Edited to Alter the Target Repertoire.

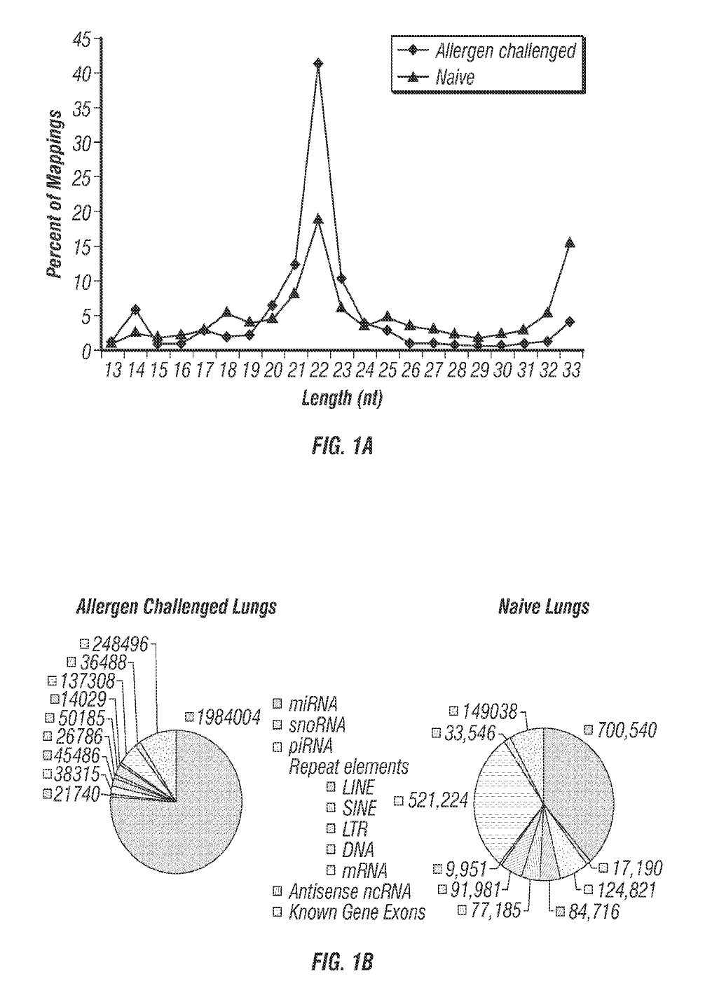

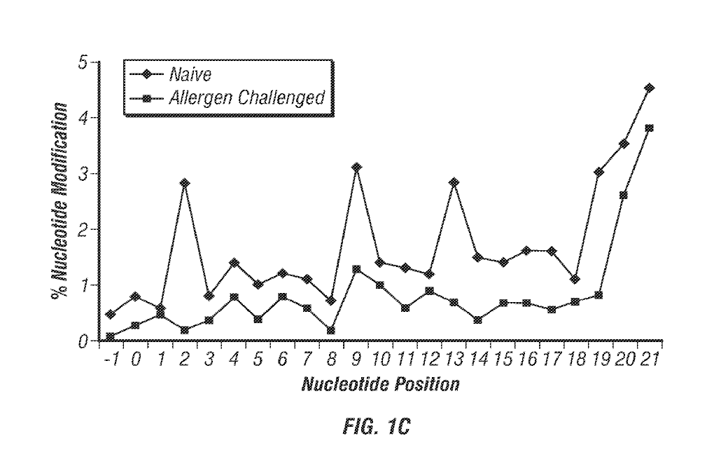

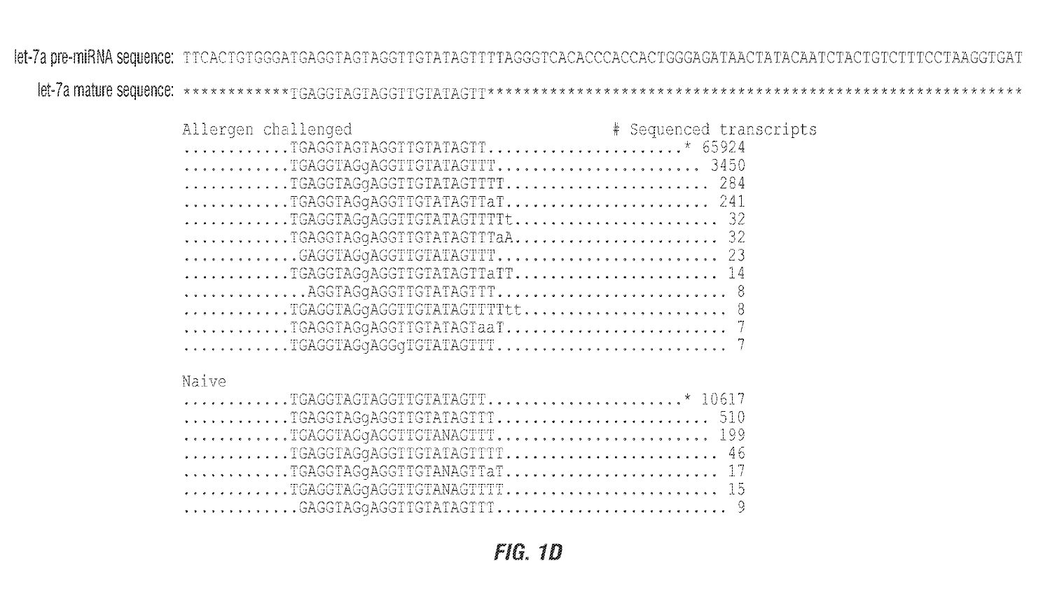

The lung short RNAomes of naïve and allergen challenged mice were characterized using the Genboree platform for mapping NGS data from the <60 nucleotide lung RNA fraction derived under each condition. This analysis revealed significant differences in the length distribution of small RNAs in naïve and allergen challenged lungs (FIG. 1A). The 21-23 nt RNA fraction was highly enriched in allergic as compared to naïve lung, whereas in the latter an increase in the 31-33 nt small RNA fraction was observed. miRNAs numerically dominated the short RNAome of both naïve and allergen-challenged animals, but numerous additional transcript classes were detected (FIG. 1B). Of particular interest were the Piwi-interacting RNAs (piRNAs) which previously were believed to be expressed only in haploid (gonadal) tissues of mammals (Xu et al., 2008). NGS identified a total of 405 distinct miRNAs (>10 copies of complete sequences identified each) in naïve and 328 miRNAs in allergen challenged mice (Tables 4 and 5). Let-7 family miRNAs were dominant, comprising 58% and 64% of total lung miRNAs from naïve and allergen challenged lungs, respectively (Tables 4 and 5). Among these, let-7f was most abundant in both naïve and allergen challenged lungs.

TABLE 4
Distribution of sequence reads aligning
with miRNAs in Naive Lung.

| | Naiive Lung | | |
|---|---|---|---|
| | Exact Match | | Match to |
| | to miRNA | Exact Match | miRNA with 1-3 |
| miRNA_miRBase 14.0 | (+/−4) | to miRNA | mismatches |
| mmu-let-7a | 31636 | 21224 | 11584 |
| mmu-let-7b | 25572 | 11208 | 26577 |
| mmu-let-7c | 75537 | 49150 | 28947 |
| mmu-let-7d | 10833 | 7107 | 2780 |
| mmu-let-7e | 2756 | 1726 | 816 |
| mmu-let-7f | 83754 | 61224 | 16859 |
| mmu-let-7g | 13090 | 10580 | 3197 |
| mmu-let-7i | 4550 | 3223 | 6453 |
| mmu-mir-1 | 27914 | 24720 | 3803 |
| mmu-mir-100 | 104 | 52 | 38 |
| mmu-mir-101a | 5412 | 747 | 3492 |
| mmu-mir-101b | 1647 | 168 | 565 |
| mmu-mir-103 | 5301 | 2960 | 2806 |
| mmu-mir-106a | 10 | 10 | 31 |
| mmu-mir-106b | 198 | 85 | 46 |
| mmu-mir-107 | 1103 | 305 | 62 |
| mmu-mir-10a | 3341 | 406 | 1118 |
| mmu-mir-10b | 16 | 0 | 0 |
| mmu-mir-1195 | 0 | 0 | 5 |
| mmu-mir-1197 | 0 | 0 | 74 |
| mmu-mir-125a-3p | 5 | 0 | 0 |
| mmu-mir-125a-5p | 102 | 5 | 7 |
| mmu-mir-125b-5p | 326 | 212 | 86 |
| mmu-mir-126-3p | 1114 | 404 | 0 |
| mmu-mir-126-5p | 245 | 206 | 295 |
| mmu-mir-127 | 54 | 43 | 504 |
| mmu-mir-1274a | 0 | 0 | 1598 |
| mmu-mir-128 | 696 | 336 | 207 |
| mmu-mir-129-5p | 0 | 0 | 5 |
| mmu-mir-130a | 520 | 490 | 280 |
| mmu-mir-130b | 73 | 73 | 20 |
| mmu-mir-132 | 5 | 5 | 13 |
| mmu-mir-133a | 130 | 0 | 20 |
| mmu-mir-133b | 2 | 0 | 0 |
| mmu-mir-136 | 0 | 0 | 200 |
| mmu-mir-138 | 0 | 0 | 1365 |
| mmu-mir-139-5p | 10 | 0 | 49 |
| mmu-mir-140 | 9 | 9 | 6 |
| mmu-mir-141 | 20 | 13 | 0 |
| mmu-mir-142-3p | 234 | 9 | 0 |
| mmu-mir-142-5p | 1507 | 417 | 278 |
| mmu-mir-143 | 11123 | 4546 | 8044 |
| mmu-mir-145 | 5739 | 2901 | 2970 |
| mmu-mir-146a | 733 | 298 | 700 |
| mmu-mir-146b | 472 | 80 | 290 |
| mmu-mir-148a | 266 | 201 | 142 |
| mmu-mir-148b | 160 | 138 | 105 |
| mmu-mir-149 | 0 | 0 | 30 |
| mmu-mir-150 | 71 | 34 | 10 |
| mmu-mir-151-3p | 81 | 25 | 0 |
| mmu-mir-151-5p | 306 | 146 | 37 |
| mmu-mir-152 | 1030 | 918 | 1145 |
| mmu-mir-155 | 13 | 6 | 0 |
| mmu-mir-15a | 210 | 44 | 156 |
| mmu-mir-15b | 491 | 244 | 23 |
| mmu-mir-16 | 1784 | 1096 | 461 |
| mmu-mir-17 | 94 | 65 | 32 |
| mmu-mir-181a | 6060 | 1436 | 1762 |
| mmu-mir-181b | 1422 | 196 | 816 |
| mmu-mir-181c | 65 | 13 | 28 |
| mmu-mir-181d | 406 | 129 | 216 |
| mmu-mir-182 | 12 | 7 | 22 |
| mmu-mir-1839-3p | 6 | 0 | 0 |
| mmu-mir-1839-5p | 952 | 466 | 626 |
| mmu-mir-185 | 680 | 567 | 234 |
| mmu-mir-186 | 50 | 15 | 0 |
| mmu-mir-187 | 7 | 7 | 29 |
| mmu-mir-1899 | 0 | 0 | 2859 |
| mmu-mir-18a | 12 | 0 | 0 |
| mmu-mir-1903 | 0 | 0 | 5 |
| mmu-mir-191 | 2225 | 662 | 747 |
| mmu-mir-192 | 549 | 232 | 188 |
| mmu-mir-193 | 204 | 168 | 56 |
| mmu-mir-1930 | 0 | 0 | 14 |
| mmu-mir-1934 | 0 | 0 | 14 |
| mmu-mir-1937a | 478 | 36 | 491 |
| mmu-mir-1937b | 478 | 134 | 0 |
| mmu-mir-1938 | 0 | 0 | 9 |
| mmu-mir-1939 | 0 | 0 | 802 |
| mmu-mir-194 | 14 | 0 | 0 |
| mmu-mir-1940 | 12 | 7 | 0 |
| mmu-mir-1944 | 6 | 6 | 0 |
| mmu-mir-1947 | 0 | 0 | 54 |
| mmu-mir-195 | 418 | 46 | 180 |
| mmu-mir-1950 | 0 | 0 | 353 |
| mmu-mir-1955 | 0 | 0 | 22 |
| mmu-mir-1956 | 0 | 0 | 73 |
| mmu-mir-1957 | 58 | 0 | 876 |
| mmu-mir-1959 | 1148 | 0 | 4505 |
| mmu-mir-1961 | 59 | 0 | 23 |
| mmu-mir-1965 | 0 | 0 | 17 |
| mmu-mir-1967 | 0 | 0 | 6 |
| mmu-mir-1968 | 0 | 0 | 177 |
| mmu-mir-196a | 0 | 0 | 10 |
| mmu-mir-1971 | 0 | 0 | 10 |
| mmu-mir-1983 | 0 | 0 | 99 |
| mmu-mir-199a-3p | 5820 | 2082 | 0 |
| mmu-mir-199a-5p | 38 | 4 | 0 |
| mmu-mir-199b | 5820 | 2082 | 0 |
| mmu-mir-19b | 64 | 24 | 6 |
| mmu-mir-200a | 593 | 169 | 210 |
| mmu-mir-200b | 291 | 135 | 131 |
| mmu-mir-200c | 326 | 146 | 86 |
| mmu-mir-201 | 0 | 0 | 23 |
| mmu-mir-203 | 85 | 33 | 60 |
| mmu-mir-205 | 57 | 17 | 5 |
| mmu-mir-206 | 13 | 13 | 0 |
| mmu-mir-208a | 14 | 14 | 17 |
| mmu-mir-20a | 42 | 30 | 17 |
| mmu-mir-20b | 20 | 12 | 0 |
| mmu-mir-21 | 5169 | 1521 | 902 |
| mmu-mir-210 | 21 | 16 | 6 |
| mmu-mir-2135 | 70 | 0 | 7 |
| mmu-mir-2137 | 0 | 0 | 10 |
| mmu-mir-2138 | 1342 | 0 | 0 |
| mmu-mir-214 | 30 | 19 | 38 |
| mmu-mir-2140 | 649 | 0 | 0 |
| mmu-mir-2141 | 0 | 0 | 428 |
| mmu-mir-2142 | 7892 | 30 | 1514 |
| mmu-mir-2143 | 9 | 0 | 0 |
| mmu-mir-2144 | 49 | 0 | 0 |
| mmu-mir-2145 | 1959 | 110 | 59 |
| mmu-mir-2146 | 777 | 0 | 221 |
| mmu-mir-215 | 5 | 0 | 19 |
| mmu-mir-218 | 8 | 8 | 0 |
| mmu-mir-2182 | 0 | 0 | 5 |
| mmu-mir-219 | 25 | 6 | 0 |
| mmu-mir-22 | 1292 | 966 | 236 |
| mmu-mir-221 | 781 | 142 | 470 |
| mmu-mir-222 | 274 | 90 | 84 |
| mmu-mir-223 | 44 | 17 | 45 |
| mmu-mir-224 | 14 | 0 | 25 |
| mmu-mir-23a | 3567 | 517 | 1845 |
| mmu-mir-23b | 1501 | 265 | 1293 |
| mmu-mir-24 | 2666 | 1402 | 6994 |
| mmu-mir-25 | 812 | 601 | 321 |
| mmu-mir-26a | 5440 | 4044 | 4841 |
| mmu-mir-26b | 2097 | 263 | 923 |
| mmu-mir-27a | 1490 | 287 | 769 |
| mmu-mir-27b | 912 | 319 | 929 |
| mmu-mir-28 | 38 | 11 | 0 |
| mmu-mir-293 | 0 | 0 | 6 |
| mmu-mir-296-5p | 9 | 9 | 0 |
| mmu-mir-298 | 13 | 0 | 5 |
| mmu-mir-29a | 11214 | 8326 | 3098 |
| mmu-mir-29b | 524 | 344 | 60 |
| mmu-mir-29c | 1106 | 812 | 229 |
| mmu-mir-302a | 0 | 0 | 11 |
| mmu-mir-30a | 4981 | 953 | 2831 |
| mmu-mir-30b | 260 | 227 | 34 |
| mmu-mir-30c | 498 | 90 | 124 |
| mmu-mir-30d | 1813 | 284 | 857 |
| mmu-mir-30e | 327 | 29 | 353 |
| mmu-mir-31 | 186 | 42 | 42 |
| mmu-mir-32 | 5 | 0 | 0 |
| mmu-mir-320 | 1430 | 849 | 1547 |
| mmu-mir-322 | 379 | 73 | 98 |
| mmu-mir-323-5p | 0 | 0 | 19 |
| mmu-mir-324-5p | 22 | 22 | 0 |
| mmu-mir-326 | 6 | 6 | 0 |
| mmu-mir-33 | 196 | 139 | 70 |
| mmu-mir-331-3p | 86 | 49 | 0 |
| mmu-mir-331-5p | 0 | 0 | 58 |
| mmu-mir-335-5p | 82 | 23 | 13 |
| mmu-mir-338-3p | 6 | 0 | 0 |
| mmu-mir-339-5p | 22 | 8 | 5 |
| mmu-mir-340-5p | 143 | 109 | 7 |
| mmu-mir-341 | 0 | 0 | 16 |
| mmu-mir-342-3p | 212 | 120 | 0 |
| mmu-mir-345-3p | 29 | 0 | 0 |
| mmu-mir-345-5p | 14 | 0 | 0 |
| mmu-mir-34a | 29 | 16 | 11 |
| mmu-mir-34b-3p | 46 | 21 | 0 |
| mmu-mir-34b-5p | 385 | 228 | 11 |
| mmu-mir-34c | 8789 | 3831 | 3359 |
| mmu-mir-350 | 53 | 16 | 0 |
| mmu-mir-351 | 12 | 0 | 0 |
| mmu-mir-361 | 20 | 14 | 47 |
| mmu-mir-362-3p | 26 | 13 | 0 |
| mmu-mir-362-5p | 7 | 0 | 0 |
| mmu-mir-363 | 0 | 0 | 12 |
| mmu-mir-365 | 28 | 28 | 0 |
| mmu-mir-370 | 0 | 0 | 10 |
| mmu-mir-374 | 104 | 83 | 6 |
| mmu-mir-375 | 307 | 259 | 69 |
| mmu-mir-376a | 8 | 8 | 0 |
| mmu-mir-378 | 1462 | 588 | 1316 |
| mmu-mir-379 | 12 | 12 | 11 |
| mmu-mir-382 | 0 | 0 | 7 |
| mmu-mir-411 | 15 | 7 | 0 |
| mmu-mir-423-3p | 39 | 30 | 0 |
| mmu-mir-423-5p | 895 | 577 | 174 |
| mmu-mir-425 | 20 | 12 | 6 |
| mmu-mir-429 | 111 | 50 | 27 |
| mmu-mir-448 | 0 | 0 | 35 |
| mmu-mir-449a | 205 | 176 | 26 |
| mmu-mir-449c | 30 | 0 | 0 |
| mmu-mir-450a-5p | 20 | 20 | 0 |
| mmu-mir-450b-3p | 10 | 10 | 0 |
| mmu-mir-451 | 89 | 28 | 14 |
| mmu-mir-453 | 0 | 0 | 49 |
| mmu-mir-455 | 41 | 9 | 8 |
| mmu-mir-466a-3p | 5 | 0 | 0 |
| mmu-mir-466b-3-3p | 1 | 0 | 0 |
| mmu-mir-466b-3p | 5 | 0 | 0 |
| mmu-mir-466c-3p | 5 | 0 | 0 |
| mmu-mir-466e-3p | 5 | 0 | 0 |
| mmu-mir-467a | 24 | 18 | 0 |
| mmu-mir-467b | 24 | 0 | 0 |
| mmu-mir-467c | 0 | 0 | 7 |
| mmu-mir-467e | 12 | 12 | 0 |
| mmu-mir-470 | 0 | 0 | 13 |
| mmu-mir-471 | 0 | 0 | 4928 |
| mmu-mir-484 | 23 | 23 | 0 |
| mmu-mir-485 | 0 | 0 | 120 |
| mmu-mir-486 | 22 | 11 | 23 |
| mmu-mir-490 | 7 | 0 | 0 |
| mmu-mir-491 | 0 | 0 | 11 |
| mmu-mir-494 | 0 | 0 | 36 |
| mmu-mir-497 | 207 | 53 | 46 |
| mmu-mir-499 | 0 | 0 | 38 |
| mmu-mir-500 | 12 | 7 | 6 |
| mmu-mir-501-3p | 6 | 6 | 0 |
| mmu-mir-503 | 189 | 158 | 0 |
| mmu-mir-504 | 0 | 0 | 9 |
| mmu-mir-532-3p | 27 | 18 | 0 |
| mmu-mir-532-5p | 106 | 91 | 0 |
| mmu-mir-541 | 8 | 0 | 0 |
| mmu-mir-542-3p | 24 | 8 | 0 |
| mmu-mir-542-5p | 6 | 6 | 0 |
| mmu-mir-546 | 0 | 0 | 14 |
| mmu-mir-574-3p | 49 | 25 | 0 |
| mmu-mir-582-5p | 5 | 0 | 0 |
| mmu-mir-592 | 0 | 0 | 30 |
| mmu-mir-598 | 27 | 8 | 0 |
| mmu-mir-615-5p | 0 | 0 | 247 |
| mmu-mir-652 | 96 | 50 | 668 |
| mmu-mir-654-5p | 0 | 0 | 22 |
| mmu-mir-665 | 0 | 0 | 275 |
| mmu-mir-668 | 7 | 0 | 210 |
| mmu-mir-669a | 15 | 6 | 0 |
| mmu-mir-669c | 54 | 18 | 0 |
| mmu-mir-669h-5p | 0 | 0 | 35 |
| mmu-mir-672 | 31 | 31 | 0 |
| mmu-mir-674 | 111 | 25 | 91 |
| mmu-mir-676 | 33 | 7 | 5 |
| mmu-mir-685 | 0 | 0 | 6 |
| mmu-mir-690 | 290 | 26 | 55 |
| mmu-mir-695 | 0 | 0 | 5 |
| mmu-mir-697 | 0 | 0 | 10 |
| mmu-mir-700 | 0 | 0 | 20 |
| mmu-mir-703 | 0 | 0 | 9 |
| mmu-mir-707 | 0 | 0 | 6 |
| mmu-mir-708 | 13 | 6 | 0 |
| mmu-mir-709 | 12 | 0 | 19 |
| mmu-mir-715 | 0 | 0 | 27 |
| mmu-mir-717 | 0 | 0 | 9 |
| mmu-mir-718 | 0 | 0 | 9 |
| mmu-mir-720 | 47 | 26 | 206 |
| mmu-mir-744 | 425 | 329 | 439 |
| mmu-mir-760 | 0 | 0 | 7 |
| mmu-mir-763 | 0 | 0 | 10 |
| mmu-mir-7a | 46 | 8 | 0 |
| mmu-mir-805 | 645 | 36 | 70 |
| mmu-mir-871 | 0 | 0 | 6 |
| mmu-mir-872 | 90 | 24 | 34 |
| mmu-mir-876-5p | 0 | 0 | 5 |
| mmu-mir-879 | 9 | 0 | 0 |
| mmu-mir-92a | 330 | 14 | 267 |
| mmu-mir-92b | 178 | 25 | 170 |
| mmu-mir-93 | 323 | 204 | 109 |
| mmu-mir-96 | 7 | 7 | 11 |
| mmu-mir-98 | 96 | 74 | 27 |
| mmu-mir-99a | 561 | 234 | 345 |
| mmu-mir-99b | 622 | 400 | 468 |

TABLE 5
Distribution of sequence reads aligning
with miRNAs in Allergen Challenged Lung.

| | Allergen Challenged Lung | | |
|---|---|---|---|
| | Exact Match | | Match to |
| | to miRNA | Exact Match | miRNA with 1-3 |
| miRNA_miRBase 14.0 | (+/−4) | to miRNA | mismatches |
| mmu-let-7a | 182234 | 131808 | 38610 |
| mmu-let-7b | 117273 | 53199 | 85788 |
| mmu-let-7c | 289933 | 197288 | 68360 |
| mmu-let-7d | 37016 | 26666 | 6792 |
| mmu-let-7e | 17466 | 11810 | 4586 |
| mmu-let-7f | 279496 | 215576 | 33450 |
| mmu-let-7g | 65409 | 52382 | 11034 |
| mmu-let-7i | 39647 | 29829 | 36366 |
| mmu-mir-1 | 22694 | 20666 | 1435 |
| mmu-mir-100 | 284 | 165 | 108 |
| mmu-mir-101a | 8739 | 1254 | 3475 |
| mmu-mir-101b | 3571 | 304 | 1117 |
| mmu-mir-103 | 26674 | 16416 | 9071 |
| mmu-mir-106b | 120 | 44 | 40 |
| mmu-mir-107 | 4218 | 1215 | 269 |
| mmu-mir-10a | 10134 | 1442 | 1714 |
| mmu-mir-10b | 38 | 0 | 0 |
| mmu-mir-1198 | 22 | 15 | 14 |
| mmu-mir-1199 | 0 | 0 | 11 |
| mmu-mir-122 | 169 | 67 | 37 |
| mmu-mir-125a-3p | 9 | 0 | 0 |
| mmu-mir-125a-5p | 1159 | 89 | 243 |
| mmu-mir-125b-3p | 11 | 5 | 0 |
| mmu-mir-125b-5p | 1477 | 964 | 512 |
| mmu-mir-126-3p | 1842 | 719 | 0 |
| mmu-mir-126-5p | 634 | 546 | 330 |
| mmu-mir-127 | 309 | 238 | 726 |
| mmu-mir-1274a | 0 | 0 | 671 |
| mmu-mir-128 | 316 | 194 | 73 |
| mmu-mir-1306 | 0 | 0 | 12 |
| mmu-mir-130a | 666 | 636 | 221 |
| mmu-mir-130b | 47 | 47 | 5 |
| mmu-mir-132 | 13 | 13 | 21 |
| mmu-mir-133a | 20 | 0 | 0 |
| mmu-mir-134 | 5 | 5 | 0 |
| mmu-mir-135b | 37 | 18 | 6 |
| mmu-mir-136 | 5 | 0 | 84 |
| mmu-mir-138 | 17 | 6 | 783 |
| mmu-mir-139-3p | 43 | 5 | 0 |
| mmu-mir-139-5p | 54 | 8 | 129 |
| mmu-mir-140 | 10 | 10 | 0 |
| mmu-mir-141 | 84 | 7 | 18 |
| mmu-mir-142-3p | 221 | 10 | 0 |
| mmu-mir-142-5p | 3935 | 476 | 813 |
| mmu-mir-143 | 28345 | 10993 | 8529 |
| mmu-mir-144 | 5 | 5 | 0 |
| mmu-mir-145 | 622 | 311 | 159 |
| mmu-mir-146a | 1623 | 679 | 1205 |
| mmu-mir-146b | 12481 | 2284 | 7623 |
| mmu-mir-147 | 73 | 73 | 46 |
| mmu-mir-148a | 1158 | 878 | 297 |
| mmu-mir-148b | 445 | 381 | 207 |
| mmu-mir-150 | 121 | 73 | 25 |
| mmu-mir-151-3p | 325 | 89 | 0 |
| mmu-mir-151-5p | 868 | 415 | 73 |
| mmu-mir-152 | 4141 | 3863 | 2432 |
| mmu-mir-154 | 15 | 15 | 0 |
| mmu-mir-155 | 52 | 11 | 0 |
| mmu-mir-15a | 146 | 62 | 41 |
| mmu-mir-15b | 384 | 121 | 14 |
| mmu-mir-16 | 1002 | 688 | 277 |
| mmu-mir-17 | 67 | 39 | 7 |
| mmu-mir-181a | 8536 | 1832 | 1929 |
| mmu-mir-181b | 2092 | 242 | 1253 |
| mmu-mir-181c | 262 | 29 | 7 |
| mmu-mir-181d | 810 | 254 | 380 |
| mmu-mir-182 | 135 | 24 | 54 |
| mmu-mir-183 | 26 | 9 | 5 |
| mmu-mir-1839-3p | 16 | 0 | 0 |
| mmu-mir-1839-5p | 5300 | 2581 | 2347 |
| mmu-mir-184 | 121 | 102 | 51 |
| mmu-mir-185 | 1874 | 1506 | 751 |
| mmu-mir-186 | 145 | 38 | 9 |
| mmu-mir-187 | 25 | 15 | 96 |
| mmu-mir-188-5p | 0 | 0 | 12 |
| mmu-mir-1892 | 0 | 0 | 5 |
| mmu-mir-1893 | 0 | 0 | 10 |
| mmu-mir-1899 | 0 | 0 | 155 |
| mmu-mir-18a | 6 | 0 | 0 |
| mmu-mir-1901 | 0 | 0 | 5 |
| mmu-mir-191 | 4431 | 1677 | 1528 |
| mmu-mir-192 | 1731 | 715 | 493 |
| mmu-mir-193 | 103 | 93 | 22 |
| mmu-mir-1930 | 0 | 0 | 23 |
| mmu-mir-1937a | 280 | 12 | 429 |
| mmu-mir-1937b | 280 | 184 | 0 |
| mmu-mir-1939 | 0 | 0 | 759 |
| mmu-mir-193b | 15 | 10 | 0 |
| mmu-mir-194 | 46 | 22 | 0 |
| mmu-mir-1940 | 0 | 0 | 5 |
| mmu-mir-195 | 306 | 21 | 135 |
| mmu-mir-1950 | 0 | 0 | 16 |
| mmu-mir-1955 | 0 | 0 | 16 |
| mmu-mir-1957 | 145 | 0 | 914 |
| mmu-mir-1959 | 1060 | 0 | 762 |
| mmu-mir-1961 | 31 | 0 | 6 |
| mmu-mir-1964 | 21 | 21 | 19 |
| mmu-mir-1971 | 0 | 0 | 48 |
| mmu-mir-199a-3p | 37851 | 15036 | 5 |
| mmu-mir-199a-5p | 140 | 16 | 14 |
| mmu-mir-199b | 37851 | 15036 | 0 |
| mmu-mir-19b | 42 | 16 | 7 |
| mmu-mir-200a | 2960 | 787 | 857 |
| mmu-mir-200b | 1625 | 758 | 661 |
| mmu-mir-200c | 1313 | 478 | 348 |
| mmu-mir-203 | 487 | 236 | 174 |
| mmu-mir-205 | 5 | 0 | 0 |
| mmu-mir-206 | 13 | 13 | 0 |
| mmu-mir-20a | 27 | 21 | 5 |
| mmu-mir-21 | 100689 | 36933 | 13096 |
| mmu-mir-210 | 43 | 35 | 0 |
| mmu-mir-2137 | 0 | 0 | 29 |
| mmu-mir-2138 | 18 | 0 | 21 |
| mmu-mir-214 | 57 | 29 | 154 |
| mmu-mir-2142 | 187 | 0 | 5 |
| mmu-mir-2143 | 66 | 0 | 0 |
| mmu-mir-2145 | 45 | 0 | 0 |
| mmu-mir-215 | 45 | 29 | 6 |
| mmu-mir-218 | 6 | 6 | 6 |
| mmu-mir-219 | 20 | 4 | 0 |
| mmu-mir-22 | 2014 | 1020 | 409 |
| mmu-mir-221 | 10783 | 2524 | 5125 |
| mmu-mir-222 | 1817 | 319 | 728 |
| mmu-mir-223 | 106 | 47 | 118 |
| mmu-mir-224 | 66 | 0 | 0 |
| mmu-mir-23a | 8912 | 451 | 3880 |
| mmu-mir-23b | 3751 | 298 | 2291 |
| mmu-mir-24 | 2304 | 1604 | 5714 |
| mmu-mir-25 | 5357 | 4348 | 1265 |
| mmu-mir-26a | 6472 | 5310 | 7339 |
| mmu-mir-26b | 4047 | 420 | 1193 |
| mmu-mir-27a | 4159 | 142 | 672 |
| mmu-mir-27b | 4614 | 1266 | 2122 |
| mmu-mir-28 | 221 | 50 | 6 |
| mmu-mir-296-3p | 10 | 0 | 0 |
| mmu-mir-298 | 142 | 0 | 88 |
| mmu-mir-299 | 10 | 5 | 0 |
| mmu-mir-29a | 28931 | 23179 | 3891 |
| mmu-mir-29b | 1093 | 798 | 94 |
| mmu-mir-29c | 903 | 806 | 107 |
| mmu-mir-301a | 8 | 0 | 0 |
| mmu-mir-30a | 32244 | 7522 | 9016 |
| mmu-mir-30b | 407 | 388 | 55 |
| mmu-mir-30c | 954 | 140 | 183 |
| mmu-mir-30d | 13360 | 1354 | 3750 |
| mmu-mir-30e | 1615 | 93 | 1100 |
| mmu-mir-31 | 375 | 105 | 100 |
| mmu-mir-32 | 13 | 0 | 5 |
| mmu-mir-449c | 124 | 0 | 0 |
| mmu-mir-450a-5p | 74 | 46 | 0 |
| mmu-mir-450b-3p | 66 | 50 | 0 |
| mmu-mir-451 | 167 | 39 | 0 |
| mmu-mir-452 | 5 | 0 | 0 |
| mmu-mir-453 | 0 | 0 | 6 |
| mmu-mir-455 | 74 | 16 | 0 |
| mmu-mir-466h | 0 | 0 | 5 |
| mmu-mir-467a | 12 | 12 | 0 |
| mmu-mir-467b | 12 | 0 | 0 |
| mmu-mir-467c | 7 | 0 | 0 |
| mmu-mir-467e | 7 | 7 | 0 |
| mmu-mir-471 | 0 | 0 | 171 |
| mmu-mir-484 | 42 | 35 | 6 |
| mmu-mir-485 | 9 | 0 | 91 |
| mmu-mir-486 | 59 | 44 | 21 |
| mmu-mir-490 | 10 | 0 | 6 |
| mmu-mir-491 | 0 | 0 | 13 |
| mmu-mir-494 | 0 | 0 | 530 |
| mmu-mir-495 | 0 | 0 | 5 |
| mmu-mir-497 | 529 | 171 | 106 |
| mmu-mir-500 | 22 | 15 | 5 |
| mmu-mir-501-3p | 161 | 52 | 0 |
| mmu-mir-503 | 1493 | 1243 | 103 |
| mmu-mir-504 | 0 | 0 | 22 |
| mmu-mir-532-3p | 8 | 8 | 0 |
| mmu-mir-532-5p | 822 | 736 | 146 |
| mmu-mir-541 | 196 | 21 | 61 |
| mmu-mir-542-3p | 213 | 86 | 0 |
| mmu-mir-542-5p | 18 | 18 | 0 |
| mmu-mir-543 | 10 | 0 | 0 |
| mmu-mir-574-3p | 49 | 44 | 0 |
| mmu-mir-574-5p | 38 | 6 | 14 |
| mmu-mir-582-3p | 17 | 0 | 0 |
| mmu-mir-582-5p | 11 | 5 | 0 |
| mmu-mir-598 | 109 | 27 | 13 |
| mmu-mir-615-5p | 0 | 0 | 223 |
| mmu-mir-652 | 293 | 47 | 479 |
| mmu-mir-665 | 6 | 0 | 20 |
| mmu-mir-669c | 271 | 138 | 19 |
| mmu-mir-669h-5p | 0 | 0 | 30 |
| mmu-mir-672 | 841 | 678 | 91 |
| mmu-mir-674 | 251 | 48 | 116 |
| mmu-mir-676 | 149 | 32 | 0 |
| mmu-mir-691 | 0 | 0 | 6 |
| mmu-mir-693-5p | 0 | 0 | 5 |
| mmu-mir-695 | 0 | 0 | 43 |
| mmu-mir-697 | 0 | 0 | 11 |
| mmu-mir-700 | 0 | 0 | 49 |
| mmu-mir-708 | 16 | 8 | 0 |
| mmu-mir-720 | 90 | 55 | 126 |
| mmu-mir-744 | 3138 | 2406 | 2165 |
| mmu-mir-760 | 0 | 0 | 8 |
| mmu-mir-762 | 0 | 0 | 18 |
| mmu-mir-7a | 320 | 72 | 36 |
| mmu-mir-805 | 1239 | 216 | 380 |
| mmu-mir-872 | 428 | 122 | 38 |
| mmu-mir-879 | 5 | 0 | 0 |
| mmu-mir-9 | 6 | 6 | 0 |
| mmu-mir-92a | 856 | 38 | 825 |
| mmu-mir-92b | 626 | 48 | 460 |
| mmu-mir-93 | 282 | 221 | 67 |
| mmu-mir-96 | 14 | 14 | 0 |
| mmu-mir-98 | 960 | 753 | 136 |
| mmu-mir-99a | 2572 | 1414 | 1137 |
| mmu-mir-99b | 4013 | 2562 | 2350 |

Upon mapping of sequences to the miRBase-14.0 pre-miRNA database, allowing for 1-4 mismatches in the aligned reads to a given pre-miRNA, several miRNAs were detected that were post-transcriptionally modified (edited) in at least one position of the seed sequence. The distribution of nucleotide changes in relation to position for all miRNAs in naive and allergen challenged lungs are shown in FIG. 1C. When the normalized numbers of nucleotide modifications were compared, the miRNA mmu-mir-101a showed a 10% increase in the number of 8th nucleotide modifications from C-to-U in naïve lungs as compared to allergen challenged. Using the TargetScan 5.1 algorithm, it was observed that the target repertoire of the modified mir-101a species had been re-directed to be identical to that of mmu-mir-144. Among several predicted changes in the target repertoire, this edit potentially enhances affinity for several allergy-related genes, including GATA and CD28 (Das, Chen, Yang, Cohn, Ray and Ray 2001; Keane-Myers, Gause, Linsley, Chen and Wills-Karp 1997). However, in keeping with prior observations from pancreatic tissue and mouse ovary (Reid et al., 2008), post-transcriptional modifications were particularly common in the let-7 family of microRNAs. The most common such modification was a U to G change at position 9 (let-7a(9U→G)), which was detected by comparison of let-7a sequences with pre-mmu-let-7a (FIG. 1D). This post-transcriptional modification effectively converts let-7a to let-7e, which largely shares the same targets (TargetScan 5.1). Thus, post-transcriptional editing of multiple lung miRNAs occurs, potentially altering the target repertoire for some miRNAs.

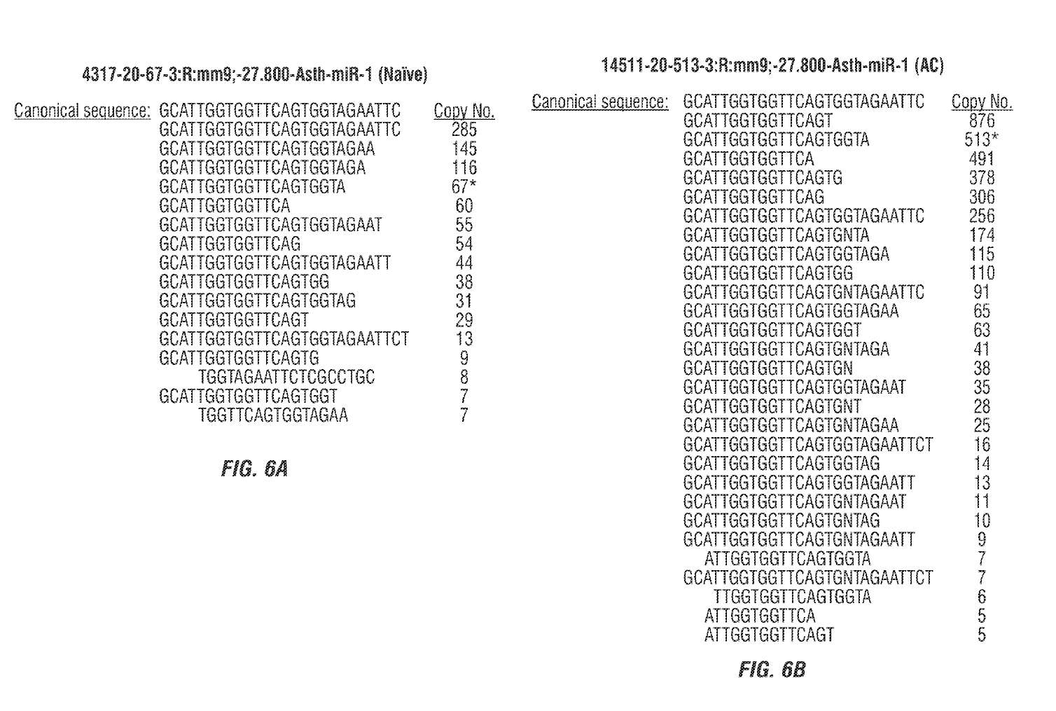

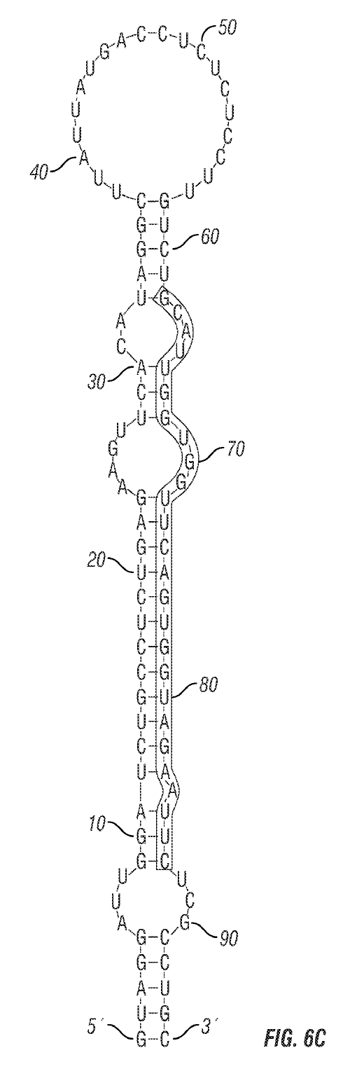

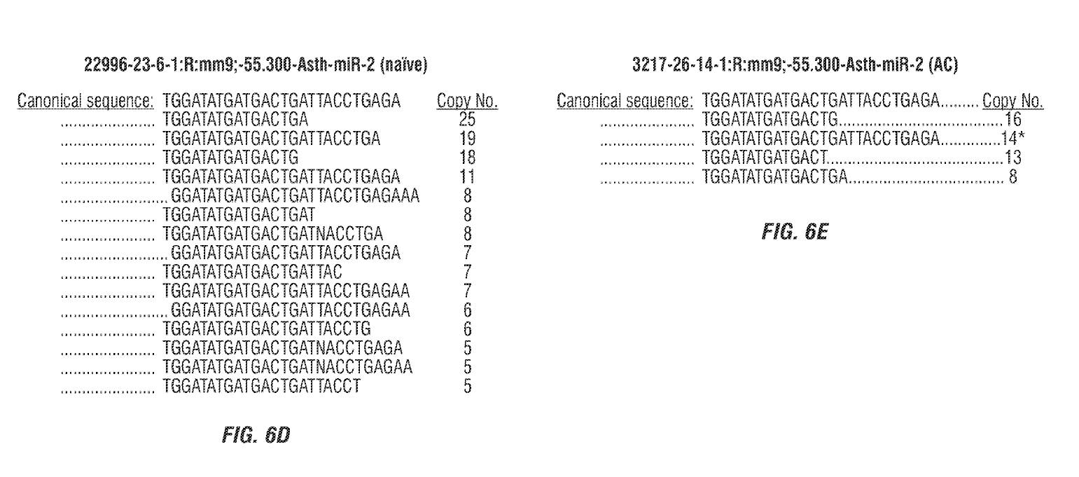

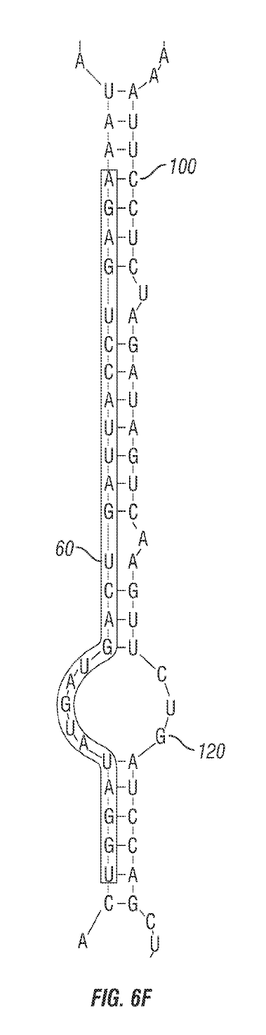

Using a novel miRNA discovery bioinformatics platform (Gu et al., 2008) the inventors further identified 25 putative novel miRNAs from naïve and allergen challenged lungs (Tables 6 and 7). Two miRNAs, Asth-miR-1 and Asth-miR-2, were highly expressed in naïve and possibly down regulated following allergen challenge (FIG. 6).

TABLE 6

| Putative | | | | | | | |
|---|---|---|---|---|---|---|---|
| Mature | SEQ ID | | | | Exact to | Exact to | Exact to |
| Mir(pmm) | NO: | Forward Sequence | Reverse Sequence | Hairpin | Hairpin | Plus 4 | pmm |
| 1 | 285 | TGAAGCGCGGGTA | | 8497-13-91- | 337 | 144 | 91 |
| | | | | 1:R:mm9;−35.100 | | | |
| 2 | 286 | GAAGGAACTACAAGACAGCT | | 12510-20-5- | 5 | 5 | 5 |
| | | | | 1:R:mm9;−27.900 | | | |
| 3 | 287 | CCCGGGTTTCGGCACCA | | 6746-17-208- | 1004 | 622 | 208 |
| | | | | 1:R:mm9;−97.000 | | | |
| 4 | 288 | TAACAGGTCTGTGA | | 9953-14-132- | 920 | 593 | 132 |
| | | | | 1:R:mm9;−29.900 | | | |
| 5 | 289 | AGATTGATTGTTAAGCTGAAA | GTTAGTGATGATCAATAAA | 5224-21-16- | 117 | 65 | 16 |
| | | | (SEQ ID NO: 290) | 1:R:mm9;−34.700 | | | |
| 6 | 291 | TGGGCTACACATTTT | | 13900-15-19- | 101 | 47 | 19 |
| | | | | 1:R:mm9;−33.300 | | | |
| 7 | 292 | AGCGATTTGTCTGG | | 2678-14-206- | 1466 | 1093 | 206 |
| | | | | 1:R:mm9;−26.100 | | | |
| 7.1 | 293 | AGCGATTTGTCTGG | | 2678-14-206- | 1245 | 1093 | 206 |
| | | | | 2:R:mm9;−28.300 | | | |
| 8 | 294 | GCATTGGTGGTTCAGT | | 9415-16-876- | 3274 | 2760 | 876 |
| | | | | 1:R:mm9;−27.800 | | | |
| 9 | 295 | CGCAGTTTTATCCGGTA | | 7178-17-39- | 327 | 84 | 39 |
| | | | | 1:R:mm9;−26.630 | | | |
| 9.1 | 296 | CGCAGTTTTATCCGGTA | | 7178-17-39- | 244 | 69 | 39 |
| | | | | 3:R:mm9;−28.300 | | | |
| 10 | 297 | GCGTTGGTGGTATAGTGGTGA | | 14359-21-372- | 1538 | 1538 | 372 |
| | | | | 1:R:mm9;−42.900 | | | |
| 11 | 298 | GGCTCCATAGCTCAGGG | | 10646-17-57- | 106 | 106 | 57 |
| | | | | 1:R:mm9;−27.600 | | | |
| 12 | 299 | GAGCACCCCATTGGCTACCCAC | | 13020-22-6- | 12 | 12 | 6 |
| | | | | 1:R:mm9;−77.240 | | | |
| 13 | 300 | GAAGATTAGCATGGCCCCTG | | 12450-20-13- | 74 | 29 | 13 |
| | | | | 1:R:mm9;−25.300 | | | |
| 14 | 301 | TGGATATGATGACTGA | | 14705-16-25- | 127 | 58 | 25 |
| | | | | 1:R:mm9;−55.300 | | | |
| 15 | 302 | GAAGGGCAAAAGCTCGCTTGATCTTGA | | 6423-27-14- | 57 | 52 | 14 |
| | | | | 1:R:mm9;−26.400 | | | |
| 16 | 303 | GTATGTGCTTGGCTGAGGA | | 15270-19-138- | 388 | 326 | 138 |
| | | | | 1:R:mm9;−33.000 | | | |
| 17 | 304 | CCCGGGTTTCGGCACCA | | 6746-17-208- | 1004 | 622 | 208 |
| | | | | 5:R:mm9;−98.500 | | | |
| 18 | 305 | ATCGTAATCTGAGCCGA | | 15481-16-28- | 299 | 50 | 28 |
| | | | | 1:R:mm9;−40.200 | | | |
| 18.1 | 306 | ATCGTAATCTGAGCCGA | | 5243-17-33- | 52 | 33 | 33 |
| | | | | 1:R:mm9;−35.400 | | | |
| 19 | 307 | TACCATGATCACGA | | 10320-14-14- | 59 | 14 | 14 |
| | | | | 1:R:mm9;−39.200 | | | |
| 20 | 308 | CTAAAATTGGAACGATACAGA | | 11096-21-10- | 45 | 35 | 10 |
| | | | | 1:R:mm9;−41.900 | | | |
| 21 | 309 | CGGAACTGAGGCCATGA | | 7495-17-68- | 519 | 389 | 68 |
| | | | | 1:R:mm9;−40.800 | | | |

TABLE 7

| | SEQ | | | | Exact | Exact | Exact |
|---|---|---|---|---|---|---|---|
| | ID | | | | to | to | to |
| | NO: | Forward Sequence | Reverse Sequence | Hairpin | Hairpin | Plus 4 | pmm |
| 1 | 310 | GCTAAGCAGGGTCGGGCCTGGTTA | GTCTACGGCCATACCACCCTGAA | 3470-24-25-1:R:mm9;−30.300 | 57 | 47 | 25 |
| | | | (SEQ ID NO: 311) | | | | |
| 2 | 312 | ACGGGAGGGCCGGGCGGCGAG | | 1267-21-5-1:R:mm9;−70.300 | 5 | 5 | 5 |
| 3 | 313 | GCATTGGTGGTTCAGTGGTAGAATTC | | 1764-26-285-1:R:mm9;−27.800 | 968 | 960 | 285 |
| 4* | 314 | GCGTTGGTGGTATAGTGGTGA | | 5553-21-19-1:R:mm9;−42.900 | 88 | 88 | 19 |
| 5 | 315 | GGTGGTGCAGGCAGGAGAGCCA | | 6579-22-5-1:R:mm9;−77.240 | 5 | 5 | 5 |
| 6 | 316 | TGGACACTGGAGAGAGAGCTTT | | 12241-22-7-1:R:mm9;−37.300 | 7 | 7 | 7 |
| 7 | 317 | GACTGCTGATCCGGGTGATGCGAA | | 3164-24-5-1:R:mm9;−39.300 | 5 | 5 | 5 |
| 8* | 318 | TGGATATGATGACTG | | 3675-15-16-1:R:mm9;−55.300 | 51 | 37 | 16 |
| 9 | 319 | GGGGGTATAGCTC | | 2283-13-62-1:R:mm9;−47.200 | 184 | 109 | 62 |
| 10* | 320 | CCCGGGTTTCGGCACCA | | 1641-17-34-1:R:mm9;−97.000 | 82 | 75 | 34 |
| 11 | 321 | GACGAGGTGGCCGAG | | 2115-15-19-1:R:mm9;−39.900 | 86 | 66 | 19 |
| 12 | 322 | TTGGGCAGAGGAGGCAGGGACA | | 13492-22-10-1:R:mm9;−32.100 | 10 | 10 | 10 |

*also found in naïve lung RNA

Identification of Relevant miRNA-mRNA Functional Pairs.

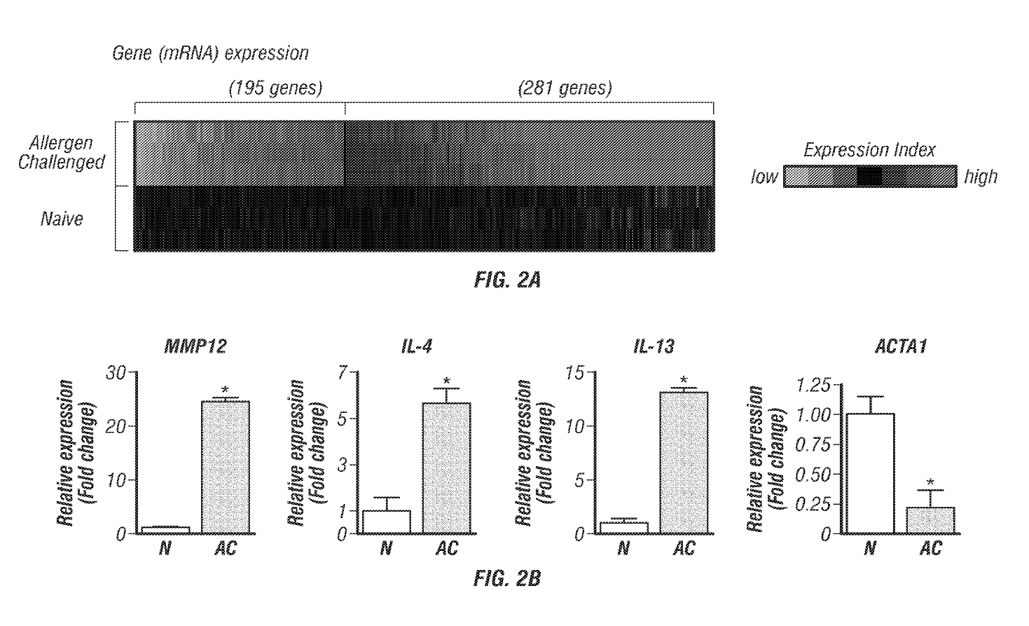

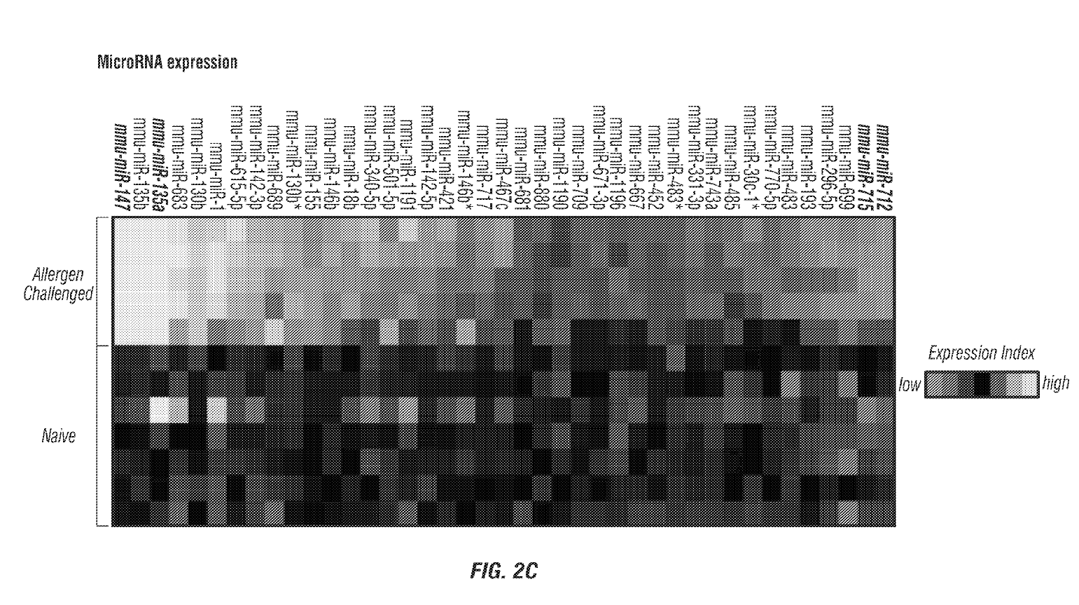

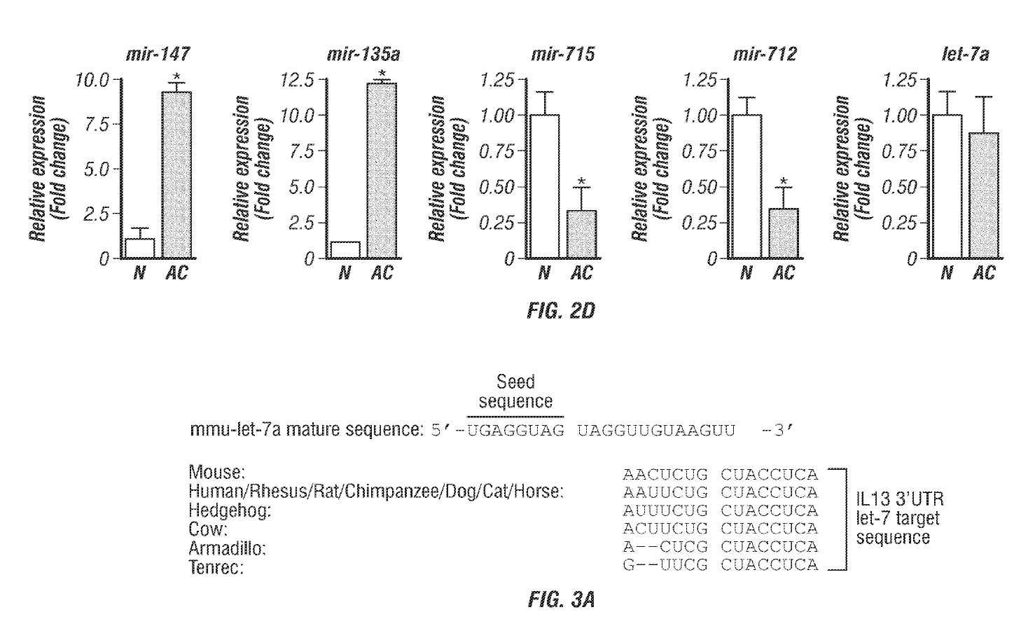

Deep sequencing is capable of identifying and enumerating both known and novel miRNAs as well as other classes of short transcripts, but the sensitivity of this technique for detecting and quantitating all known transcripts in complex samples such as lungs remains unknown. To circumvent this potential limitation of NGS, mRNA and miRNA microarray analyses were performed using total RNA from naive and allergen challenged mouse lungs and validated findings for selected genes using quantitative PCR (FIGS. 2A-D). A total of 195 genes were upregulated and 281 genes were downregulated in allergen challenged lungs relative to naïve (FIG. 2A). In addition to numerous immunoglobulin genes, the most highly induced genes included EAR11, an eosinophil-associated ribonuclease (Cormier et al., 2001), Gob-5 (CLCA3), a gene with uncertain function linked to allergic disease (Nakanishi et al., 2001), Ym2 (CHI3L4), a chitinase-like molecule that is induced by IL-4 (Webb et al., 2001), and matrix metalloproteinase 12 (MMP12), an IL-13-inducible proteinase that is required for allergen-induced airway eosinophilia (Pouladi et al., 2004). Enhanced expression of IL-4 and other Th2 cytokine transcripts was also detected in allergen-challenged lungs as expected, with the notable exception of IL-13.

Conversely, genes that were most prominently downregulated with allergen challenge included contractile proteins (alpha 1 actin (ACTA1); troponin C (TNNC2)), chemokines (CXCL14), ARNTL (BMAL1), a CLOCK-associated gene linked to glucose metabolism (Rudic et al., 2004), IFITM6 (fragilis5), and lysozyme. qRT-PCR analysis of selected genes validated mRNA transcripts that were either up- or downregulated (FIG. 2B). In contrast to microarray results, IL-13 transcripts were clearly markedly enhanced by allergen challenge as assessed by qRT-PCR (FIG. 2B). Moreover, the enhanced presence of both IL-13 transcript and protein in allergic lungs has been repeatedly documented (Arima et al. 2002; Corry et al., 1996; Grunig et al. 1998; Huang et al., 1995; Kasaian et al. 2007), indicating that the inability to detect this transcript by microarray was spurious. These studies thus confirm that numerous allergy-related genes are upregulated in lungs following allergen challenge.

Microarray analyses further identified numerous miRNAs that were significantly up- and down-regulated with allergen challenge (FIG. 2C). Expression of the most abundant miRNA transcripts, most notably let-7 miRNAs, did not change with allergen challenge. qRT-PCR again verified trends in expression of selected miRNAs that changed significantly and it was confirmed that let-7a transcripts were not altered by allergen challenge (FIG. 2D). Based on Targetscan 5.1 predictions, numerous miRNAs were identified from these analyses that putatively target genes of relevance to the asthma phenotype (Table 9). For example, a potential target of mir-135a, which was significantly up-regulated in asthmatic mice, is signal transducer and activator of transcription 6 (STATE), a transcription factor that is required for Th2 responses and experimental asthma (Kuperman et al., 1998).

TABLE 9
Lung miRNAs and potential targets
with relevance to allergic disease

| | Target | | Context |
|---|---|---|---|
| MicroRNA | gene | Gene name | Score* |
| mmu-mir-712 | GATA3 | GATA binding protein 3 | −0.12 |
| mmu-mir-699 | STAT6 | signal transducer and activator | −0.26 |
| | | of transcription 6 | |
| mmu-mir-743a | IL13RA1 | interleukin 13 receptor, alpha 1 | −0.4 |
| mmu-mir-1196 | GATA3 | GATA binding protein 3 | −0.33 |
| mmu-mir-709 | CD4 | CD4 | −0.23 |
| mmu-mir-717 | ADRB2 | adrenergic, beta-2-, receptor | −0.33 |
| mmu-mir-142-5p | JAK1 | Janus kinase 1 | −0.16 |
| mmu-mir-340-5p | IL4 | Interleukin 4 | −0.25 |
| mmu-mir-340-5p | JAK1 | Janus kinase 1 | −0.26 |
| mmu-mir-146b | IRAK1 | interleukin-1 receptor-associated | −0.91 |
| | | kinase 1 | |
| mmu-mir-135a | STAT6 | signal transducer and activator | −0.45 |
| | | of transcription 6 | |
| mmu-let-7 | IL13 | Interleukin 13 | — |

*Derived from TargetScan 5.1.

IL-13 is a Target Gene of let-7a.

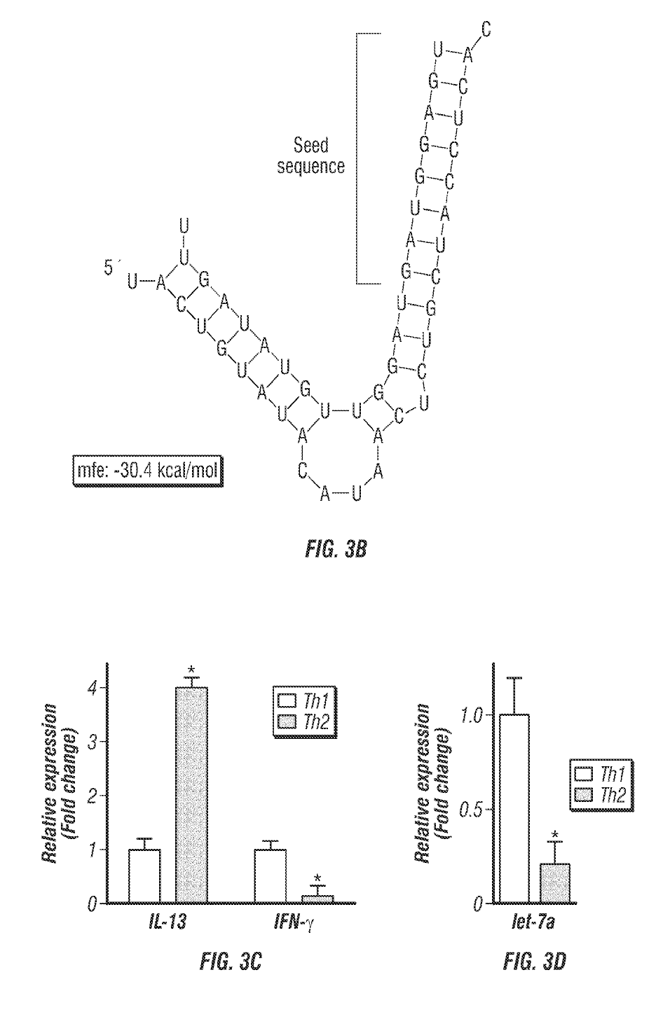

Subsequent efforts were focused on the abundant and extremely conserved let-7 miRNA family, the function of which in mammals remains largely undefined. The let-7 family target recognition sequence in the IL13 3′ UTR is highly conserved across mammalian species (FIG. 3A). Moreover, all mouse let-7 miRNAs (mmu-let-7a-i; mmu-mir-98) are predicted to target IL-13 (TargetScan 5.1). To verify this, the inventors first folded the mature let-7a-1 miRNA sequence against the mouse IL-13 3 ′UTR target sequence. This comparison revealed a high degree of complementarity characterized by a very low mean free energy value of −30.4 kcal/mol (FIG. 3B).

Lung IL-13 transcripts were markedly enhanced with allergen challenge (FIG. 2B) whereas total lung let-7a transcripts did not change (FIG. 2D), which failed to support a functional relationship between IL-13 and let-7a. However, Th2 cells are the predominant source of lung IL-13 following allergen challenge and represent a small (0.01-0.1%) fraction of total lung cells following allergen challenge in this model. Both IL-13 and mmu-let-7a transcripts in Th2 cells derived from naïve mouse CD4 T cells were quantitated. Similar to lung, let-7 miRNAs were the most abundant miRNA transcripts in T helper cells. As expected, IL-13 transcripts were markedly enhanced whereas interferon gamma (IFN-γ) transcripts were suppressed in Th2 relative to Th1 cells (FIG. 3C), but in contrast to lung, mmu-let-7a transcripts were markedly suppressed in Th2 cells, an inverse association with IL-13 that did suggest a functional interaction (FIG. 3D).

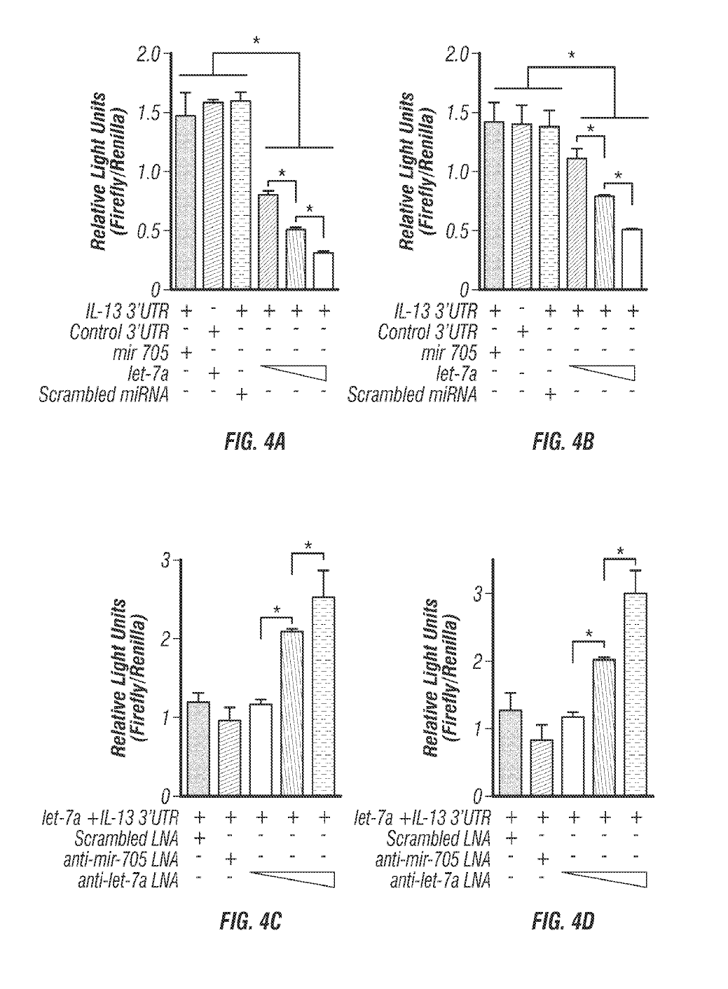

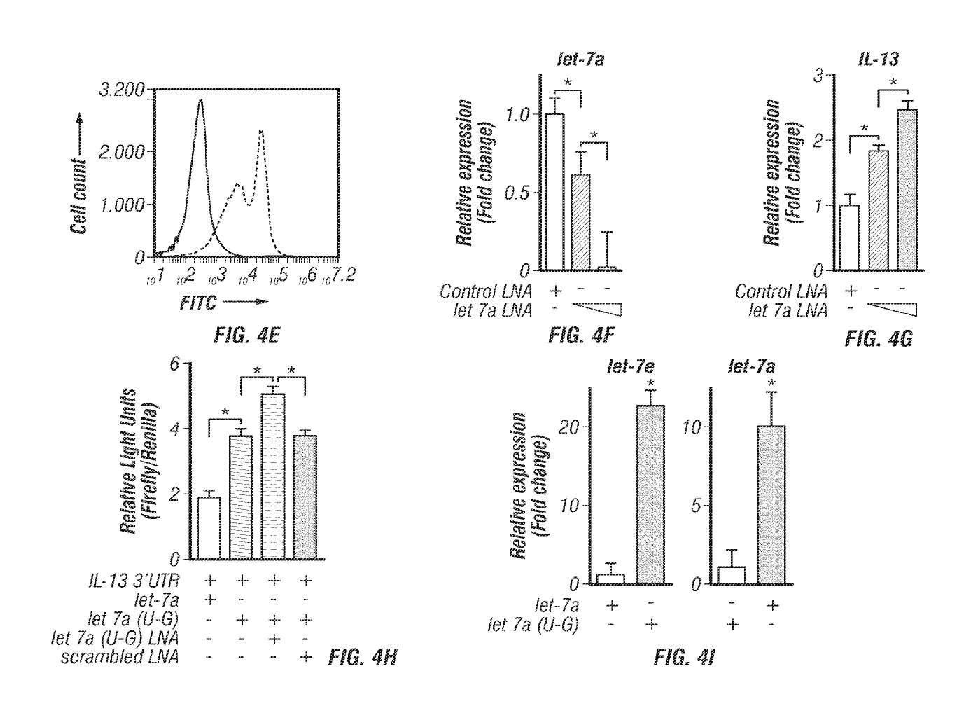

To determine if IL-13 is a genuine target of mmu-let-7a, plasmids expressing the pre-miRNA for mmu-let-7a and a luciferase gene containing the IL-13 3′UTR were co-transfected into HEK293T cells. In a dose-dependent manner, mmu-let-7a suppressed luciferase production, whereas neither a scrambled miRNA nor an irrelevant miRNA (mir-705) had any effect (FIG. 4A). Further, scrambled or anti-let-7a locked nucleic acids (LNA) (ref) representing the entire reverse complement of mmu-let-7a were transfected into these cells. Again in a dose dependent manner, anti-let-7a LNAs progressively reversed the suppressive effect of mmu-let-7a on luciferase production (FIG. 4B). Identical experiments were performed using the human IL-13 3′UTR, human let-7a (hsa-let-7a, which is identical to mmu-let-7a) and the same LNAs and produced identical results (FIGS. 4C, D). Together, these studies indicated that both human and mouse IL-13 are targets of let-7a and that this miRNA can be specifically inhibited by an LNA.

These findings were next confirmed in primary murine CD4+ T cells. The majority (>80%) of T helper cells could be transfected with anti-let-7a LNA (FIG. 4E), which by RT-qPCR reduced let-7a transcripts >90% at the highest LNA dose given (FIG. 4F). This was accompanied by a 2.5-fold greater increase in CD4 T cell IL-13 transcripts following activation (FIG. 4G). Together, these findings confirm that IL-13 is regulated by let-7a and demonstrate the utility of LNAs for the specific inhibition of miRNAs in primary T cells.

Finally, this in vitro system was used to compare native let-7a and let-7a(9U→G) for their ability to silence IL-13 expression. Despite having identical affinities for the IL-13 3′UTR recognition site (Targetscan 5.1), let-7a(9U→G) (let-7e) was less efficient in suppressing IL-13 expression relative to let-7a (FIG. 4H). The let-7a(9U→G) pre-miRNA as used in these studies is not identical to the let-7e pre-miRNA, raising the possibility that the let-7a(9U→G) pre-miRNA was not properly processed into mature let-7e. However in separate transfection experiments, it was confirmed by qRT-PCR that mature let-7e was fully processed from the let-7a(9U→G) pre-miRNA, as was mature let-7a from let-7a pre-miRNA (FIG. 41). Thus, editing of let-7a to let-7a(9U→G) creates let-7e, which is less efficient at suppressing IL-13 expression.

Pro-Inflammatory Role of Let-7 miRNAs In Vivo.

In addition to IL-13, let-7 miRNAs are predicted to inhibit other genes of interest in asthma, including the beta-2-adrenergic receptor (β₂-AR; ADRB2), a catecholamine receptor that is required for expression of experimental allergic lung disease (refs). However, the entire let-7 miRNA family is predicted to regulate over 800 conserved targets (TargetScan 5.1). It was reasoned that the overall in vivo function of mmu-let-7a, or indeed any miRNA, cannot alone be predicted from in silico analysis of the target repertoire even combined with knowledge of individually validated targets. Thus, to begin to assess overall function of let-7 miRNAs in vivo, allergen immunized mice were systemically administered either a scrambled or an anti-let-7 LNA that is the reverse complement of the first 14 nucleotides (5′) of let-7a, b, c and d. LNAs were administered before intranasal allergen challenge, but after allergen sensitization, to determine their effect on the effector phase of the disease (FIG. 5A). The specificity of this in vivo protocol was first evaluated, and it was observed that anti-let-7 LNA, but not a scrambled LNA, reduced let-7a transcripts in splenic CD4 T cells (FIG. 5B). However, unlike the immediate effect of anti-let-7a on T cells transfected in vitro (FIG. 4F, G), after 3 days of allergen challenge in vivo, splenic CD4 T cell IL-13 transcripts were reduced, whereas transcripts of an unrelated gene, IFN-γ, were unaffected (FIG. 5B).

The discrepancy in expression of the same target gene observed with immediate (FIGS. 4A-I) and delayed (FIGS. 5A-E) administration of an anti-let 7 LNA was unexpected and suggested that secondary or even tertiary effects of let-7 inhibition arise over time in vivo to suppress inflammatory gene expression. To determine if this anti-inflammatory effect is physiologically significant, the effect of anti-let-7 miRNAs on the allergic lung disease phenotype was determined. Two canonical features of this phenotype are airway hyperreactivity, which was determined in anesthetized, mechanically ventilated animals as the change in respiratory system resistance (R_{RS}) induced by graded injections of acetylcholine; and recruitment to the airways of inflammatory cells. As expected, scrambled LNA had no effect on these asthma-related parameters (FIG. 5C, D). In contrast, anti-let-7 LNA markedly suppressed both hyperresponsiveness to acetylcholine and lung inflammation, especially eosinophil recruitment to the airways. Analysis of airway cytokines confirmed that anti-let-7, but not control LNA significantly inhibited secretion of canonical Th2 cell cytokines including IL-4, IL-5 and IL-13 (FIG. 5E). In contrast, neither LNA influenced secretion of IFN-γ, ruling out a possible anti-viral response triggered by the exogenous LNAs. Thus, in contrast to expectations from analysis of individual gene targets in vitro, in vivo suppression of let-7 miRNAs revealed the pro-inflammatory role of select members of this miRNA family in allergic lung disease.

Using a combination of high-resolution miRNA microarrays and NGS together with detailed bioinformatic analyses, a whole genome view of major families of short transcripts and the RNAome of the lung in its naïve state and the changes it undergoes in response to challenge with a potent respiratory allergen are presented here. Lung miRNAs demonstrated profound changes in overall abundance, sequence, and composition of individual species. Many new miRNAs have been discovered through this effort and it was determined that let-7 microRNAs are the most abundant of all miRNAs in mouse lung. Although the majority of prior studies suggested a dominant anti-inflammatory role for miRNAs in immunity, in vivo analyses revealed a potent pro-inflammatory role for let-7 miRNAs in allergic lung disease. Together, these results constitute an important miRNA database and provide unique insight into the control of allergic inflammation.

Emerging evidence suggests that miRNA function is highly nuanced and can range from straightforward silencing to fine-tuning of gene expression (Reid et al., 2008). A striking finding of this study is that miRNA editing potentially represents a new dimension of this essential function. Previously, miRNAs of the let-7 family were observed to be extensively edited in cells derived from human and mouse pancreas and ovary and the current study extends this finding to the lung (Reid et al., 2008). It is shown here that relatively under-represented, non-let-7 miRNAs show similar editing. The C-to-U modification of mmu-mir-101a effectively converts the seed sequence to that of mmu-mir-144, with significant potential alterations in the target repertoire. Moreover, this data demonstrates that conversion of let-7a to let-7e (let-7a(9U→G)) reduces the ability of mmu-let-7a to regulate established targets such as IL-13. All let-7 miRNAs are predicted to target the same genes and let 7a- and let-7e appear to target IL-13 with identical affinity (TargetScan 5.1). These studies therefore indicate that subtleties exist with respect to the efficiency of target suppression relevant to position 9 nucleotides that are not accounted for by current prediction algorithms. Further analysis of the effect of miRNA edits, both naturally occurring and induced, on target regulation will be useful in refining the accuracy of target predictions.

Many of the novel miRNAs presented herein are homologous to transcripts previously identified from humans, zebra fish and mice as piRNAs. Some of the novel transcripts exceed the typical length of miRNAs (˜22 nt), e.g., Asth-miR-1 consists of 26 nt. However, the genomic context of all novel putative miRNAs permits the formation of a stable pre-miRNA duplex that may serve as a substrate for the nuclear Drosha/Pasha microprocessor required for miRNA biogenesis. Because piRNA precursors do not form such duplexes, and indeed the biogenesis of piRNAs remains uncertain (Kim et al., 2009), these novel sequences are most appropriately classified as miRNAs.

Identified herein are numerous miRNAs from mouse lung with potential relevance to the control of allergic inflammation as suggested by a limited analysis of the target repertoire. The data indicates a highly complex role played by miRNAs in this disease model. For the current study, additional effort ws focused on understanding the global significance of let-7 miRNAs to the control of allergic lung inflammation. This large miRNA family was chosen because of the high degree of conservation of family members across metazoans and unexpectedly robust expression in both T cells and lung that suggested a conserved and likely critical function (Lee and Ambros 2001). Let-7 miRNAs and the let-7 processing regulator Lin28 (Viswanathan et al., 2008) have previously been identified as regulators of developmental timing, morphogenesis and cancer (Hammell et al., 2009; Iliopoulos et al., 2009; Viswanathan et al. 2009). However, the miRNA-controlled cellular circuitry involved in development and oncongenesis overlaps with programs governing inflammation (Davidson-Moncada et al., 2010; Iliopoulos et al., 2009), suggesting that a regulatory role for let-7 miRNAs in lung inflammation was possible.

Although as predicted IL-13 is regulated by let-7a, given the more than 800 predicted targets of let-7 miRNAs, the inventors reasoned that the effects of let-7 inhibition in a complex in vivo model of inflammation could not be predicted based on target validation alone. Indeed, neither the failure of lung IL-13 and let-7a transcript expression to correlate inversely nor the suppressive effect of let-7a on T cell IL-13 transcripts predicted the requisite role of let-7 miRNAs in allergic lung disease. These findings emphasize the difficulty in predicting miRNA function in complex in vivo systems and indicate that the primary effects of let-7 inhibition on target gene expression translate over time into dominant secondary effects that ultimately suppress inflammation. The large size of the let-7 target repertoire and such secondary effects precluded precise identification of the pro-inflammatory mechanism coordinated by let-7 miRNAs, an effort made more complex by the recent discoveries that let-7 miRNAs can either promote or suppress target gene expression by binding either canonical or non-canonical mRNA elements (Lytle et al., 2007; Vasudevan, Tong and Steitz 2007).

Assessing the biological function of let-7 miRNAs in vivo is challenging. In addition to targeting essentially the same ˜820 mRNAs, the nine known let-7 miRNAs derive from 12 genetic loci (three exist as duplicate miRNA genes), effectively precluding a direct family-wide gene silencing approach through homologous recombination. For this study, LNAs were used since the safety, efficacy and specificity of which have been demonstrated both in vitro and in vivo (Elmen et al. 2008; Lanford et al., 2010; Wahlestedt et al. 2000). LNAs have the additional advantage over alternate gene silencing approaches that potentially toxic transfection vehicles (viruses, polyethyleneimine, etc.) are not required for in vivo use (Stein et al. 2010). The present studies confirm the specificity of LNAs used in vitro and in vivo and no toxicity was observed in mice receiving either control or anti-let-7 LNAs. These studies therefore support the therapeutic application of anti-let-7 LNAs in asthma and possibly other allergic conditions specifically to target let-7 and potentially numerous other miRNAs.

In summary, a variety of genomic approaches were used to demonstrate that numerous miRNAs and other short transcripts are expressed in mouse lung and undergo marked changes in abundance during the transition from the naïve state to allergic lung disease. Selected miRNAs undergo editing, creating potentially novel means for regulating the target repertoire and numerous novel miRNAs were identified. miRNAs of interest to allergic disease were identified, and it was demonstrated that the most abundant lung miRNAs, from the let-7 family, are required to support allergic lung disease.

### Example 3

### Effects of Inhibition of Let-7 miRNA-155 In Vivo

This Example describes the effects of the inhibition of mmu-mir-155 (mouse miRNA 155) in mice; as shown below, the data indicates that miRNA-155 is required for the expression of allergic lung responses in vivo. Novel miRNAs from mouse T cells were also identified.

Materials and Methods

Mice.

Four to six-week-old female Balb/c and C57BL/6 mice were obtained from The Jackson Laboratory (Bar Harbor, Me.). All mice were maintained at the Transgenic Mouse Facility (TMF) at Baylor College of Medicine (BCM) and treated in accordance with the institutional and federal guidelines of BCM and the National Institutes of Health (NIH), respectively.

CD4⁺ T Cell Isolation and In Vitro Differentiation.

Mice were anesthetized with a single intraperitoneal (IP) dose of pentobarbital sodium. Following cervical dislocation the spleens were aseptically removed and a single-cell suspension was obtained by gently pressing spleens through a 40 μm nylon mesh cell strainer (BD Falcon, San Jose, Calif.) placed inside one well of a six-well cell culture plate containing 3 ml of complete media (CM): 1640 RPMI supplemented with 10% FBS, 1% glutamine (100×) in 0.85% NaCl (Invitrogen, Carlsbad, Calif.) 1% antibiotic-antimycotic (100×) liquid: 10,000 units penicillin (base), 10,000 μg streptomycin, 25 μg amphotericin B/ml utilizing penicillin G (sodium salt), streptomycin sulfate and amphotericin B as Fungizone® Antimycotic in 0.85% saline (Invitrogen, Carlsbad, Calif.). The single-cell suspension was transferred and re-filtered through the mesh nylon cell strainer into a 50-ml conical vial (BD Falcon, San Jose, Calif.). The single well is washed thoroughly with an additional 3 ml of supplemented complete media and also filtered through the mesh cell strainer. Isolated splenocytes were collected by centrifugation at 1200 rpm for 5 min at 4° C. The red blood cells were lysed after re-suspending cells in 5 ml of ACK lysing buffer for 3 min at room temperature (RT). The buffer was neutralized with 5 ml of complete media. The resulting splenocytes were passed through a second 40 μm nylon mesh cell strainer and washed with complete media, pelleted and resuspended in 10 mls of complete media. The total cell number was determined, the cells were washed again and resuspended in 90 μl of degassed labeling buffer (solution containing PBS (phosphate buffered saline), pH 7.2, 0.5% BSA (bovine serum albumin) and 2 mM EDTA (ethylenediaminetetraacetic acid)) and 10 μl of CD4′ (L3T4) microbeads (Miltenyi Biotec, Auburn, Calif.) per 10⁷ total cells. The splenocytes were incubated on ice for 30 minutes. Subsequently, cells were washed with 10 ml of labeling buffer, spun at 1200 rpm for 5 min and resuspended in 500 μl of labeling buffer. The splenocytes were added to a prepared MACS LS column. After washing the column 3 times with 3 ml of labeling buffer, the column was removed from the magnetic field, 5 ml of buffer was added to the column and the CD4′ T cells were eluted from the column with the supplied plunger. The cells are counted, pelleted and resuspended in complete media at a concentration of 4×10⁶ cells per 10 ml. The purified CD4+ T cells were incubated in complete media. For T_{H}1 polarizing conditions, cells were cultured with: IL-12 (2 ng), IFN-γ (100 U) and anti-IL-4 (11B11, 10 ug) and for T_{H}2 polarizing conditions, the cells were cultured with: IL-4 (200 U), IL-6 (100 U), anti-IFN-γ(AN18, 5 ug) and anti-IL-12 (clone C17.8, 2 μg). All T cells were stimulated twice (day 0 and day 8) with 5 μg/ml plate-bound CD3e antibody (clone 145-2C11, BD Pharmingen), 5 μg/ml soluble CD28 antibody (clone 37.51, BD Pharmingen) and IL-2 (20 U) in flat-bottom 96-well cell culture plates (Corning) in tandem with polarizing conditions. Naïve T cells were not stimulated and total RNA was extracted immediately. Supernatants were collected for IL-4 and IFN-γ analysis.

Total RNA Isolation.

The CD4′ T cells (naïve and differentiated subsets) were homogenized in Trizol® Reagent using 1 ml of reagent per 5−10×10⁶ cells (Invitrogen, Carlsbad, Calif.) and either frozen at −80° C. for later extraction or immediate extraction of total RNA by adding chloroform per 1 ml of Trizol® Reagent. The RNA was precipitated from the colorless aqueous phase using 0.5 ml of isopropyl alcohol per 1 ml Trizol® Reagent used. The precipitated RNA was collected by centrifugation at 12,000×g for 10 min at 4° C. The RNA was washed with 1 ml 75% ethanol per 1 ml Trizol® Reagent used. The RNA was re-dissolved in nuclease-free water. RNA isolated from freshly sorted CD4′ naïve (˜18 million cells) or T_{H}1 and T_{H}2 cell differentiation experiments from material pooled from two independent 96-well plates each with ˜18 million cells.

Isolation and Enrichment of Small RNAs.

The small RNA fraction (<200 nt) was isolated from the CD4′ T cells with the Pure Link™ miRNA Isolation Kit (Invitrogen, Carlsbad, Calif.). To completely dissociate the nucleoprotein complexes the cells were lysed using 1 ml of Trizol® Reagent per 5−10×10⁶ cells and incubated at room temperature for 5 min. Per 1 ml of Trizol® Reagent 200 μl of cholorofom was added. The total lysate was shaken by hand for 15 seconds and incubated for 2-3 min. The mixture was centrifuged at 12,000×g at 4° C. for 15 min and separated into a lower phenol-chloroform phase, interphase and colorless upper aqueous phase. The upper aqueous phase containing the RNA was collected and mixed with 100% ethanol to a final concentration of 35%. The lysate-ethanol mixture was added to a Spin Cartridge (provided in the kit) and centrifuged at 12,000×g for 1 min at room temperature. The flow-through was retained and mixed with ethanol for a final concentration of 70%. The mixture was then added to a second Spin Cartridge and centrifuged at 12,000×g for 1 min at room temperature. The flow-through was discarded and the column-bound small RNA molecules were washed twice with 500 μl Wash Buffer (provided in the kit) and centrifuged the twice at 12,000×g for 1 min at room temperature and the flow-through was discarded. To remove any residual Wash Buffer the Spin Cartridge is centrifuged for 2-3 min at maximum speed at room temperature. The RNA is eluted after added 50 μl of sterile, RNAse-free water to the Spin Cartridge, incubated at room temperature for 1 min and collected at maximum speed for 1 min at room temperature. The RNA was stored at 80° C. or immediately submitted to the Microarray Core Facility (Baylor College of Medicine, Houston, Tex.) to assess quality and concentration. RNA quality of all samples was determined on an Aglient 2100 Bioanalyzer (quality parameters: ribosomal RNA concentration, DNA contamination, RNA integrity, and overall quality, Quantum Analytics, Inc., Foster City, Calif.) and the concentration was also measured by Nanodrop ND-1000 Spectrophotometer (Thermo Scientific, Wilmington, Del.). Purified RNA was kept in nuclease-free water at −80° C.

RNA End Modification and Amplification (REMA).

Denaturing 15% TBE-UREA polyacrylamide gels (Invitrogen, Carlsbad, Calif.) were used to further isolate short RNAs. An equal volume of enriched short RNA samples (10 μl containing 2-5 mg RNA) from naïve, T_{H}1 and T_{H}2 samples and gel loading buffer (Invitrogen, Carlsbad, Calif.) was heated to 65° C. for 5 min and loaded onto the gel. A 10 base pair DNA ladder (Invitrogen, Carlsbad, Calif.) was loaded into an additional well of the gel. After running the gel for 1 hr at 200V the short RNA bands (15-65 nt) that corresponded to 5-55 base pairs on the DNA ladder were excised from the gel, extracted from the polyacrylamide, precipitated and washed. In separate reactions, synthetic RNA adapter oligonucleotides were added to the 5′ (5′ GUU CAG AGU UCU ACA GUC CGA CGA UC 3′ (SEQ ID NO:280)) and 3′(5′ p-UCG UAU GCC GUC UUC UGC UUG-idT 3′ (SEQ ID NO:281)) ends of the short RNAs in the presence of RNase inhibitor buffer and ATP. To remove the un-ligated adapter sequences after the addition of the 5′ adapter, the ligated short RNAs were gel purified on a 15% polyacrylamide gel (Invitrogen, Carlsbad, Calif.) and excised from the gel based on the DNA ladder bands corresponding to 30-90 base pairs (RNA equivalent 40-100 nt). Similar steps were performed to ligate and gel purify the 3′ adapter to the ligated 5′ adapter RNA. The RNA was excised from a 10% TBE-Urea gel based on the DNA ladder bands corresponding to 50-120 base pairs (RNA equivalent 60-130 nt). Using a 3′ adapter sequence specific primer (5′ CAA GCA GAA GAC GGC ATA CGA 3′ (SEQ ID NO:325)). The resulting ligated short RNA sequences were reverse transcribed and PCR amplified for 15 cycles using 5′ and 3′ adapter specific primers (Forward primer-5′ AAT GAT ACG GCG ACC ACC GAC AGG TT CAG AGT TCT ACA GTC CGA 3′ (SEQ ID NO:326); reverse primer-5′ CAA GCA GAA GAC GGC ATA CGA 3′ (SEQ ID NO:327)). The sequences were identified using Illumina-based Next Generation Sequencing.

Novel mRNA Discovery Strategy.

The unique sequences that did not map to known miRNA precursors were subjected to a novel miRNA discovery pipeline previously described (Creighton et al., 2009). The small RNA sequences were mapped to the whole genome and the sequences that map exactly are retained (including 100 bases flanking each side). These putative miRNA hairpin sequences are folded with Vienna package (Hofacker, 2009) and those structures that meet Ambros criteria are filtered for single-loop hairpins with the putative miRNA on one side of the hairpin and have a minimum free energy of <−25 kcal/mol (Ambros et al., 2003). The hairpins are then trimmed to include only the putative precursor. Subsequently, they are refolded and filtered again using Ambros criteria. The hairpins that are produced are considered novel miRNA hairpin precursors containing mature miRNA sequences found in the small-RNAome of a sample.

Mice and Allergen Challenge.

Female C57BL/6 mice between 4 and 7 weeks of age were anesthetized in an airtight chamber purged with a 3.2% isoflurane in oxygen vapor mixture for 10 minutes to achieve deep general anesthesia. Anesthetized mice received intranasally 45 mL chicken ovalbumin (22.5 mg) and 9 mL of protease derived from Aspergillus melleus (formerly A. oryzae; Sigma chemical company, St. Louis, Mo.; 7 mg) in PBS by applying droplets to the nares with a pipette. Allergen challenged mice received intranasal allergen on a schedule of every alternate day for eight total challenges

Quantitation of Allergic Lung Disease.

24 hours after the final allergen challenge, mice were anesthetized with etomidate and placed on a mechanical ventilator inside a custom-designed rodent plethysmograph. Airway hyperresponsiveness (AHR) was assessed by determining the change in respiratory system resistance (R_{RS}) induced by provocative challenge with graded intravenous acetylcholine (Ach; dose expressed as mg/g body weight) as described (Kheradmand et al., 2002). Bronchoalveolar lavage fluid (BALF) was collected by instilling and withdrawing 1.6 ml of sterile phosphate buffered saline (PBS) through the tracheal cannula in two aliquots of 0.8 ml. BALF total and differential cell counts were performed using a standard hemocytometer and H&E staining of cytospin slides as described (Kheradmand et al., 2002). Quantitation of cytokines from BAL fluid was performed by bead-assisted analysis (MILLIPLEX MAP Kit Mouse Cytokine/Chemokine Immunoassay; Millipore, Billerica, Mass., USA) using a Bioplex analyzer (BioRad, Hercules, Calif.) according to the manufacturers' protocols. CD4 T cells from spleen were isolated and total RNA was extracted as described for lung.

Results

Novel miRNAs from Mouse T Cells.

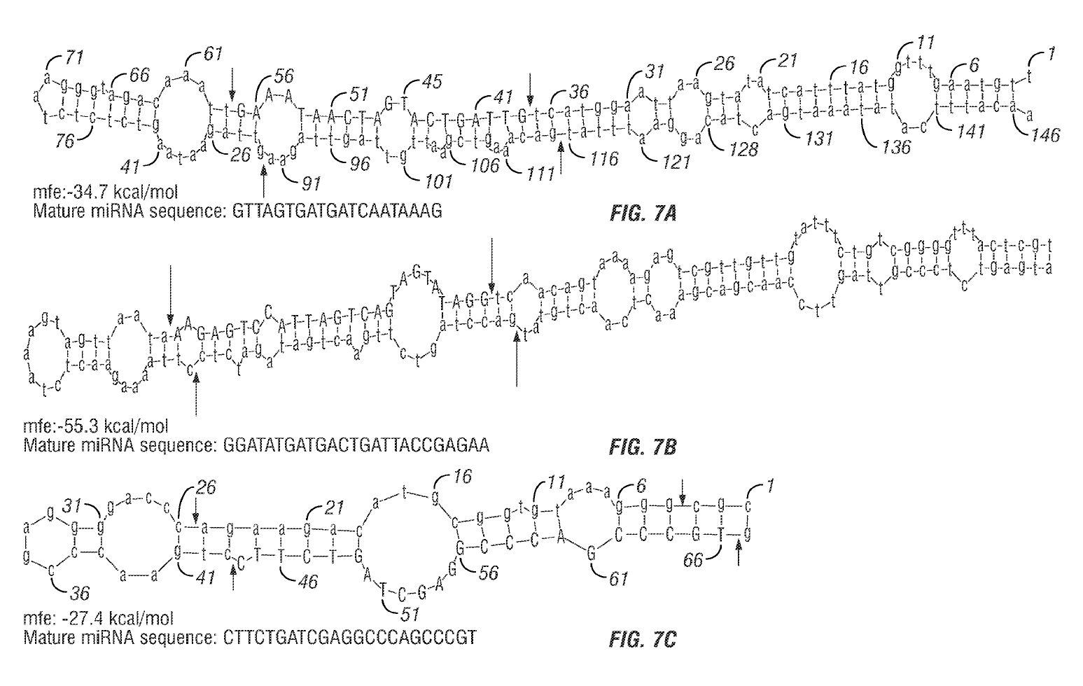

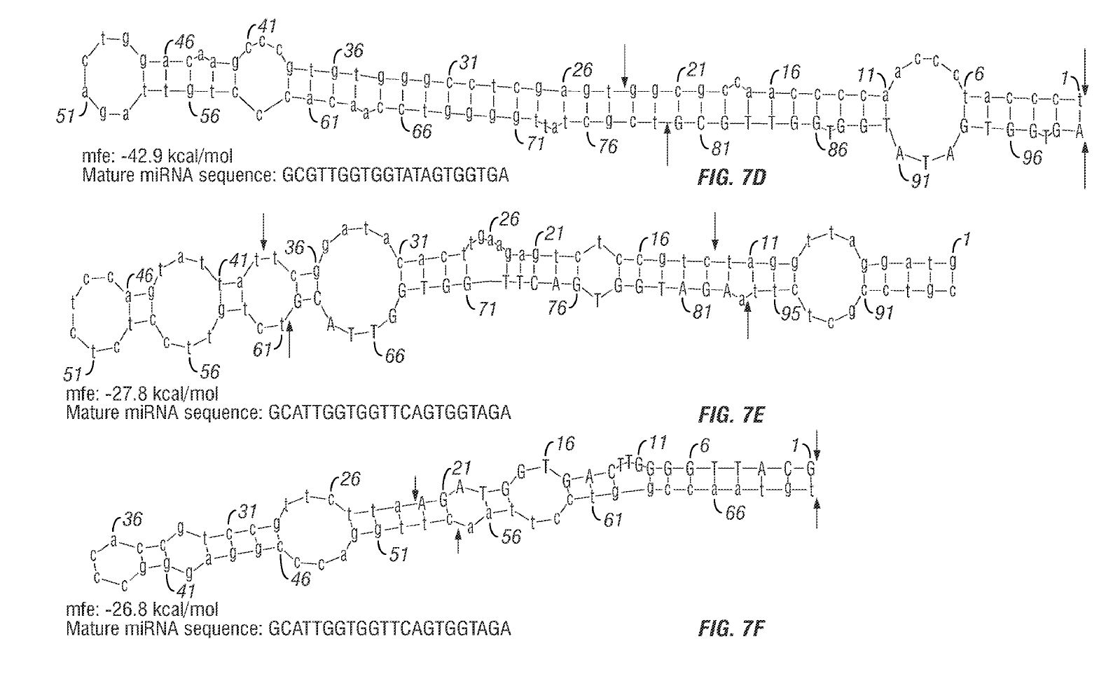

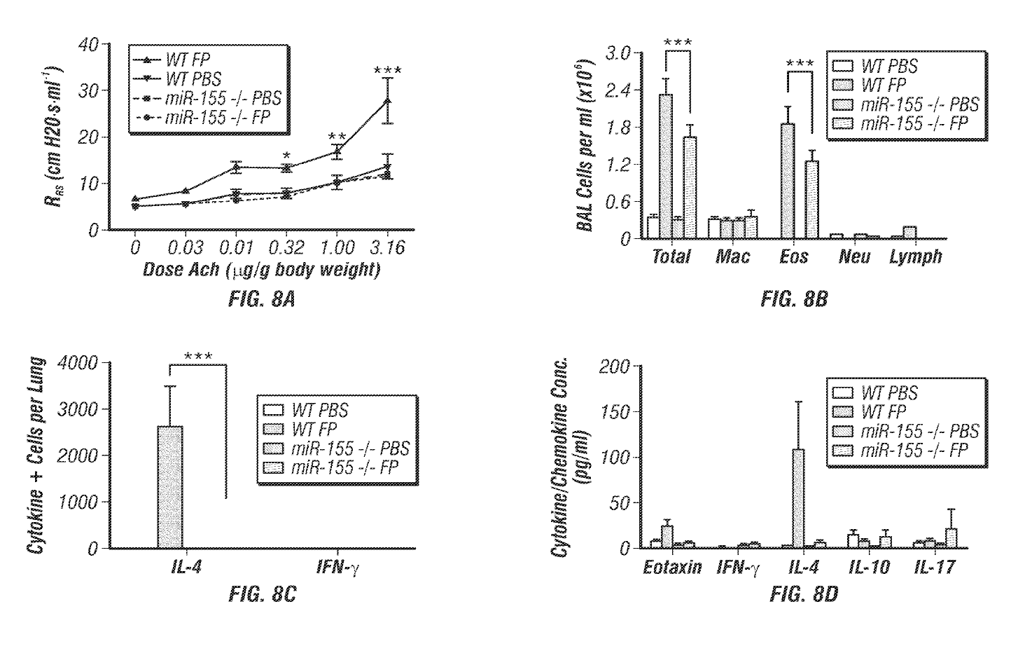

We sequences short RNAs from mouse T cells, comparing naïve to T_{H}1 and T_{H}2 cells. These findings revealed the presence of numerous miRNAs, especially members of the let-7 miRNA family (L M Batts, D B Corry, manuscript in preparation). Six novel miRNA were discovered and assigned according to the novel miRNA discovery platform (FIG. 7 and Methods). FIG. 7 illustrates six novel miRNAs placed in the context of their putative pre-miRNAs.

Mmu-mir-155 is Required for Expression of Allergic Lung Disease.

wild type and mice deficient in mmu-mir-155 were challenged intranasally with a fungal derived allergenic proteinase (FP) to determine the requirement of this miRNA for allergic lung disease. Mir-155^{-/-} mice failed to develop airway hyperreactivity as assessed by the change in respiratory system resistance in response to Ach challenge, whereas wild type mice developed robust airway hyperreactivity in comparison to PBS-challenged control animals (FIG. 2A). Furthermore, mir-155^{-/-} mice manifested reduced airway eosinophilia, an important marker of allergic inflammation, and failed to recruit to the lungs IL-4-secreting cells, including T_{H}2 cells (FIG. 2B, C). The lack of allergic cytokine secretion was further confirmed by analysis of bronchoalveolar lavage fluid, which showed robust IL-4 secretion into the in by wild type mice, but little or no IL-4 secretion in mir-155-deficient animals (FIG. 2D).

The data above indicates that mir-155 is required for expression of asthma-like disease in mice. These findings support the idea that inhibition of mir-155 may be therapeutic in persons with asthma.

### Example 4

### Chances in miRNA Expression in Lung Following Allergen Challenge

Asthma is an allergic disease that results in the obstruction of airways as a result of goblet cell hyperplasia and airway hyper-reactivity (AHR). Two novel miRNAs Asth-miR-1 and Asth-miR-2 were identified. Asth-miR-1 is predicted to target Toll-like receptor (TLR) adaptor TIRAP and IRAK which associates to activate NF-Kb, AP-1 and IRFs, and under some conditions, induce allergic lung disease. Asth-miR-2 is predicted to target ryanodine receptor 2 (RyR) that mediates Ca2+ release that induces airway smooth muscle contraction and bronchoconstriction. The integration of Asth-miR-1 and Asth-miR-1 2 with current therapies can potentially significantly enhance the efficacy and specificity of drugs used to combat asthma.

### Example 5

### MicroRNA Profiles of CD4+ Helper T Cell Subsets Methods Isolated CD4+

T cells from spleens of wildtype (WT) Balb/c mice (MACS system). Total RNA was isolated and from Naïve, TH1 and TH2 cells. Naïve cells were polarized using appropriate TH1 (anti-CD3, anti-CD28, IL-2, IFN-γ, anti-IL-4, IL-12) or TH2 (anti-CD3, anti-CD28, IL-2, anti-IFN-γ, IL-4, IL-6) skewing conditions for 10 days. Small RNA transcripts were sequenced using Next Generation Sequencing Technology (Solexa). mRNA expression was determined by microarray chip (Illumina).

Results

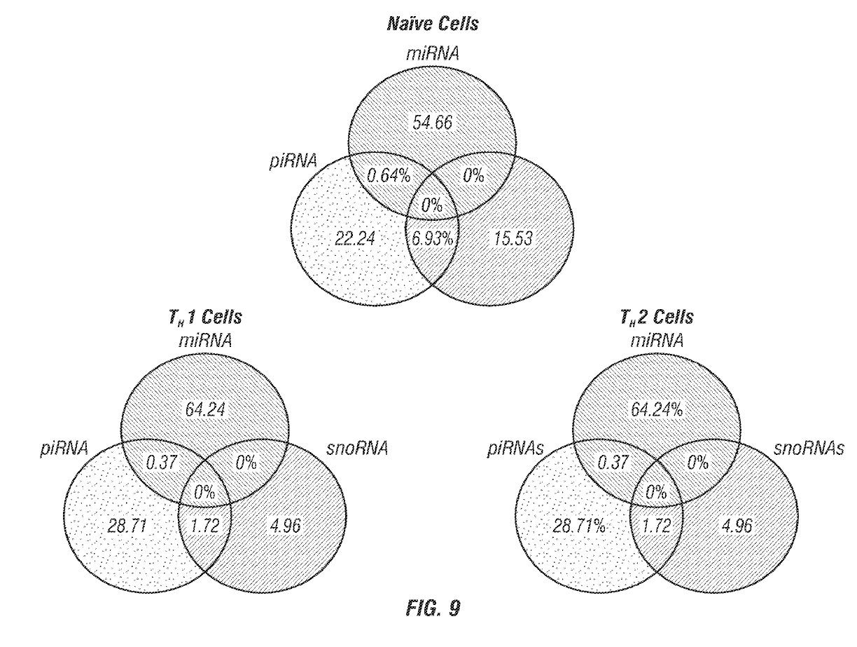

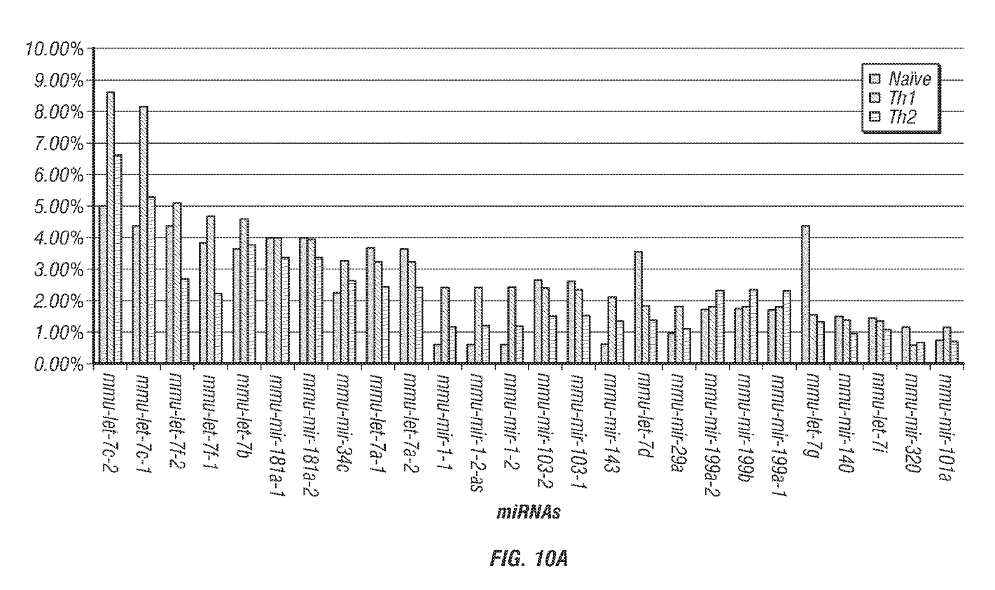

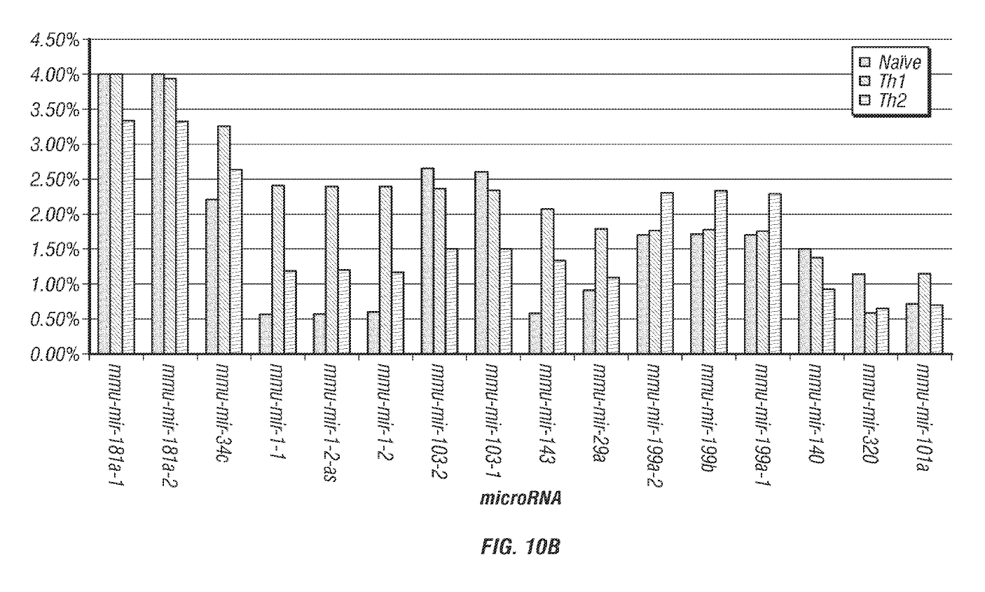

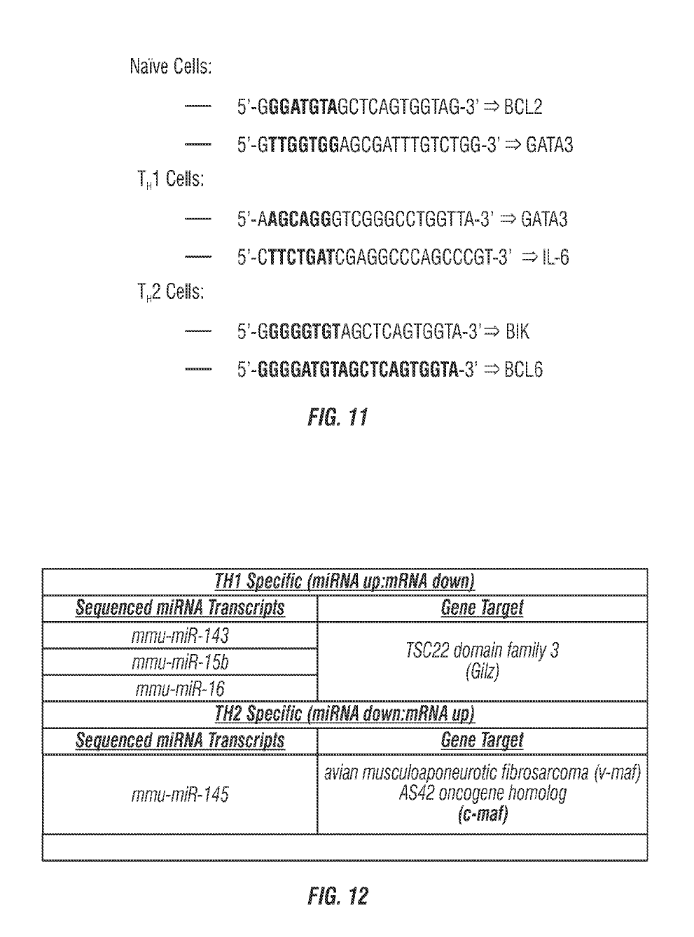

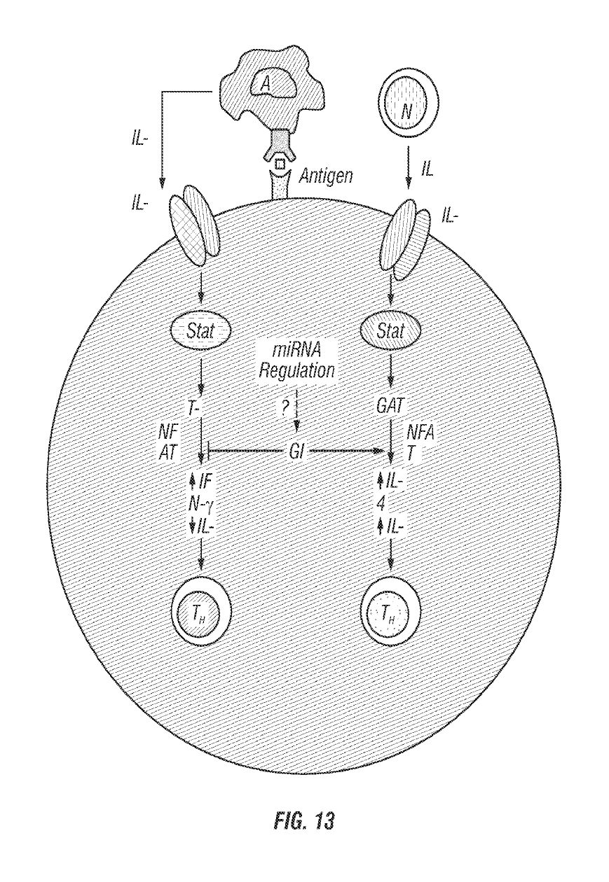

Results are depicted in FIG. 9, FIG. 10, FIG. 11, FIG. 12, and FIG. 13.

Small RNA Transcript Sequencing Reveals Novel mRNAs Naïve Cells:

5′-GGGATGTAGCTCAGTGGTAG-3′ (SEQ ID NO: 241) = BCL2
5′-GTTGGTGGAGCGATTTGTCTGG-3′ (SEQ ID NO: 242) =
GATA3
TH1 Cells:
5′-AAGCAGGGTCGGGCCTGGTTA-3′ (SEQ ID NO: 243) =
GATA3
5′-CTTCTGATCGAGGCCCAGCCCGT-3′ (SEQ ID NO: 244) =
IL-6
TH1 Cells:
5′-GGGGGTGTAGCTCAGTGGTA-3′ (SEQ ID NO: 245) = BIK

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,034,506
U.S. Pat. No. 5,235,033
U.S. Pat. No. 5,510,270
U.S. Pat. No. 5,556,752
U.S. Pat. No. 5,578,832
U.S. Pat. No. 6,268,490
U.S. Pat. No. 6,268,490
U.S. Pat. No. 6,770,748
U.S. Pat. No. 6,770,748
U.S. Patent Publn. 20020115080
U.S. Patent Publn. 20050107325
U.S. Patent Publn. 20050182005
Al-Shami et al., J. Exp. Med., 202:829-839, 2005.
Ambros et al., RNA, 9:277-279, 2003.
Ambros, Cell, 113(6):673-676, 2003.
Angel et al., Cell, 49:729, 1987a.
Angel et al., Cell, 49:729, 1987b.
Angkasekwinai et al., J. Exp. Med., 204:1509-1517, 2007.
Arima et al., J. Allergy Clin. Immunol., 109(6):980-7, 2002.
Atchison and Perry, Cell, 46:253, 1986.
Atchison and Perry, Cell, 48:121, 1987.
Ausubel et al., Current Protocols in Molecular Biology, Greene Publ. Assoc. Inc. & John Wiley & Sons, Inc., MA, 1997.
Ayroldi et al. Blood, 98(3):743-53, 2001.
Baichwal and Sugden, In: Gene Transfer, Kucherlapati (Ed.), Plenum Press, NY, 117-148, 1986.
Banerji et al., Cell, 27:299, 1981.
Banerji et al., Cell, 33(3):729-740, 1983.
Barnes et al., J. Biol. Chem., 272(17):11510-11517, 1997.
Bass, B Cold Spring Harb. Symp. Quant. Biol., 71:285-292, 2006.
Behm-Ansmant et al., Cold Spring Harb. Symp. Quant. Biol., 71:523-530, 2006.
Benvenisty and Neshif, Proc. Natl. Acad. Sci. USA, 83(24):9551-9555, 1986.
Berkhout et al., Cell, 59:273-282, 1989.
Bhaysar et al., Genomics, 35(1):11-23, 1996.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Boussiotis et al., Proc. Natl. Acad. Sci. USA, 90:11059-11063, 1993.
Braddock et al., Cell, 58:269, 1989.
Bragonzi et al., Gene Ther., 7:1753-1760, 2000.
Brennecke et al., Cell, 113(1):25-36, 2003.
Bulla and Siddiqui, J. Virol., 62:1437, 1986.
Calin et al., Proc. Natl. Acad. Sci. USA, 105:5166-5171, 2008.
Calin et al., Proc. Natl. Acad. Sci. USA, 99(24):15524-15529, 2002.
Camarlle et al., Immunobiology, 2005.
Campbell and Villarreal, Mol. Cell. Biol., 8:1993, 1988.
Campere and Tilghman, Genes and Dev., 3:537, 1989.
Campo et al., Nature, 303:77, 1983.
Carrington and Ambros, Science, 301(5631):336-338, 2003.
Celander and Haseltine, J. Virology, 61:269, 1987.
Celander et al., J. Virology, 62:1314, 1988.
Chang et al., Mol. Cell. Biol., 9:2153, 1989.
Chatterjee et al., Proc. Natl. Acad. Sci. USA, 86:9114, 1989.
Chen and Okayama, Mol. Cell. Biol., 7(8):2745-2752, 1987.
Chen et al., Mol. Microbiol., 53843-856, 2004.
Chen, Science, 303:2022-2025, 2004.
Chin and Werb, Development, 124:1519-1530, 1997.
Choi et al., J. Mol. Biol., 262(2):151-167, 1996.
Chu and Rana, Plos. Biology., 4:e210, 2006.
Chunn et al., J. Immunol., 167:4676-4685, 2001.
Coarfa and Milosavljevic, Pac. Symp. Biocomput., 102-13, 2008.
Cobb et al. J. Ex. Med., 2007.
Coffin, In: Virology, Fields et al. (Eds.), Raven Press, NY, 1437-1500, 1990.
Cohen et al., J. Cell. Physiol., 5:75, 1987.
Cormier et al., Mamm. Genome, 12(5):352-61, 2001.
Corry et al., J Exp Med., 183(1):109-17, 1996.
Corry et al., Mol. Med., 4:344-355, 1998.
Corry, Curr. Opin. Immunol., 11:610-614, 1999.
Costa et al., Mol. Cell. Biol., 8:81-90, 1988.
Coupar et al., Gene, 68:1-10, 1988.
Creighton et al., Brief Bioinform., 10:490-497, 2009.
Creighton et al., Rna, 14:2290-2296, 2008.
Cripe et al., EMBO J., 6:3745, 1987.
Culotta and Hamer, Mol. Cell. Biol., 9:1376-1380, 1989.
Dandolo et al., J. Virology, 47:55-64, 1983.
Davidson-Moncada et al., Ann NY Acad. Sci., 1183:183-94, 2010.
De Andres et al., BioTechniques 18:42044, 1995.
DeRisi Shalon et al., Genome Res., 6:639-645, 1996.
Deschamps et al., Science, 230:1174-1177, 1985.
Ding and Cantor, Proc. Natl. Acad. Sci. USA, 100:3059-3064, 2003.
Dostie et al., Rna-A Publication of the Rna Society, 9:180-186, 2003.
Drouin et al., J. Immunol., 169:5926-5933, 2002.
Dubensky et al., Proc. Natl. Acad. Sci. USA, 81:7529-7533, 1984.
Dunford et al., J. Immunol., 176:7062-7070, 2006.
Edbrooke et al., Mol. Cell. Biol., 9:1908-1916, 1989.
Edlund et al., Science, 230:912-916, 1985.
Eisenbarth et al., J. Exp. Med., 196:1645-1651, 2002.
Elmen et al., Nature, 452(7189):896-9, 2008.
Eulalio et al., Cell, 132:9-14, 2008.
Fahy et al., Curr. Opin. Pulm. Med., 6:15-20, 2000.
Fechheimer et al., Proc Natl. Acad. Sci. USA, 84:8463-8467, 1987.
Feng and Holland, Nature, 334:6178, 1988.
Ferguson et al., Proc. Natl. Acad. Sci. USA, 97(11):6049-6054, 2000.
Firak and Subramanian, Mol. Cell. Biol., 6:3667, 1986.
Fodor et al., Science, 251:767-773, 1991.
Foecking and Hofstetter, Gene, 45(1):101-105, 1986.
Fraley et al., Proc. Natl. Acad. Sci. USA, 76:3348-3352, 1979.
Franz et al., Cardioscience, 5(4):235-43, 1994.
Friedman et al., Genome Res., 19:92-105, 2009.
Fujita et al., Cell, 49:357, 1987.
Fukumura et al., Nucl. Acids. Res., 31(16):e94, 2003.
Furuno et al., Genes & Immunity, 9:302-308, 2008.
GeurtsvanKessel et al., J. Exp. Med., 205:1621-1634, 2008.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gillies et al., Cell, 33:717, 1983.
Giordano et al., Am. J. Pathol., 162:521-531, 2003.
Gloss et al., EMBO J., 6:3735, 1987.
Godbout et al., Mol. Cell. Biol., 8:1169, 1988.
Godfrey et al., J. Molec. Diagn., 2: 84-91, 2000.
Goodbourn and Maniatis, Cell, 41(2):509-520, 1985.
Goodbourn et al., Cell, 45:601, 1986.
Gopal, Mol. Cell. Biol., 5:1188-1190, 1985.
Gopal-Srivastava et al., J. Mol. Cell. Biol. 15(12):7081-7090, 1995.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al., J. Gen. Virl., 36(1):59-74, 1977.
Greene et al., Immunology Today, 10:272, 1989.
Grosschedl and Baltimore, Cell, 41:885, 1985.
Grunhaus and Horwitz, Seminar in Virology, 3:237-252, 1992.
Grunig et al., Science, 282(5397):2261-2263, 1998.
Gu et al., PloS ONE, 3:e2548, 2008.
Habig et al., Mol. Cell, 25:792-793, 2007.
Hammell et al., Proc. Natl. Acad. Sci. USA, 106(44):18668-73, 2009.
Harland and Weintraub, J. Cell Biol., 101(3):1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hilton et al., Proc. Natl. Acad. Sci. USA, 93:497-501, 1996.
Hirochika et al., J. Virol., 61:2599, 1987.
Hirsch et al., Mol. Cell. Biol., 10:1959, 1990.
Hod, Biotechniques, 13:852 854, 1992.
Hofacker, In: RNA secondary structure analysis using the Vienna RNA package, John Wiley & Sons, Inc., 2009.
Holbrook et al., Virology, 157:211, 1987.
Horlick and Benfield, Mol. Cell. Biol., 9:2396, 1989.
Horwich et al. J. Virol., 64:642-650, 1990.
Hou et al., Science, 265:1701-1706, 1994.
Huang et al., Cell, 27:245, 1981.
Huang et al., J Immunol., 155(5):2688-94, 1995.
Hug et al., Mol. Cell. Biol., 8:3065-3079, 1988.
Hwang et al., Mol. Cell. Biol., 10:585, 1990.
Idzko et al., Nat. Med., 13:913-919, 2007.
Iliopoulos et al., Cell, 139(4):693-706, 2009.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Inoue et al., J. Experim. Med., 201:73-82, 2005.
Jaffar et al., J. Immunol., 163:6283-6291, 1999
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9(8):3393-3399, 1989.
Jones and Shenk, Cell, 13:181-188, 1978.
Kadesch and Berg, Mol. Cell. Biol., 6:2593, 1986.
Kalafus et al. Genome Res., 14(4):672-8, 2004.
Karin et al., Mol. Cell. Biol., 7:606, 1987.
Kasaian et al., Am. J. Respir. Cell Mol. Biol., 36(3):368-76., 2007.
Katinka et al., Cell, 20:393, 1980.
Katinka et al., Nature, 290:720, 1981.
Kawamoto et al., Genome Res., 12:1305-1312, 1999.
Kawamoto et al., Mol. Cell. Biol., 8:267, 1988.
Keane-Myers et al., J. Immunol., 158:2042-2049, 1997.
Keller and Lowenstein, Semin. Respir. Crit. Care Med., 23:317-330, 2002.
Kelly et al., J. Cell Biol., 129(2):383-396, 1995.
Kheradmand et al., J. Immunol., 169:5904-5911, 2002.
Kiledjian et al., Mol. Cell. Biol., 8:145, 1988.
Kim et al., Nat. Rev. Mol. Cell. Biol., 10(2):126-39, 2009.
Kimura et al., Dev. Growth Differ. 39(3):257-265, 1997.
Kimzey et al., J. Immunol., 173:632-664, 2004.
Kishikawa et al., J. Immunol., 167:4414-4420, 2001.
Kiss et al., J. Allergy Clin. Immunol., 120:334-342, 2007.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, Mol. Cell. Biol., 3:325, 1983.
Kriegler et al., Cell, 38:483, 1984a.
Kriegler et al., In: Cancer Cells 2/Oncogenes and Viral Genes, Van de Woude et al. eds, Cold Spring Harbor, Cold Spring Harbor Laboratory, 1984b.
Krutzfeldt et al., Nature, 438(7068):685-689, 2005.
Kuhl et al., Cell, 50:1057, 1987.
Kunz et al., Nucl. Acids Res., 17:1121, 1989.
Kuperman et al., J. Exp. Med., 187(6):939-48, 1998.
Lanford et al., Science, 327(5962):198-201, 2010.
LaPointe et al., Hypertension 27(3 Pt 2):715-22, 1996.
LaPointe et al., J. Biol. Chem., 263(19):9075-8, 1988.
Larsen et al., Proc. Natl. Acad. Sci. USA, 83:8283, 1986.
Laspia et al., Cell, 59:283, 1989.
Latimer et al., Mol. Cell. Biol., 10:760, 1990.
Lee and Ambros, Science, 294(5543):862-864, 2001.
Lee et al., Nature, 294:228, 1981.
Lee et al., Nucleic Acids Res., 12:4191-206, 1984.
Li et al., Cell, 129(1):147-161, 2007.
Liang and Pardee, Science, 257:967-971, 1992.
Lin et al., Mol. Cell. Biol., 10:850, 1990.
Lockhart et al., Nat. Biotechnol., 14(13):1675-1680, 1996.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Lytle et al., Proc. Natl. Acad. Sci. USA, 104(23):9667-72, 2007.
Macejak and Sarnow, Nature, 353:90-94, 1991.
Majors and Varmus, Proc. Natl. Acad. Sci. USA, 80:5866, 1983.
Maskos and Southern, Nuc. Acids. Res., 20:1679-1, 684, 1992.
Matthews et al., Blood, 85:38-42, 1995.
McArthur and Raulet, J. Exp. Med., 178:1645-1653, 1993.
McNeall et al., Gene, 76:81, 1989.
Metzler et al., Genes Chromosomes Cancer, 39:167-169, 2004.
Michael et al., Mol. Cancer. Res., 1:882-891, 2003.
Miksicek et al., Cell, 46:203, 1986.
Mordacq and Linzer, Genes and Dev., 3:760, 1989.
Moreau et al., Nucl. Acids Res., 9:6047, 1981.
Moss et al., Biol. Chem., 271(49):31688-31694, 1996.
Muesing et al., Cell, 48:691, 1987.
Nakanishi et al., Proc. Natl. Acad. Sci. USA, 98(9):5175-80, 2001.
Neuberger et al., Nucleic Acids Res., 16(14B):6713-6724, 1988.
Ng et al., Nuc. Acids Res., 17:601, 1989.
Nicolau and Sene, Biochim. Biophys. Acta, 721:185-190, 1982.
Nicolau et al., Methods Enzymol., 149:157-176, 1987.
Ohshima et al., Blood, 92:3338-3345, 1998.
Oliphant et al., In: Discovery of Markers for Disease (Supplement to Biotechniques), June 2002.
Omitz et al., Mol. Cell. Biol., 7:3466, 1987.
Ondek et al., EMBO J., 6:1017, 1987.
Palmiter et al., Cell, 29:701, 1982.
Parker and Barnes, Methods Mol. Biol., 106:247-83, 1999.
Pasquinelli and Ruvkun, Ann. Rev. Cell Dev. Biol., 18:495-513, 2002. PCT Appln. WO/0071096
Pease et al., Proc. Natl. Acad. Sci. USA, 91:5022-5026, 1994.
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Pfaffl et al., Nucleic Acids Res., 30:e36, 2002.
Pfeffer et al., Science, 304:734-736, 2004.
Picard and Schaffner, Nature, 307:83, 1984.
Pinkert et al., Genes and Dev., 1:268, 1987.
Polikepahad et al., J. Immunol., 182:136.22, 2009.
Ponta et al., Proc. Natl. Acad. Sci. USA, 82:1020, 1985.
Potter et al., Proc. Natl. Acad. Sci. USA, 81:7161-7165, 1984.
Pouladi et al., Am. J. Respir Cell Mol. Biol., 30(1):84-90, 2004.
Queen and Baltimore, Cell, 35:741, 1983.
Quinn et al., Mol. Cell. Biol., 9:4713, 1989.
Redecke et al., J. Immunol., 172:2739-2743, 2004.
Redondo et al., Science, 247:1225, 1990.
Rehmsmeier et al., Rna, 10:1507-1517, 2004.
Reid et al., Genome Res., 18:1571-1581, 2008.
Reinhart et al., Nature, 403:901-906, 2000.
Reisman and Rotter, Mol. Cell. Biol., 9:3571, 1989.
Remington's Pharmaceutical Sciences, 15^{th} Ed., 1035-1038 and 1570-1580, 1990.
Resendez Jr. et al., Mol. Cell. Biol., 8:4579, 1988.
Ridgeway, In: Vectors: A Survey of Molecular Cloning Vectors and Their Uses, Rodriguez et al. (Eds.), Stoneham: Butterworth, 467-492, 1988.
Rippe et al., Mol. Cell. Biol., 9(5):2224-22277, 1989.
Rippe, et al., Mol. Cell. Biol., 10:689-695, 1990.
Rittling et al., Nucl. Acids Res., 17:1619, 1989.
Rudic et al., PLoS Biol., 2(11):e377, 2004.
Rupp and Locker, Lab Invest., 56:A67, 1987.
Russell et al., Science, 262:1880-1883, 1993.
Saldanha, Bioinformatics, 20(17):3246-8, 2004.
Salek-Ardakani et al., J. Exp. Med., 198:315-324, 2003.
Schaffner et al., J. Mol. Biol., 201:81, 1988.
Schena et al, Science, 270:467-470, 1995.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Soutschek et al., Nature, 432:173-178, 2004.
Spalholz et al., Cell, 42:183, 1985.
Spandau and Lee, J. Virology, 62:427, 1988.
Spandidos and Wilkie, EMBO J., 2:1193, 1983.
Specht et. al., Am. J. Pathol., 158: 419-29, 2001.
Stein et al., Nucleic Acids Res., 38(1):e3, 2010.
Stephens and Hentschel, Biochem. J., 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Takeyama et al., Proc. Natl. Acad. Sci. USA, 96:3081-3086, 1999.
Tamada et al., Nat. Med., 6:283-289, 2000.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Treisman, Cell, 42:889, 1985.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tur-Kaspa et al., Mol. Cell. Biol., 6:716-718, 1986.
Tyndall et al., Nuc. Acids. Res., 9:6231, 1981.
Vasseur et al., Proc. Natl. Acad. Sci. USA, 77:1068, 1980.
Vasudevan et al., Science, 318(5858):1931-4, 2007.
Velculescu et al., Cell, 88:243-51, 1997.
Velculescu et al., Science, 270:484-487, 1995.
Viswanathan et al., Nat. Genet., 41(7):843-8, 2009.
Viswanathan et al., Science, 320(5872):97-100, 2008.
Wahlestedt et al., Proc. Natl. Acad. Sci. USA, 97(10):5633-8, 2000.
Wang and Calame, Cell, 47:241, 1986.
Webb et al., J Biol. Chem., 276(45):41969-76, 2001.
Weber et al., Cell, 36:983, 1984.
Weis et al., Trends in Genetics, 8:263 264, 1992.
Wills-Karp, Annu. Rev. Immunol., 17:255-281, 1999.
Winoto and Baltimore, Cell, 59:649, 1989.
Wong et al., Gene, 10:87-94, 1980.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Xiang et al., Nat. Med., 13:862-867, 2007.
Xu et al., Biol. Reprod., 79:51-57, 2008.
Xu et al., Curr. Biol., 13(9):790-795, 2003.
Yamauchi-Takihara et al., Proc. Natl. Acad. Sci. USA, 86(10):3504-8, 1989.
Yang and Russell, Proc. Natl. Acad. Sci. USA, 87:4144-4148, 1990.
Yang et al., Genome Res., 11:1888-1898, 2001.
Yutzey et al. Mol. Cell. Biol., 9:1397, 1989.
Zhao et al., Cell, 129:303-317, 2007.
Zheng and Flavell, Cell, 89:587-596, 1997.
Zheng et al., J. Immunol., 162:4983-4990, 1999.
Zhou et al., Nat. Immunol., 6:1047-1053, 2005.
Ziober and Kramer, J. Bio. Chem., 271(37):22915-22, 1996.
Zuker and Jacobson, RNA, 4:669-679. 1998.
Zurawski et al., Embo J., 12:2663-2670, 1993.

1. A method for detecting an allergic or inflammatory lung disease, comprising assessing the level of one or more microRNAs (miRNAs) in a biological sample, wherein the level of the one or more miRNAs in the biological sample compared to a reference level of the one or more miRNAs is indicative of allergic or inflammatory lung disease.

2. The method of claim 1, wherein at least one of the one or more miRNAs comprises:
(i) mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, mir-712, Asth-miR-1, or Asth-miR-2;
(ii) mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, or mir-467c;
(iii) a sequence that has at least 80% sequence identity to a sequence as set forth in (i);
(iv) a sequence that has at least 80% sequence identity to a sequence as set forth in (ii);
(v) the complement of a sequence as set forth in (i) or (iii); or
(vi) the complement of a sequence as set forth in (ii) or (iv);
wherein a decrease in the expression level of one or more miRNAs from group (i), (iii) or (v), or an increase in the expression level of one or more miRNAs from group (ii), (iv) or (vi) in the biological sample compared to a reference level of the one or more miRNAs is indicative of allergic or inflammatory lung disease.

3.-14. (canceled)

15. A biochip comprising an isolated nucleic acid comprising:
(i) mir-147, mir-135a, mir-135b, mir-683, mir-130b, mir-1, mir-615-Sp, mir-142-3p, mir-689, mir-130b, mir-155, mir-146b, mir-18b, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-770-5p, mir-483, mir-193, mir-296-5p, mir-715, or mir-712;
(ii) a sequence that has at least 80% sequence identity to a sequence as set forth in (i);
(iii) the complement of a sequence as set forth in (i) or (ii); or
(iv) a nucleic acid sequence comprising at least 10 contiguous nucleic acids of Asth-miR-1 (SEQ ID NO:187), Asth-miR 2 (SEQ ID NO:189), or Asth-miR-5 (SEQ ID NO:195); attached to said biochip.

16. (canceled)

17. A method of inhibiting a target gene in a cell, comprising contacting the cell with a nucleic acid in an amount sufficient to inhibit expression of the target gene, wherein the nucleic acid comprises:
(i) mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712;
(ii) a sequence that has at least 80% sequence identity to a sequence as set forth in (i);
(iii) the complement of a sequence as set forth in (i) or (ii); or
(iv) a nucleic acid sequence comprising at least 10 contiguous nucleic acids of Asth-miR-1 (SEQ ID NO:187), Asth-miR 2 (SEQ ID NO:189), or Asth-miR-5 (SEQ ID NO:195).

18.-25. (canceled)

26. A method of treating or preventing exacerbation of an allergic lung disease in a subject, comprising administering to said subject a pharmaceutically effective amount of a composition comprising a nucleic acid comprising:
(i) mir-681, mir-880, mir-1190, mir-709, mir-671-3p, mir-1196, mir-667, mir-483*, mir-331-3p, mir-743a, mir-485, mir-30c-1*, mir-770-5p, mir-483, mir-193, mir-296-5p, mir-715, mir-712, Asth-miR-1, or Asth-miR-2; or
(ii) a nucleic acid which selectively binds or inhibits one or more of: mir-147, mir-135a, mir-135b, mir-683, mir-130b, mir-1, mir-615-5p, mir-142-3p, mir-689, mir-130b, mir-155, mir-146b, mir-18b, mir-501-5p, mir-1191, mir-421, mir-146b*, mir-717, or mir-467c.

27. The method of claim 26, wherein the nucleic acid is a group (ii) nucleic acid, and wherein the nucleic acid is chemically modified or comprises a nucleotide analog.

28. The method of claim 27, wherein the nucleic acid is selected from the group consisting of (5′-AACTATACAACCTACTACCTCA-3′ (SEQ ID NO:246)), (5′-AACTATACAACCTCCTACCTCA-3′ (SEQ ID NO:247)), and (5′-CAACCTACTACCTC-3′ (SEQ ID NO:248)).

29. The method of claim 28, wherein the nucleic acid is a LNA.

30. The method of claim 26, wherein the subject is a mammal.

31. The method of claim 30, wherein the mammal is a human.

32. The method of claim 30, wherein the allergic lung disease is asthma, hay fever, or hypersensitivity pneumonitis.

33. The method of claim 32, wherein the allergic lung disease is asthma.

34. The method of claim 26, wherein said nucleic acid comprises a phosphoramidate linkage, a phosphorothioate linkage, a phosphorodithioate linkage, or an O-methylphosphoroamidite linkage.

35. The method of claim 26, wherein said nucleic acid comprises one or more nucleotide analogs.

36. The method of claim 26, further comprising administering to the subject one or more secondary forms of therapy for the treatment or prevention of allergic lung disease.

37. The method of claim 36, wherein the secondary form of therapy is selected from the group consisting of a corticosteroid, a beta-2 adrenergic receptor agonist, a leukotrine modifier, an anti-immunoglobulin E (IgE) antibody, or a mast cell stabilizing agent.

38. The method of claim 26, wherein said nucleic acid is comprised in a vector.

39. The method of claim 38, wherein said vector is a viral vector.

40. The method of claim 39, wherein said viral vector is an adenovirus, an adeno-associated virus, a lentivirus, or a herpes virus.

41. The method of claim 26, wherein said vector comprises a lipid.

42. The method of claim 41, wherein said lipid is comprised in a liposome.

43. The method of claim 26, wherein the pharmaceutically effective amount of said composition is administered via an aerosol, topically, locally, intravenously, intraarterially, intramuscularly, by lavage, or by injection into the thoracic cavity.

44. A kit comprising a biochip as set forth in claim 15 and one or more sealed containers.

45. (canceled)

46. A kit comprising a sealed container comprising a nucleic acid, wherein said nucleic acid comprises:
(i) mir-147, mir-135a, mir-135b, mir-683, emir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c, mir-681, mir-880, mir-1190, mir-709, mir-671-3p, mir-1196, mir-667, mir-452, mir-483*, mir-743a, mir-485, mir-30c-1*, mir-770-5p, mir-483, mir-193, mir-296-5p, mir-715, or mir-712;
(ii) a sequence that has at least 80% sequence identity to a sequence as set forth in (i);
(iii) the complement of a sequence as set forth in (i) or (ii); or
(iv) a nucleic acid sequence comprising at least 10 contiguous nucleic acids of Asth-miR-1 (SEQ ID NO:187), Asth-miR 2 (SEQ ID NO:189), or Asth-miR-5 (SEQ ID NO:195).

47.-49. (canceled)

50. A kit comprising a sealed container comprising a set of primers specific for transcription or reverse transcription of a nucleic acid sequence, wherein said nucleic acid sequence comprises:
(i) mir-147, mir-135a, mir-135b, mir-683, mir-130b, mir-1, mir-615-5p, mir-142-3p, 689, mir-130b, mir-155, mir-146b, mir-18b, mir-340-5p, mir-501-5p, mir-1191, mir-421, mir-146b*, mir-717, mir-467c, mir-681, mir-880, mir-1190, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712;
(ii) a sequence that has at least 80% sequence identity to a sequence as set forth in (i);
(iii) the complement of a sequence as set forth in (1) or (ii); or
(iv) a nucleic acid sequence comprising at least 10 contiguous nucleic acids of Asth-miR-1 (SEQ ID NO:187), Asth-miR 2 (SEQ ID NO:189), or Asth-miR-5 (SEQ ID NO:195).

51. A method of treating an allergic or inflammatory lung disease in a subject comprising administering to the subject a let-7 miRNA inhibitor.

52. The method of claim 51, wherein the let-7 miRNA inhibitor is selected from the group consisting of siRNA, an antisense oligonucleotide, a locked nucleic acid (LNA), an antisense RNA, and a plasmid expressing an antisense RNA.

53. The method of claim 52, wherein the let-7 miRNA inhibitor is an LNA.

54. The method of claim 53, wherein the LNA comprises:
(i) (5″-AACTATACAACCTACTACCTCA-3′ (SEQ ID NO:246)), (5′-AACTATACAACCTCCTACCTCA-3′ (SEQ ID NO:247)), or (5′-CAACCTACTACCTC-3′ (SEQ ID NO:248));
(ii) a sequence having at least 80% sequence identity to a sequence as set forth in (i); or
(iii) the complement of a sequence as set forth in (i) or (ii).

55. The method of claim 52, wherein the let-7 miRNA inhibitor is administered in a pharmaceutically acceptable composition.

56. The method of claim 55, wherein the let-7 miRNA inhibitor is administered orally, intravenously, via an aerosol, topically, locally, intravenously, intraarterially, intramuscularly, by lavage, or by injection into the thoracic cavity.

57. The method of claim 56, wherein the subject is a mouse, a rat, a rodent, a cat, a horse, a goat, a sheep, a cow, a rabbit, a primate, or a human.

58. An isolated nucleic acid comprising:
(i) (5′-AACTATACAACCTACTACCTCA-3′, SEQ ID NO:246), (5′-AACTATACAACCTCCTACCTCA-3′ SEQ ID NO:247), or (5′-CAACCTACTACCTC-3′ SEQ ID NO:248);
(ii) a sequence having at least 80% sequence identity to (5′-AACTATACAACCTACTACCTCA-3′ SEQ ID NO:246), (5′-AACTATACAACCTCCTACCTCA-3′ SEQ ID NO:247), or (5′-CAACCTACTACCTC-3′ SEQ ID NO:248); or
or (iii) the complement of a sequence as set forth in (i) or (ii);
wherein the isolated nucleic acid can selectively bind a let-7 miRNA.

59.-65. (canceled)

66. A method of screening for a modulator of an allergic or inflammatory lung response comprising;
(a) contacting a lung cell with a candidate substance; and
(b) measuring the expression level of one or more microRNAs (miRNAs) in the lung cell;
wherein at least one of the one or more miRNAs comprises: mir-147, mir-135a, mir-135b, mir-683, mir-130b, mir-1, mir-615-5p, mir-142-3p, mir-689, mir-130b, mir-155, mir-146b, mir-18b, mir-340-5p, mir-50′-5p, mir-1191, mir-421, mir-146b*, mir-717, mir-467c, mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, or mir-712, Asth-miR-1 (SEQ ID NO:187), Asth-miR 2 (SEQ ID NO:189), or Asth-miR-5 (SEQ ID NO:195);
wherein an increase in the expression level of one or more of: mir-681, mir-880, mir-1190, mir-709, mir-67′-3p, mir-1196, mir-667, mir-452, mir-483*, mir-331-3p, mir-743a, mir-485, mir-30c-1*, mir-770-5p, mir-483, mir-193, mir-715, mir-712, Asth-miR-1, or Asth-miR-2 in the lung cell indicates that the modulator can inhibit an allergic or inflammatory lung response; and
wherein a decrease in the expression level of one or more of mir-147, mir-135a, mir-135b, mir-683, mir-130b, mir-1, mir-615-5p, mir-142-3p, mir-689, mir-130b, mir-155, mir-146b, mir-18b, mir-501-5p, mir-1191, mir-421, mir-146b*, mir-717, mir-467c in the lung cell indicates that the modulator can inhibit an allergic or inflammatory lung response.

67. A method of identifying a subject to receive an inhibitor of an allergic or inflammatory lung response comprising:
measuring the expression level of one or more microRNAs (miRNAs) in a lung cell from the subject; wherein at least one of the one or more miRNAs comprises: mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1 191, mir-421, mir-146b*, mir-717, mir-467c, mir-681, mir-880, mir-1190, mir-671-3p, mir-1196, mir-452, mir-483*, mir-331-3p, mir-743a, mir-485, mir-30c-1*, mir-770-5p, mir-483, mir-193, mir-296-5p, mir-715, or mir-712, Asth-miR-1 (SEQ NO:187), Asth-miR 2 (SEQ ID NO:189), or Asth-miR-5 (SEQ ID NO:195);
wherein an increase in the expression level of one or more of: mir-681, mir-880, mir-1190, mir-709, mir-671-3p, mir-1196, mir-667, mir-452, mir-483*, mir-331-3p, mir-743a, mir-485, mir-30c-1*, mir-770-5p, mir-483, mir-193, mir-296-5p, mir-715, mir-712, Asth-miR-1, or Asth-miR-2 in the lung cell indicates that the subject may therapeutically benefit from said inhibitor; and
wherein a decrease in the expression level of one or more of: mir-147, mir-135a, mir-135b, mir-683, mir-130b, mir-1, mir-615-5p, mir-142-3p, mir-689, mir-130b, mir-155, mir-146b, mir-18b, mir-340-5p, mir-501-5p, mir-1191, mir-421, mir-146b*, mir-717, mir-467c in the lung cell indicates that the subject may therapeutically benefit from said inhibitor.

68.-74. (canceled)
